SEQUENCE LISTING (40 sequences)

SEQ ID NO: 1 - DNA, 369 bp, synthetic construct
  misc_feature 1..369: note = Synthetic sequence
  source 1..369: mol_type = other DNA
gaggtccagctgcaacagtctggacctgaactggtgaagcctggggcttcagtaaagatgtcctgcaaggcttctagattcacattcactgactacaacatacactgggtgaagcagagccctggaaagacccttgaatggattggatatattaaccctaacaatggtgatattttctacaaacagaagttcaatggcaaggccacattgactataaacaagtcctccaacacagcctacatggagctccgcagcctgacatcggaggattctgcagtctattactgtgtaagatcctctaatattcgttatactttcgacaggttcttcgatgtctggggcacagggaccacggtcaccgtctcctca

SEQ ID NO: 2 - protein, 123 aa, synthetic construct
  REGION 1..123: note = Synthetic sequence
EVQLQQSGPELVKPGASVKMSCKASRFTFTDYNIHWVKQSPGKTLEWIGYINPNNGDIFYKQKFNGKATLTINKSSNTAYMELRSLTSEDSAVYYCVRSSNIRYTFDRFFDVWGTGTTVTVSS

SEQ ID NO: 3 - DNA, 318 bp, synthetic construct
  misc_feature 1..318: note = Synthetic sequence
  source 1..318: mol_type = other DNA
gaaaatgtgctcacccagtctccagcaatcatgtctgcatctctaggggagaaggtcaccatgagctgcagggccagctcaagtgtaaattacatttactggtaccagcagaagtcagatgcctcccccaaactatggatttattacacatccaacctggctcctggagtcccagctcgcttcagtggcagtgggtctgggaactcttattctctcacaatcagcagcatggagggtgaagatgctgccacttattactgccagcagttttctagttccccatccacgttcggtactgggaccaagctggagctgaaa

SEQ ID NO: 4 - protein, 106 aa, synthetic construct
  REGION 1..106: note = Synthetic sequence
ENVLTQSPAIMSASLGEKVTMSCRASSSVNYIYWYQQKSDASPKLWIYYTSNLAPGVPARFSGSGSGNSYSLTISSMEGEDAATYYCQQFSSSPSTFGTGTKLELK

SEQ ID NO: 5 - DNA, 732 bp, synthetic construct
  misc_feature 1..732: note = Synthetic sequence
  source 1..732: mol_type = other DNA
gaggtccagctgcaacagtctggacctgaactggtgaagcctggggcttcagtaaagatgtcctgcaaggcttctagattcacattcactgactacaacatacactgggtgaagcagagccctggaaagacccttgaatggattggatatattaaccctaacaatggtgatattttctacaaacagaagttcaatggcaaggccacattgactataaacaagtcctccaacacagcctacatggagctccgcagcctgacatcggaggattctgcagtctattactgtgtaagatcctctaatattcgttatactttcgacaggttcttcgatgtctggggcacagggaccacggtcaccgtctcctcaggcggaggcggatcaggtggtggcggatctggaggtggcggaagcgaaaatgtgctcacccagtctccagcaatcatgtctgcatctctaggggagaaggtcaccatgagctgcagggccagctcaagtgtaaattacatttactggtaccagcagaagtcagatgcctcccccaaactatggatttattacacatccaacctggctcctggagtcccagctcgcttcagtggcagtgggtctgggaactcttattctctcacaatcagcagcatggagggtgaagatgctgccacttattactgccagcagttttctagttccccatccacgttcggtactgggaccaagctggagctgaaa

SEQ ID NO: 6 - protein, 244 aa, synthetic construct
  REGION 1..244: note = Synthetic sequence
EVQLQQSGPELVKPGASVKMSCKASRFTFTDYNIHWVKQSPGKTLEWIGYINPNNGDIFYKQKFNGKATLTINKSSNTAYMELRSLTSEDSAVYYCVRSSNIRYTFDRFFDVWGTGTTVTVSSGGGGSGGGGSGGGGSENVLTQSPAIMSASLGEKVTMSCRASSSVNYIYWYQQKSDASPKLWIYYTSNLAPGVPARFSGSGSGNSYSLTISSMEGEDAATYYCQQFSSSPSTFGTGTKLELK

SEQ ID NO: 7 - DNA, 732 bp, synthetic construct
  misc_feature 1..732: note = Synthetic sequence
  source 1..732: mol_type = other DNA
caagtacagcttgtacaatccggagcggaagtaaagaagcccggggcctccgtgaaagtcagctgcaaagcgtctagattcaccttcactgactataacatccactgggtgcggcaagcgcctggtcagggcctcgaatggattggctatataaacccgaacaacggggacattttctataagcagaaattcaatggcagagtgacgctcaccgcggacaaaagtaccagtactgcttatatggaactgtctagtcttacgagtgaggataccgctgtgtattattgcgtgaggtcctccaatatacgctatacgtttgatagattctttgatgtatggggacagggaaccctcgtcacggtcagctcaggcggaggcggatcaggtggtggcggatctggaggtggcggaagcgacgtagtaatgactcaaagccccctctccttgccagtgacgccaggtgagccggcatctatttcttgcagggctagttcttcagtgaactacatctattggtatttgcagaagccaggacaaagtccgcagctttggatatattacacgagcaaccttgcgccaggtgttccagatcgcttctctggcagtggatcagggacggacttcacactgaagatctctcgcgttgaagctgaagacgtcggcgtgtactattgccaacaattcagtagttctccgagcacttttggccagggaaccaagcttgagattaaa

SEQ ID NO: 8 - protein, 244 aa, synthetic construct
  REGION 1..244: note = Synthetic sequence
QVQLVQSGAEVKKPGASVKVSCKASRFTFTDYNIHWVRQAPGQGLEWIGYINPNNGDIFYKQKFNGRVTLTADKSTSTAYMELSSLTSEDTAVYYCVRSSNIRYTFDRFFDVWGQGTLVTVSSGGGGSGGGGSGGGGSDVVMTQSPLSLPVTPGEPASISCRASSSVNYIYWYLQKPGQSPQLWIYYTSNLAPGVPDRFSGSGSGTDFTLKISRVEAEDVGVYYCQQFSSSPSTFGQGTKLEIK

SEQ ID NO: 9 - DNA, 732 bp, synthetic construct
  misc_feature 1..732: note = Synthetic sequence
  source 1..732: mol_type = other DNA
gacgtagtaatgactcaaagccccctctccttgccagtgacgccaggtgagccggcatctatttcttgcagggctagttcttcagtgaactacatctattggtatttgcagaagccaggacaaagtccgcagctttggatatattacacgagcaaccttgcgccaggtgttccagatcgcttctctggcagtggatcagggacggacttcacactgaagatctctcgcgttgaagctgaagacgtcggcgtgtactattgccaacaattcagtagttctccgagcacttttggccagggaaccaagcttgagattaaaggcggaggcggatcaggtggtggcggatctggaggtggcggaagccaagtacagcttgtacaatccggagcggaagtaaagaagcccggggcctccgtgaaagtcagctgcaaagcgtctagattcaccttcactgactataacatccactgggtgcggcaagcgcctggtcagggcctcgaatggattggctatataaacccgaacaacggggacattttctataagcagaaattcaatggcagagtgacgctcaccgcggacaaaagtaccagtactgcttatatggaactgtctagtcttacgagtgaggataccgctgtgtattattgcgtgaggtcctccaatatacgctatacgtttgatagattctttgatgtatggggacagggaaccctcgtcacggtcagctca

SEQ ID NO: 10 - protein, 244 aa, synthetic construct
  REGION 1..244: note = Synthetic sequence
DVVMTQSPLSLPVTPGEPASISCRASSSVNYIYWYLQKPGQSPQLWIYYTSNLAPGVPDRFSGSGSGTDFTLKISRVEAEDVGVYYCQQFSSSPSTFGQGTKLEIKGGGGSGGGGSGGGGSQVQLVQSGAEVKKPGASVKVSCKASRFTFTDYNIHWVRQAPGQGLEWIGYINPNNGDIFYKQKFNGRVTLTADKSTSTAYMELSSLTSEDTAVYYCVRSSNIRYTFDRFFDVWGQGTLVTVSS

SEQ ID NO: 11 - DNA, 729 bp, synthetic construct
  misc_feature 1..729: note = Synthetic sequence
  source 1..729: mol_type = other DNA
caggtccagcttgtccagtctggtgctgaggttaaaaaacctggcgcaagcgtgaaggtgagctgtaaggctagccgatttacctttaccgactacaacatacattgggtcaggcaagcgcctggacaaaggctcgagtggattggttacataaacccaaataacggggacatattttacaaacagaaattcaacggccgggtaactataacacgagacacaagtgcaagtaccgcctatatggagctctcttcccttcgatccgaagacacggcagtctactactgcgttcgcagctccaatatccgctatacctttgataggttctttgatgtgtggggtcaagggacgctcgtaaccgtgagcggaggaggcggttctggtggcgggggcagcggtgggggagggtctgatgtcgttatgactcagagtccagcgtttctcagtgttacacccggtgaaaaggtcaccattacgtgccgggcgagctcttcagttaactacatttattggtaccagcagaagccagatcaggcgcccaaactttggatttactataccagcaaccttgcacccggtgtacccagtcggtttagcggcagtgggagtggtactgacttcacttttactatttcatcattggaggctgaagatgctgcgacatactattgtcagcaattcagctcctctcccagcacctttggccagggcaccaaacttgaaattaaa

SEQ ID NO: 12 - protein, 243 aa, synthetic construct
  REGION 1..243: note = Synthetic sequence
QVQLVQSGAEVKKPGASVKVSCKASRFTFTDYNIHWVRQAPGQRLEWIGYINPNNGDIFYKQKFNGRVTITRDTSASTAYMELSSLRSEDTAVYYCVRSSNIRYTFDRFFDVWGQGTLVTVSGGGGSGGGGSGGGGSDVVMTQSPAFLSVTPGEKVTITCRASSSVNYIYWYQQKPDQAPKLWIYYTSNLAPGVPSRFSGSGSGTDFTFTISSLEAEDAATYYCQQFSSSPSTFGQGTKLEIK

SEQ ID NO: 13 - DNA, 729 bp, synthetic construct
  misc_feature 1..729: note = Synthetic sequence
  source 1..729: mol_type = other DNA
gatgtcgttatgactcagagtccagcgtttctcagtgttacacccggtgaaaaggtcaccattacgtgccgggcgagctcttcagttaactacatttattggtaccagcagaagccagatcaggcgcccaaactttggatttactataccagcaaccttgcacccggtgtacccagtcggtttagcggcagtgggagtggtactgacttcacttttactatttcatcattggaggctgaagatgctgcgacatactattgtcagcaattcagctcctctcccagcacctttggccagggcaccaaacttgaaattaaaggaggaggcggttctggtggcgggggcagcggtgggggagggtctcaggtccagcttgtccagtctggtgctgaggttaaaaaacctggcgcaagcgtgaaggtgagctgtaaggctagccgatttacctttaccgactacaacatacattgggtcaggcaagcgcctggacaaaggctcgagtggattggttacataaacccaaataacggggacatattttacaaacagaaattcaacggccgggtaactataacacgagacacaagtgcaagtaccgcctatatggagctctcttcccttcgatccgaagacacggcagtctactactgcgttcgcagctccaatatccgctatacctttgataggttctttgatgtgtggggtcaagggacgctcgtaaccgtgagc

SEQ ID NO: 14 - protein, 243 aa, synthetic construct
  REGION 1..243: note = Synthetic sequence
DVVMTQSPAFLSVTPGEKVTITCRASSSVNYIYWYQQKPDQAPKLWIYYTSNLAPGVPSRFSGSGSGTDFTFTISSLEAEDAATYYCQQFSSSPSTFGQGTKLEIKGGGGSGGGGSGGGGSQVQLVQSGAEVKKPGASVKVSCKASRFTFTDYNIHWVRQAPGQRLEWIGYINPNNGDIFYKQKFNGRVTITRDTSASTAYMELSSLRSEDTAVYYCVRSSNIRYTFDRFFDVWGQGTLVTVS

SEQ ID NO: 15 - DNA, 732 bp, synthetic construct
  misc_feature 1..732: note = Synthetic sequence
  source 1..732: mol_type = other DNA
caagtgcagttggtgcaatcaggagcggaagtcaagaaaccgggggcatctgtcaaagtgagctgcaaggcgagccggttcacatttacggattacaacatacattgggtgcgccaagccccgggacagggcctcgaatggataggctacatcaatcctaataatggggatatcttctataagcagaaatttaatggaaaggcaaccatgacagtagatacttctactagcacagtttacatggagctgtcctcactgcggtctgaggatacggcggtgtactattgcgttaggagcagcaatatacggtacacgttcgatcgcttcttcgatgtttggggccagggcaccctcgtgacggtatctggaggaggcgggtcaggtggcggtggctcaggcgggggggggagtatggacatccagatgacccagagccctagcagtttgtcagcttccgtgggagacagagtcaccataacatgtcgagcatccagtagtgtcaactatatatactggtatcaacagaagtccggcaaggcgcctaaactgtggatttattatacgtccaacctcgcacccggcgttccaagccgattctccggcagtggatcaggaaccgacttcacgctgacgatcagtagtcttcagccagaggatttcgctacatattactgtcaacagttttcctcaagccctagtacctttgggcagggaaccaaactggagataaag

SEQ ID NO: 16 - protein, 244 aa, synthetic construct
  REGION 1..244: note = Synthetic sequence
QVQLVQSGAEVKKPGASVKVSCKASRFTFTDYNIHWVRQAPGQGLEWIGYINPNNGDIFYKQKFNGKATMTVDTSTSTVYMELSSLRSEDTAVYYCVRSSNIRYTFDRFFDVWGQGTLVTVSGGGGSGGGGSGGGGSMDIQMTQSPSSLSASVGDRVTITCRASSSVNYIYWYQQKSGKAPKLWIYYTSNLAPGVPSRFSGSGSGTDFTLTISSLQPEDFATYYCQQFSSSPSTFGQGTKLEIK

SEQ ID NO: 17 - DNA, 732 bp, synthetic construct
  misc_feature 1..732: note = Synthetic sequence
  source 1..732: mol_type = other DNA
atggacatccagatgacccagagccctagcagtttgtcagcttccgtgggagacagagtcaccataacatgtcgagcatccagtagtgtcaactatatatactggtatcaacagaagtccggcaaggcgcctaaactgtggatttattatacgtccaacctcgcacccggcgttccaagccgattctccggcagtggatcaggaaccgacttcacgctgacgatcagtagtcttcagccagaggatttcgctacatattactgtcaacagttttcctcaagccctagtacctttgggcagggaaccaaactggagataaagggaggaggcgggtcaggtggcggtggctcaggcgggggggggagtcaagtgcagttggtgcaatcaggagcggaagtcaagaaaccgggggcatctgtcaaagtgagctgcaaggcgagccggttcacatttacggattacaacatacattgggtgcgccaagccccgggacagggcctcgaatggataggctacatcaatcctaataatggggatatcttctataagcagaaatttaatggaaaggcaaccatgacagtagatacttctactagcacagtttacatggagctgtcctcactgcggtctgaggatacggcggtgtactattgcgttaggagcagcaatatacggtacacgttcgatcgcttcttcgatgtttggggccagggcaccctcgtgacggtatct

SEQ ID NO: 18 - protein, 244 aa, synthetic construct
  REGION 1..244: note = Synthetic sequence
MDIQMTQSPSSLSASVGDRVTITCRASSSVNYIYWYQQKSGKAPKLWIYYTSNLAPGVPSRFSGSGSGTDFTLTISSLQPEDFATYYCQQFSSSPSTFGQGTKLEIKGGGGSGGGGSGGGGSQVQLVQSGAEVKKPGASVKVSCKASRFTFTDYNIHWVRQAPGQGLEWIGYINPNNGDIFYKQKFNGKATMTVDTSTSTVYMELSSLRSEDTAVYYCVRSSNIRYTFDRFFDVWGQGTLVTVS

SEQ ID NO: 19 - DNA, 729 bp, synthetic construct
  misc_feature 1..729: note = Synthetic sequence
  source 1..729: mol_type = other DNA
caggtgcagctggtccagagtggggcagaagtcaagaaacccggagcaagtgtcaaagtgtcctgtaaagcctcacgattcacatttaccgactataacatccactgggtgagacaggcaccaggacagaggctggagtggatcggctatatcaaccctaacaatggcgacatcttctacaagcagaagtttaatggccgggtgaccatcacaagagataccagcgcctccacagcctatatggagctgagctccctgcggagcgaggataccgccgtgtactattgcgtgaggtctagcaatatccgctacacattcgaccggttctttgacgtgtggggccagggcaccctggtgacagtgtccggcggcggcggctctggcggaggaggcagcggcggaggaggctccgagatcgtgctgacccagtctcctgccacactgtctctgagcccaggagagagggccaccctgtcctgtagggcctcctctagcgtgaactacatctattggtaccagcagaagccaggccaggcccccagactgtggatctactatacctccaatctggcaccaggcatccctgcaaggttctccggctctggcagcggcacagactttaccctgacaatctcctctctggagcccgaggattttgccgtgtactattgtcagcagtttagcagttcaccaagcacattcgggcagggcaccaagctggaaatcaag

SEQ ID NO: 20 - protein, 243 aa, synthetic construct
  REGION 1..243: note = Synthetic sequence
QVQLVQSGAEVKKPGASVKVSCKASRFTFTDYNIHWVRQAPGQRLEWIGYINPNNGDIFYKQKFNGRVTITRDTSASTAYMELSSLRSEDTAVYYCVRSSNIRYTFDRFFDVWGQGTLVTVSGGGGSGGGGSGGGGSEIVLTQSPATLSLSPGERATLSCRASSSVNYIYWYQQKPGQAPRLWIYYTSNLAPGIPARFSGSGSGTDFTLTISSLEPEDFAVYYCQQFSSSPSTFGQGTKLEIK

SEQ ID NO: 21 - DNA, 729 bp, synthetic construct
  misc_feature 1..729: note = Synthetic sequence
  source 1..729: mol_type = other DNA
gagatcgtgctgacccagtctcctgccacactgtctctgagcccaggagagagggccaccctgtcctgtagggcctcctctagcgtgaactacatctattggtaccagcagaagccaggccaggcccccagactgtggatctactatacctccaatctggcaccaggcatccctgcaaggttctccggctctggcagcggcacagactttaccctgacaatctcctctctggagcccgaggattttgccgtgtactattgtcagcagtttagcagttcaccaagcacattcgggcagggcaccaagctggaaatcaagggcggcggcggctctggcggaggaggcagcggcggaggaggctcccaggtgcagctggtccagagtggggcagaagtcaagaaacccggagcaagtgtcaaagtgtcctgtaaagcctcacgattcacatttaccgactataacatccactgggtgagacaggcaccaggacagaggctggagtggatcggctatatcaaccctaacaatggcgacatcttctacaagcagaagtttaatggccgggtgaccatcacaagagataccagcgcctccacagcctatatggagctgagctccctgcggagcgaggataccgccgtgtactattgcgtgaggtctagcaatatccgctacacattcgaccggttctttgacgtgtggggccagggcaccctggtgacagtgtcc

SEQ ID NO: 22 - protein, 243 aa, synthetic construct
  REGION 1..243: note = Synthetic sequence
EIVLTQSPATLSLSPGERATLSCRASSSVNYIYWYQQKPGQAPRLWIYYTSNLAPGIPARFSGSGSGTDFTLTISSLEPEDFAVYYCQQFSSSPSTFGQGTKLEIKGGGGSGGGGSGGGGSQVQLVQSGAEVKKPGASVKVSCKASRFTFTDYNIHWVRQAPGQRLEWIGYINPNNGDIFYKQKFNGRVTITRDTSASTAYMELSSLRSEDTAVYYCVRSSNIRYTFDRFFDVWGQGTLVTVS

SEQ ID NO: 23 - DNA, 117 bp, Homo sapiens
  source 1..117: mol_type = genomic DNA
atcgaagtcatgtatccccccccctatctggacaacgaaaagagtaacggaactatcattcacgtcaaaggaaaacacctgtgccctagcccactgttccccggcccttccaagccc

SEQ ID NO: 24 - protein, 39 aa, Homo sapiens
IEVMYPPPYLDNEKSNGTIIHVKGKHLCPSPLFPGPSKP

SEQ ID NO: 25 - DNA, 81 bp, Homo sapiens
  source 1..81: mol_type = genomic DNA
ttttgggtgctggtggtggtgggcggcgtgctggcttgctattccctgctggtcacagtcgcttttattattttctgggtg

SEQ ID NO: 26 - protein, 27 aa, Homo sapiens
FWVLVVVGGVLACYSLLVTVAFIIFWV

SEQ ID NO: 27 - DNA, 126 bp, Homo sapiens
  source 1..126: mol_type = genomic DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 28 - protein, 42 aa, Homo sapiens
KRGRKKLLYIFKQPFMRPVQTTQEEDGCSCRFPEEEEGGCEL

SEQ ID NO: 29 - DNA, 336 bp, Homo sapiens
  source 1..336: mol_type = genomic DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 30 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - DNA, 66 bp, Homo sapiens
  source 1..66: mol_type = genomic DNA
atgcttctcctggtgacaagccttctgctctgtgagttaccacacccagcattcctcctgatccca

SEQ ID NO: 32 - protein, 22 aa, Homo sapiens
MLLLVTSLLLCELPHPAFLLIP

SEQ ID NO: 33 - DNA, 54 bp, synthetic construct
  misc_feature 1..54: note = Synthetic sequence
  source 1..54: mol_type = other DNA
gagggcagaggaagtcttctaacatgcggtgacgtggaggagaatcccggccct

SEQ ID NO: 34 - protein, 18 aa, synthetic construct
  REGION 1..18: note = Synthetic sequence
EGRGSLLTCGDVEENPGP

SEQ ID NO: 35 - DNA, 1005 bp, Homo sapiens
  source 1..1005: mol_type = genomic DNA
cgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatg

SEQ ID NO: 36 - protein, 335 aa, Homo sapiens
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 37 - DNA, 2598 bp, synthetic construct
  misc_feature 1..2598: note = Synthetic sequence
  source 1..2598: mol_type = other DNA
atgcttctcctggtgacaagccttctgctctgtgagttaccacacccagcattcctcctgatcccacatatggaggtccagctgcaacagtctggacctgaactggtgaagcctggggcttcagtaaagatgtcctgcaaggcttctagattcacattcactgactacaacatacactgggtgaagcagagccctggaaagacccttgaatggattggatatattaaccctaacaatggtgatattttctacaaacagaagttcaatggcaaggccacattgactataaacaagtcctccaacacagcctacatggagctccgcagcctgacatcggaggattctgcagtctattactgtgtaagatcctctaatattcgttatactttcgacaggttcttcgatgtctggggcacagggaccacggtcaccgtctcctcaggcggaggcggatcaggtggtggcggatctggaggtggcggaagcgaaaatgtgctcacccagtctccagcaatcatgtctgcatctctaggggagaaggtcaccatgagctgcagggccagctcaagtgtaaattacatttactggtaccagcagaagtcagatgcctcccccaaactatggatttattacacatccaacctggctcctggagtcccagctcgcttcagtggcagtgggtctgggaactcttattctctcacaatcagcagcatggagggtgaagatgctgccacttattactgccagcagttttctagttccccatccacgttcggtactgggaccaagctggagctgaaaactagtatcgaagtcatgtatccccccccctatctggacaacgaaaagagtaacggaactatcattcacgtcaaaggaaaacacctgtgccctagcccactgttccccggcccttccaagcccttttgggtgctggtggtggtgggcggcgtgctggcttgctattccctgctggtcacagtcgcttttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgagggcagaggaagtcttctaacatgcggtgacgtggaggagaatcccggccctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 38 - protein, 865 aa, synthetic construct
  REGION 1..865: note = Synthetic sequence
MLLLVTSLLLCELPHPAFLLIPHMEVQLQQSGPELVKPGASVKMSCKASRFTFTDYNIHWVKQSPGKTLEWIGYINPNNGDIFYKQKFNGKATLTINKSSNTAYMELRSLTSEDSAVYYCVRSSNIRYTFDRFFDVWGTGTTVTVSSGGGGSGGGGSGGGGSENVLTQSPAIMSASLGEKVTMSCRASSSVNYIYWYQQKSDASPKLWIYYTSNLAPGVPARFSGSGSGNSYSLTISSMEGEDAATYYCQQFSSSPSTFGTGTKLELKTSIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREGRGSLLTCGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 39 - protein, 865 aa, synthetic construct
  REGION 1..865: note = Synthetic sequence
MLLLVTSLLLCELPHPAFLLIPHMEVQLQQSGPELVKPGASVKMSCKASRFTFTDYNIHWVKQSPGKTLEWIGYINPNNGDIFYKQKFNGKATLTINKSSNTAYMELRSLTSEDSAVYYCVRSSNIRYTFDRFFDVWGTGTTVTVSSGGGGSGGGGSGGGGSENVLTQSPAIMSASLGEKVTMSCRASSSVNYIYWYQQKSDASPKLWIYYTSNLAPGVPARFSGSGSGNSYSLTISSMEGEDAATYYCQQFSSSPSTFGTGTKLELKTSTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREGRGSLLTCGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 40 - protein, 912 aa, synthetic construct
  REGION 1..912: note = Synthetic sequence
MLLLVTSLLLCELPHPAFLLIPHMEVQLQQSGPELVKPGASVKMSCKASRFTFTDYNIHWVKQSPGKTLEWIGYINPNNGDIFYKQKFNGKATLTINKSSNTAYMELRSLTSEDSAVYYCVRSSNIRYTFDRFFDVWGTGTTVTVSSGGGGSGGGGSGGGGSENVLTQSPAIMSASLGEKVTMSCRASSSVNYIYWYQQKSDASPKLWIYYTSNLAPGVPARFSGSGSGNSYSLTISSMEGEDAATYYCQQFSSSPSTFGTGTKLELKTSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREGRGSLLTCGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

(19) United States
(12) Patent Application Publication
(10) Pub. No.: US 2025/0144214 A1
(43) Pub. Date: May 8, 2025

(54) CD28 HINGE AND TRANSMEMBRANE CONTAINING CHIMERIC ANTIGEN RECEPTORS TARGETING GPC2 AND USE THEREOF

(71) Applicant: The U.S.A., as represented by the Secretary, Department of Health and Human Services, Betlhesda, MD (US)

(72) Inventors: Mitchell Ho, Urbana, MD (US); Carol J. Thiele, Bethesda, MD (US); Nan Li, Laurel, MD (US); Rosa Nguyen, Washington, DC (US); Rosandra N. Kaplan, Potomac, MD (US)

(73) Assignee: The U.S.A., as represented by the Secretary, Department of Health and Human Services, Bethesda, MD (US)

(21) Appl. No.: 18/837,367

(22) PCT Filed: Feb. 14, 2023
(86) PCT No.: PCT/US2023/062525

§ 371 Date: Aug. 9, 2024

Related U.S. Application Data
(60) Provisional application No. 63/310,456, filed on Feb. 15, 2022.

(51) Int. Cl. A61K 40/42; A61K 40/11; A61K 40/31; A61P 35/00; C07K 14/725; C07K 16/30

(52) U.S. Cl. CPC A61K 40/4261; A61K 40/11; A61K 40/31; A61P 35/00; C07K 14/7051; C07K 16/30; C07K 2317/24; C07K 2317/92

(57) ABSTRACT

Optimized chimeric antigen receptors (CARs) targeting glypican-2 (GPC2) and having a CD28 hinge region and a CD28 transmembrane domain are described. The antigen-binding domain of the disclosed CARs is derived from GPC2-specific antibody CT3 or humanized versions thereof. The optimized CARs also include an intracellular co-stimulatory domain and an intracellular signaling domain. Immune cells or induced pluripotent stem cells expressing the optimized CARs can be used to treat GPC2-positive solid tumors, such as neuroblastoma.

### CROSS REFERENCE TO RELATED APPLICATIONS

This application claims the benefit of U.S. Provisional Application No. 63/310,456, filed Feb. 15, 2022, which is herein incorporated by reference in its entirety.

### STATEMENT OF GOVERNMENT SUPPORT

This invention was made with government support under project numbers ZO1 BC010891, ZIA BC010891, ZO1 ZIA BC010788, ZIA BC011334, and ZIA BC 012066 awarded by the National Institutes of Health, National Cancer Institute. The government has certain rights in the invention.

### FIELD OF THE DISCLOSURE

This disclosure concerns optimized chimeric antigen receptors (CARs) specific for tumor antigen glypican-2 (GPC2) that include a hinge region and transmembrane domain derived from CD28. This disclosure further concerns use of the GPC2-targeted CARs, such as for treating solid tumors.

### INCORPORATION OF ELECTRONIC SEQUENCE LISTING

The electronic sequence listing, submitted herewith as an XML file named 4239-107434-02.xml (63,252 bytes), created on Jan. 26, 2023, is herein incorporated by reference in its entirety.

### BACKGROUND

CAR T cell therapies have emerged as an important class of cancer therapeutics and are being actively developed and tested worldwide. Following the initial success in hematological cancers using CD19-targeted CAR T cells, various CAR strategies have been rigorously engineered and tested with the goal of treating solid tumors.

Glypican-2 (GPC2) is one member of the six-member glypican family of heparan sulfate proteoglycans that are attached to the cell surface by a glycosylphosphatidylinositol (GPI) anchor (Li et al., Trends Cancer 4(11):741-754, 2018). GPC2 mRNA and protein are elevated in neuroblastoma and other pediatric cancers (Orentas et al., Front Oncol 2:194, 2012; Li et al., Proc Natl Acad Sci USA 114(32):E6623-E6631, 2017; WO 2020/033430; and WO 2018/026533).

Neuroblastoma is the most common type of extracranial solid tumor in children. Derived from neuroendocrine tissue of the sympathetic nervous system, it accounts for about 8-10% of childhood cancers in the United States (Maris and Hogarty, Lancet 369:2106-2120, 2007). Neuroblastoma is a complex and heterogeneous disease, with nearly 50% of patients having a high-risk phenotype characterized by widespread dissemination of the cancer and poor long-term survival even if intensive multimodal treatments are used (Yu et al., New Engl J Med 363:1324-1334, 2010). Approximately 45% of patients receiving standard therapy have a relapse and ultimately die from metastatic disease (Matthay et al., New Engl J Med 341:1165-1173, 1999). As such, there is an urgent and unmet need for a safe and effective treatment of neuroblastoma.

### SUMMARY

Disclosed herein are optimized GPC2-specific chimeric antigen receptors (CARs) that include a hinge region and a transmembrane domain derived from human CD28. It is demonstrated herein that GPC2-specific CARs having a CD28 hinge and CD28 transmembrane domain are surprisingly more effective at killing GPC2-positive cells in vitro and eradicating GPC2-positive tumors in animal models relative to GPC2-specific CARs having a hinge region derived from CD8 and a transmembrane (TM) domain derived from either CD8 or CD28.

Provided herein are CARs that include an extracellular antigen-binding domain specific for GPC2; a CD28 hinge region; a CD28 transmembrane domain; an intracellular co-stimulatory domain; and an intracellular signaling domain. In some aspects, the antigen-binding domain includes a variable heavy (VH) domain and a variable light (VL) domain, and the VH and VL domains include the CDR sequences of GPC2-specific antibody CT3 or a humanized version thereof (such as hCT3-1, hCT3-2, hCT3-3 or hCT3-4). In some examples, the antigen-binding domain includes a linker sequence between the VH domain and the VL domain, and the antigen-binding domain can be in a VH-linker-VL orientation or a VL-linker-VH orientation.

Nucleic acid molecules encoding a disclosed CAR are further provided. In some aspects, the nucleic acid molecule includes in the 5′ to 3′ direction a nucleic acid encoding a first granulocyte-macrophage colony stimulating factor receptor signal sequence (GMCSFRss); a nucleic acid encoding the antigen-binding domain; a nucleic acid encoding the CD28 hinge region; a nucleic acid encoding the CD28 transmembrane domain; a nucleic acid encoding the co-stimulatory domain; a nucleic acid encoding the signaling domain; a nucleic acid encoding a self-cleaving 2A peptide; a nucleic acid encoding a second GMCSFRss; and a nucleic acid encoding a truncated human epidermal growth factor receptor (hEGFRt). In some examples, the nucleic acid molecule further includes a human elongation factor 1α (EF1α) promoter sequence 5′ of the nucleic acid encoding the first GMCSFRss. Vectors (such as lentiviral vectors) that include the disclosed nucleic acid molecules are further provided.

Also provided are isolated immune cells (such as T cells, NK cells, B cells or macrophages) and induced pluripotent stem cells (iPSCs) expressing a CAR disclosed herein and/or containing an isolated nucleic acid molecule or vector encoding a CAR disclosed herein.

Further provided are compositions that include a pharmaceutically acceptable carrier and a CAR, nucleic acid molecule, vector or cell disclosed herein.

Methods of treating a GPC2-positive cancer or inhibiting tumor growth or metastasis of a GPC2-positive cancer in a subject are also provided. In some aspects, the methods include administering to the subject a therapeutically effective amount of a CAR, nucleic acid molecule, vector, cell or composition disclosed herein. In some examples, the GPC2-positive cancer is a solid tumor, such as neuroblastoma, medulloblastoma, or retinoblastoma.

The foregoing and other features of this disclosure will become more apparent from the following detailed description of several aspects which proceeds with reference to the accompanying figures.

### BRIEF DESCRIPTION OF THE DRAWINGS

FIGS. 1A-1B: CAR constructs. (FIG. 1A) Schematic of CAR constructs CT3.8H.BBz, CT3.8H.28BBz and CT3.28H.BBz. (FIG. 1B) Detailed schematic of the CT3.28H.BBz CAR design.

FIGS. 2A-2B: In vitro cell killing assay of GPC2-expressing IMR5 cells (FIG. 2A) and GPC2 knockout (KO) IMR5 cells (FIG. 2B). CT3.28H.BBz CAR T cells were more potent than CT3.8H.BBz CAR T cells at killing IMR5 cells. Both types of CAR T cells had minimal effect on GPC2-KO IMR5 cells, demonstrating their GPC2 specificity.

FIGS. 3A-3D: Comparison of CT3.8H.BBz and CT3.28H.BBz CAR T cells in an IMR5 metastasis model. (FIG. 3A) Experimental design. Mice were i.v. inoculated with IMR5-luc 28 days prior to infusion with 10 million CAR T cells. Mice were imaged weekly after infusion. (FIG. 3B) Bioluminescence images of mock and CAR T cell-treated mice. (FIG. 3C) Bioluminescence measured two, four, six and eight weeks after CAR T cell infusion. (FIG. 3D) Survival of mock and CAR T cell-treated mice following CAR T cell infusion. CT3.28H.BBz CAR T cells were significantly more potent than CT3.8H.BBz CAR T cells in regressing neuroblastoma tumors in mice. All mice in the CT3.28H.BBz treatment group survived to the end of the study.

FIGS. 4A-4G: Comparison of CT3.28H.BBz and CT3.8H.28BBz CAR T cells in an orthotopic IMR5 mouse model. (FIGS. 4A-4C) T cells from three different human donors, A26M (FIG. 4A), A59F (FIG. 4B) and A25F (FIG. 4C), were used. Mice with moderate tumor burden were administered 5 million human T cells expressing CT3.28H.BBz CAR or CT3.8H.28BBz CAR. CT3.28H.BBz CAR T cells were superior to CT3.8H.28BBz CAR T cells for all three donors. (FIGS. 4D-4E) Bioluminescence images of mock-treated mice and mice treated with CT3.28H.BBz or CT3.8H.28BBz CAR T cells derived from donor A26M (FIG. 4D) and donor A59F (FIG. 4E). CT3.28H.BBz CAR T cells were more potent than CT3.8H.28BBz CAR T cells in eradicating moderately sized IMR5 tumors. (FIG. 4F) Flow cytometry plots showing gating strategy of flow samples. Live cells were gated for CD3+ human cells and the percentage of CAR-positive cells was determined. (FIG. 4G) Graph showing the percentage of CD3+ cells expressing CT3.28H.BBz CAR or CT3.8H.28BBz CAR for donors A26M and A59F. For both donors, CT3.28H.BBz CAR T cells retained higher levels of CAR expression than CT3.8H.28BBz CAR T cells.

FIGS. 5A-5B: Detection of CAR phosphorylation as a measure of CAR activation. (FIG. 5A) CT3.8H.BBz, CT3.8H.28BBz and CT3.28H.BBz CAR T cells were unstimulated or stimulated with Protein L or GPC2-Fc and CAR phosphorylation was detected by Western blot. (FIG. 5B) Graph showing fold change in CAR phosphorylation. CT3.8H.28BBz CAR had higher phosphorylation levels than CT3.28H.BBz CAR when tested without stimulation. With GPC2 stimulation, both CARs upregulated their phosphorylation levels. These results demonstrate that CT3.8H.28BBz has more tonic CAR signaling (phosphorylation), which is known to lead to CAR exhaustion. CT3.28H.BBz CAR T cells have lower tonic CAR signaling but show appropriate CAR activation upon antigen presentation (GPC2-Fc).

FIGS. 6A-6B: Comparison of CT3.28H.BBz CAR T cells and CT3.8H.28BBz CAR T cells with low-dose or high-dose chemotherapy in an orthotopic IMR5 animal model. Mice with a large tumor burden were treated with no chemotherapy, low-dose chemotherapy, or high-dose chemotherapy (fludarabine/cyclophosphamide) for three days prior to infusion of 5 million CAR T cells. (FIG. 6A) Tumor size as measured by bioluminescence. (FIG. 6B) Tumor weight 10 weeks after chemotherapy and CAR T cell infusion. CT3.28H.BBz outperformed CT3.8H.28BBz when given with low-dose conditioning chemotherapy in high-tumor burden mice.

FIGS. 7A-7B: Binding affinity of humanized CT3 (hCT3) antibodies. Shown is a set of graphs demonstrating that binding affinity of the four humanized CT3 antibodies (4.0 nM, 3.6 nM, 2.5 nM and 3.3 nM) is similar to the binding affinity of the parental CT3 antibody (2.2 nM).

FIG. 8: Humanized CT3 antibodies bind cell-surface GPC2. Shown are flow cytometry plots demonstrating that all four humanized CT3 antibodies maintain the ability to bind cell-surface GPC2.

FIG. 9: Cell killing by humanized CT3-based CAR T cells. The graph shows specific lysis of GPC2-expressing IMR5 cells by CT3-8H-BBz, hCT3-1-8H-BBz, hCT3-2-8H-BBz, hCT3-3-8H-BBz and hCT3-4-8H-BBz CAR T cells. All four humanized CT3-8H-BBz CAR T cells showed improved killing activity against IMR5 cells as compared to CT3-8H-BBz CAR T cells.

FIG. 10: Schematic of two humanized CT3-28H-BBz CAR constructs in which the GPC2-binding domain has a VH-linker-VL orientation (top) or a VL-linker-VH orientation (bottom).

FIGS. 11A-11D: Comparison of three GPC2-CAR constructs in vitro. (FIG. 11A) Schema of the three CAR constructs used for preclinical studies with variable hinge, TM, and co-stimulatory domains. (FIG. 111B) Cytokine secretion profile of GPC2-CAR T cells at 24 hours in co-culture with tumor cells. Human T cells transduced with the three different GPC2-CARs were grown with GPC2-KO or GPC2-WT IMR-5 tumor cells. The levels of interferon-γ (IFNγ), granzyme B (GZMB), and soluble Fas ligand (sFASL) were significantly increased in the presence of GPC2 but comparable across the three tested CARs. (FIG. 11C) Western blot analysis to demonstrate CAR signaling in resting and CAR-crosslinked T cells. (FIG. 11D) Densiometric quantification of western blot signals from FIG. 11C. CT3.8H.28BBζ shows prominent tonic signaling of the CAR at rest, which is lacking for CT3.28H.BBζ and CT3.8H.BBζ. However, antigen-specific CAR activation induces higher phosphorylation levels in CT3.28H.BBζ compared with CT3.8H.BBζ.

FIGS. 12A-12E: CT3.28H.BBζ outperforms CT3.8H.CD28BBζ in vivo. (FIG. 12A) Tumor weights on Day 50 post tumor injection. CT3.28H.BBζ induced the most significant tumor regression across all therapy groups. (FIG. 12B) Model system and experimental regimen. (FIG. 12C) Longitudinal bioluminescence imaging (BLI) signals of mice treated with untransduced (UT) mock T cells or untreated controls and GPC2-targeted CAR T cells. Weekly BLI revealed that CT3.28H.BBζ-CAR T-treated animals demonstrated a profound decline in their BLI signal that was sustained for the duration of the study. In contrast, mice treated with CT3.8H.28BBζ or lower cell doses of each of the GPC2-targeted CAR T cells showed a temporary response but ultimately progressed. (FIG. 12D) Survival curves of mice from FIG. 12C. (FIG. 12E) Persistence of CAR⁺ T cells isolated from the tumor of treated mice in FIG. 12C on Day 80 by flow cytometry analysis.

FIGS. 13A-13K: GPC2-CAR T cell manufacturing yields proliferating and cytotoxic effector T cells. (FIG. 13A) Proportions of captured immune cells on Day 8 of CAR T manufacturing. (FIG. 13B) Uniform manifold approximation (UMAP), (FIG. 13C) CD8 and CD4 protein expression levels detected by TotalSeq, (FIG. 13D) cluster annotations, and (FIG. 13E) fractions of subsets of manufactured cells from Donor 1. (FIG. 13F) UMAP, (FIG. 13G) CD8 and CD4 protein expression levels, (FIG. 13H) cluster annotations, and (FIG. 13I) fractions of subsets of manufactured cells from Donor 2. (FIGS. 13J-13K) Differentially expressed genes (DEG) analysis of the produced cells.

FIGS. 14A-14J: GPC2-CAR T cells home to the tumor microenvironment (TME) and enrich as a cytotoxic effector population in vivo. (FIG. 14A) GPC2-targeted CAR T cell tracking using BLI in vivo. T cells were transduced to express firefly luciferase (ffLUC)-GFP and were monitored serially for homing and expansion. (FIGS. 14B-14C) All three CARs enrich and expand in the TME compared with UT mock cells. *p<0.05; Student's t-test. (FIGS. 14D-14E) IMR-5-bearing mice underwent T cell injection. Eight days later, the tumors were isolated and single-cell RNA-seq was performed. Quantifications of tumor and immune cells derived from the tumor are shown. (FIGS. 14F-14G) UMAP plots showing tumor cells and five immune subsets. The bar graphs quantify the immune proportions. (FIG. 14H) Volcano plots of differentially expressed genes comparing CT3.28H.BBζ vs CT3.8H.BBζ (top panels) and CT3.28H.BBζ vs CT3.8H.28BBζ (bottom panels). (FIG. 14I) Differentially expressed genes grouped by function and extracted from both comparisons made in FIG. 14H. (FIG. 14J) Compound pathway analysis reveals that CT3.28H.BBζ CAR T cells upregulate the granzyme A pathway but downregulate pathways related to mitochondrial oxidative phosphorylation (OXPHOS) and eukaryotic initiation factor 2 (EIF2). Abbreviations: CTLA-4, cytotoxic T-lymphocytes-associated protein 4; mTOR, mammalian target of rapamycin; TAM, tumor-associated macrophage; pDC, plasmacytoid dendritic cells; PD-1; programmed cell death protein-1; PD-L1, programmed death-ligand 1; seq, sequencing.

FIGS. 15A-15F: Head-to-head comparison of the GPC2-CAR (CT3.28H.BBζ) versus a GD2-CAR (K666.28H.BBζ). (FIG. 15A) Transduction efficiencies of CT3.28H.BBζ and K666.28H.BBζ CAR T cells. (FIG. 15B) In vitro cytotoxicity assays testing both CARs against three NB lines at varying E:T ratios. ***p<0.001; ****p<0.0001; two-way analysis of variance. (FIG. 15C) In vitro tumor rechallenge assay. CAR T cells were rechallenged every 24 hours. The cytotoxic activity was measured at 24 hours, 96 hours, and 7 days. *p<0.05; paired t-test. (FIG. 15D) Tumor weights on Day 50 post tumor implantation. *p<0.05; Student's t-test. (FIG. 15E) Images of harvested tumors on Day 50 post-implantation. Scale bar=1.0 cm. (FIG. 15F) Flow analysis of bone marrow cells derived from one femur. Tumor cells are identified as hCD45⁻ mCD45⁻ GD2⁺ GFP⁺ cells. The total cell number per femur is plotted, and each dot represents one mouse.

### SEQUENCES

The nucleic acid and amino acid sequences listed in the accompanying sequence listing are shown using standard letter abbreviations for nucleotide bases and single letter code for amino acids, as defined in 37 C.F.R. 1.822. Only one strand of each nucleic acid sequence is shown, but the complementary strand is understood as included by any reference to the displayed strand. In the accompanying sequence listing: SEQ ID NO: 1 is a nucleotide sequence encoding the CT3 VH domain.
SEQ ID NO: 2 is the amino acid sequence of the CT3 VH domain.
SEQ ID NO: 3 is a nucleotide sequence encoding the CT3 VL domain.
SEQ ID NO: 4 is the amino acid sequence of the CT3 VL domain.
SEQ ID NO: 5 is a nucleotide sequence encoding the CT3 scFv.
SEQ ID NO: 6 is the amino acid sequence of the CT3 scFv.
SEQ ID NO: 7 is a nucleotide sequence encoding the hCT3-1 (VH-VL) scFv.
SEQ ID NO: 8 is the amino acid sequence of the hCT3-1 (VH-VL) scFv.
SEQ ID NO: 9 is a nucleotide sequence encoding the hCT3-1 (VL-VH) scFv.
SEQ ID NO: 10 is the amino acid sequence of the hCT3-1 (VL-VH) scFv.
SEQ ID NO: 11 is a nucleotide sequence encoding the hCT3-2 (VH-VL) scFv.
SEQ ID NO: 12 is the amino acid sequence of the hCT3-2 (VH-VL) scFv.
SEQ ID NO: 13 is a nucleotide sequence encoding the hCT3-2 (VL-VH) scFv.
SEQ ID NO: 14 is the amino acid sequence of the hCT3-2 (VL-VH) scFv.
SEQ ID NO: 15 is a nucleotide sequence encoding the hCT3-3 (VH-VL) scFv.
SEQ ID NO: 16 is the amino acid sequence of the hCT3-3 (VH-VL) scFv.
SEQ ID NO: 17 is a nucleotide sequence encoding the hCT3-3 (VL-VH) scFv.
SEQ ID NO: 18 is the amino acid sequence of the hCT3-3 (VL-VH) scFv.
SEQ ID NO: 19 is a nucleotide sequence encoding the hCT3-4 (VH-VL) scFv.
SEQ ID NO: 20 is the amino acid sequence of the hCT3-4 (VH-VL) scFv.
SEQ ID NO: 21 is a nucleotide sequence encoding the hCT3-4 (VL-VH) scFv.
SEQ ID NO: 22 is the amino acid sequence of the hCT3-4 (VL-VH) scFv.
SEQ ID NO: 23 is a nucleotide sequence encoding the CD28 hinge region.
SEQ ID NO: 24 is the amino acid sequence of the CD28 hinge region.
SEQ ID NO: 25 is a nucleotide sequence encoding the CD28 transmembrane domain.
SEQ ID NO: 26 is the amino acid sequence of the CD28 transmembrane domain.
SEQ ID NO: 27 is a nucleotide sequence encoding the 4-1BB signaling moiety.
SEQ ID NO: 28 is the amino acid sequence of the 4-1BB signaling moiety.
SEQ ID NO: 29 is a nucleotide sequence encoding the CD3ζ signaling domain.
SEQ ID NO: 30 is the amino acid sequence of the CD3ζ signaling domain.
SEQ ID NO: 31 is a nucleotide sequence encoding the GMCSFR signal sequence.
SEQ ID NO: 32 is the amino acid sequence of the GMCSFR signal sequence.
SEQ ID NO: 33 is a nucleotide sequence encoding the T2A self-cleaving peptide.
SEQ ID NO: 34 is the amino acid sequence of the T2A self-cleaving peptide.
SEQ ID NO: 35 is a nucleotide sequence encoding hEGFRt.
SEQ ID NO: 36 is the amino acid sequence of hEGFRt.
SEQ ID NO: 37 is a nucleotide sequence encoding the CT3.28H.BBz CAR construct.
SEQ ID NO: 38 is the amino acid sequence of the CT3.28H.BBz CAR construct, which includes the following features:
Residues 1-22—GMCSFR signal sequence
Residues 23-24—restriction enzyme site
Residues 25-268—CT3 scFv
Residues 269-270—restriction enzyme site
Residues 271-309—CD28 hinge
Residues 310-336—CD28 transmembrane domain
Residues 337-378-4-1BB co-stimulatory domain
Residues 379-490—CD3ζ signaling domain
Residues 491-508—T2A site
Residues 509-530—GMCSFR signal sequence
Residues 531-865—hEGFRt
SEQ ID NO: 39 is the amino acid sequence of the CT3.8H.BBz CAR construct, which includes the following features:
Residues 1-22—GMCSFR signal sequence
Residues 23-24—restriction enzyme site
Residues 25-268—CT3 scFv
Residues 269-270—restriction enzyme site
Residues 271-315—CD8 hinge
Residues 316-336—CD8 transmembrane domain
Residues 337-378-4-1BB co-stimulatory domain
Residues 379-490—CD3ζ signaling domain
Residues 491-508—T2A site
Residues 509-530—GMCSFR signal sequence
Residues 531-865—hEGFRt
SEQ ID NO: 40 is the amino acid sequence of the CT3.8H.28BBz CAR construct, which includes the following features:
Residues 1-22—GMCSFR signal sequence
Residues 23-24—restriction enzyme site
Residues 25-268—CT3 scFv
Residues 269-270—restriction enzyme site
Residues 271-315—CD8 hinge
Residues 316-342—CD28 transmembrane domain
Residues 343-383—CD28 co-stimulatory domain
Residues 384-425-4-1BB co-stimulatory domain
Residues 426-537—CD3ζ signaling domain
Residues 538-555—T2A site
Residues 556-577—GMCSFR signal sequence
Residues 578-912—hEGFRt

### DETAILED DESCRIPTION

### I. Abbreviations

ALL acute lymphoblastic leukemia
ARMS alveolar rhabdomyosarcoma
BLI bioluminescence imaging
CAR chimeric antigen receptor
CDR complementarity determining region
CNS central nervous system
DRCT desmoplastic small round cell tumor
E:T effector to target
EF1α elongation factor 1 alpha
EGF epidermal growth factor
EGFR epidermal growth factor receptor
ERMS embryonal rhabdomyosarcoma
ffLUC firefly luciferase
GPC2 glypican-2
GMCSFRss granulocyte-macrophage colony stimulating factor receptor signal sequence
hEGFRt human truncated epidermal growth factor receptor
iPSC induced pluripotent stem cell
i.v. intravenous
KO knockout
NB neuroblastoma
NK natural killer
PDX patient-derived xenograft
RMS rhabdomyosarcoma
scFv single-chain variable fragment
TM transmembrane
TME tumor microenvironment
VH variable heavy
UT untransduced
VL variable light
WT wild type

### II. Summary of Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “an antigen” includes singular or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various aspects, the following explanations of terms are provided:

4-1BB: A co-stimulatory molecule expressed by T cell receptor (TCR)-activated lymphocytes, and by other cells including natural killer cells. Ligation of 4-1BB induces a signaling cascade that results in cytokine production, expression of anti-apoptotic molecules and an enhanced immune response. An exemplary amino acid sequence of 4-1BB is set forth herein as SEQ ID NO: 28.

Acute lymphoblastic leukemia (ALL): An acute form of leukemia characterized by the overproduction of lymphoblasts. ALL is most common in childhood, peaking at ages 2-5.

Administration: To provide or give a subject an agent, such as a CAR or CAR-expressing cell provided herein, by any effective route. Exemplary routes of administration include, but are not limited to, injection (such as subcutaneous, intramuscular, intradermal, intraperitoneal, intracerebral, intraventricular, intracranial, intramedullar, intravenous, intra-arterial (including hepatic intra-arterial), intraosseous, intravitreal, and intratumoral), rectal, transdermal, intranasal, vaginal and inhalation routes. In some examples administration is local. In some examples administration is systemic.

Antibody: A polypeptide ligand comprising at least one variable region that recognizes and binds (such as specifically recognizes and specifically binds) an epitope of an antigen, such as GPC2. Mammalian immunoglobulin molecules are composed of a heavy (H) chain and a light (L) chain, each of which has a variable region, termed the variable heavy (V_{H}) region and the variable light (V_{L}) region, respectively. Together, the V_{H} region and the V_{L} region are responsible for binding the antigen recognized by the antibody. There are five main heavy chain classes (or isotypes) of mammalian immunoglobulin, which determine the functional activity of an antibody molecule: IgM, IgD, IgG, IgA and IgE. Some mammals, such as camels, alpacas, and llamas, have heavy-chain antibodies that lack a light chain. Antibody isotypes not found in mammals include IgX, IgY, IgW and IgNAR. IgY is the primary antibody produced by birds and reptiles, and has some functionality similar to mammalian IgG and IgE. IgW and IgNAR antibodies are produced by cartilaginous fish such as sharks, while IgX antibodies are found in amphibians. IgNAR antibodies are heavy-chain antibodies.

Antibody variable regions contain “framework” regions and hypervariable regions, known as “complementarity determining regions” or “CDRs.” The CDRs are primarily responsible for binding to an epitope of an antigen. The framework regions of an antibody serve to position and align the CDRs in three-dimensional space. The amino acid sequence boundaries of a given CDR can be readily determined using any of a number of well-known numbering schemes, including those described by Kabat et al. (Sequences of Proteins of Immunological Interest, U.S. Department of Health and Human Services, 1991; the “Kabat” numbering scheme), Chothia et al. (see Chothia and Lesk, J Mol Biol 196:901-917, 1987; Chothia et al., Nature 342:877, 1989; and Al-Lazikani et al., (JMB 273,927-948, 1997; the “Chothia” numbering scheme), Kunik et al. (see Kunik et al., PLoS Comput Biol 8:e1002388, 2012; and Kunik et al., Nucleic Acids Res 40:W521-524, 2012; “Paratome CDRs”) and the ImMunoGeneTics (IMGT) database (see, Lefranc, Nucleic Acids Res 29:207-9, 2001; the “IMGT” numbering scheme). The Kabat, Paratome and IMGT databases are maintained online.

A “single-domain antibody” refers to an antibody having a single domain (a variable domain) that is capable of specifically binding an antigen, or an epitope of an antigen, in the absence of an additional antibody domain. Single-domain antibodies include, for example, V_{H} domain antibodies, V_{NAR} antibodies, camelid V_{H}H antibodies, and V_{L} domain antibodies. V_{NAR} antibodies are produced by cartilaginous fish, such as nurse sharks, wobbegong sharks, spiny dogfish and bamboo sharks, which produce heavy-chain antibodies (IgNARs). Camelid V_{H}H antibodies are produced by several species including camel, llama, alpaca, and guanaco, which produce heavy chain antibodies that are naturally devoid of light chains.

A “monoclonal antibody” is an antibody produced by a single clone of lymphocytes or by a cell into which the coding sequence of a single antibody has been transfected. Monoclonal antibodies are produced by known methods. Monoclonal antibodies include humanized monoclonal antibodies.

A “chimeric antibody” has framework residues from one species, such as human, and CDRs (which generally confer antigen binding) from another species.

A “humanized” antibody is an immunoglobulin including a human framework region and one or more CDRs from a non-human (for example a mouse, rabbit, rat, shark, camel or synthetic) immunoglobulin. The non-human immunoglobulin providing the CDRs is termed a “donor,” and the human immunoglobulin providing the framework is termed an “acceptor.” In one aspect, all CDRs are from the donor immunoglobulin in a humanized immunoglobulin. Constant regions need not be present, but if they are, they are substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A humanized antibody binds to the same antigen as the donor antibody that provides the CDRs. Humanized or other monoclonal antibodies can have additional conservative amino acid substitutions which have substantially no effect on antigen binding or other immunoglobulin functions.

Binding affinity: Affinity of an antibody or other antigen-binding molecule for an antigen, such as GPC2. In one aspect, affinity is calculated by a modification of the Scatchard method described by Frankel et al., Mol. Immunol., 16:101-106, 1979. In another aspect, binding affinity is measured by an antigen/antibody dissociation rate. In another aspect, a high binding affinity is measured by a competition radioimmunoassay. In another aspect, binding affinity is measured by ELISA. In some aspects, binding affinity is measured using the Octet system (ForteBio), which is based on bio-layer interferometry technology. In other aspects, Kd is measured using surface plasmon resonance assays using, for example, a BIACORES-2000 or a BIACORES-3000 (BIAcore, Inc., Piscataway, N.J.). In other aspects, antibody affinity is measured by flow cytometry. An antibody or CAR that “specifically binds” an antigen (such as GPC2) is an antibody or CAR that binds the antigen with high affinity and does not significantly bind other unrelated antigens.

Chemotherapeutic agent: Any chemical agent with therapeutic usefulness in the treatment of diseases characterized by abnormal cell growth. Such diseases include tumors, neoplasms, and cancer. In one aspect, a chemotherapeutic agent is an agent of use in treating a GPC2-positive tumor. In one aspect, a chemotherapeutic agent is a radioactive compound. Exemplary chemotherapeutic agents that can be used with the methods provided herein are disclosed in Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., © 2000 Churchill Livingstone, Inc; Baltzer, L., Berkery, R. (eds.): Oncology Pocket Guide to Chemotherapy, 2nd ed. St. Louis, Mosby-Year Book, 1995; Fischer, D.S., Knobf, M. F., Durivage, H.J. (eds): The Cancer Chemotherapy Handbook, 4th ed. St. Louis, Mosby-Year Book, 1993). In one example, a chemotherapeutic agent is a biologic, such as a therapeutic antibody (e.g., therapeutic monoclonal antibody), such as an anti-GPC2 antibody, as well as other anti-cancer antibodies, such as anti-PD1 or anti-PDL1 (e.g., pembrolizumab and nivolumab), anti-CTLA4 (e.g., ipilimumab), anti-EGFR (e.g., cetuximab), anti-VEGF (e.g., bevacizumab), or combinations thereof (e.g., anti-PD-1 and anti-CTLA-4). Combination chemotherapy is the administration of more than one agent to treat cancer. One example is the administration of GPC2-targeted CAR-expressing immune cells used in combination with a radioactive, biological, or chemical compound, or combinations thereof.

Chimeric antigen receptor (CAR): A chimeric molecule that includes an antigen-binding portion, such as a single-domain antibody (e.g., V_{NAR}, V_{H}H or V_{H}) or a scFv, and a signaling domain, such as a signaling domain from a T cell receptor (for example, CD3ζ). In many instances, CARs include an antigen-binding moiety, a hinge region, a transmembrane domain and an endodomain. The endodomain can include a signaling chain having an immunoreceptor tyrosine-based activation motif (ITAM), such as CD3ζ or FcεRIγ. In some cases, the endodomain further includes the intracellular portion of at least one additional co-stimulatory domain, such as CD28, 4-1BB (CD137), ICOS, OX40 (CD134), CD27, MYD88-CD40, KIR2DS2 and/or DAP10. In some examples, the CAR is multispecific (such as bispecific) or bicistronic. A multispecific CAR is a single CAR molecule comprised of at least two antigen-binding domains (such as scFvs and/or single-domain antibodies) that each bind a different antigen or a different epitope on the same antigen (see, for example, US 2018/0230225). For example, a bispecific CAR refers to a single CAR molecule having two antigen-binding domains that each bind a different antigen. A bicistronic CAR refers to two complete CAR molecules, each containing an antigen-binding moiety that binds a different antigen. In some cases, a bicistronic CAR construct expresses two complete CAR molecules that are linked by a cleavage linker. Immune cells (such as T cells, NK cells, B cells or macrophages) or iPSCs expressing a bispecific or bicistronic CAR can bind cells that express both of the antigens to which the binding moieties are directed (see, for example, Qin et al., Blood 130:810, 2017; and WO/2018/213337). In some aspects, the CAR is a two-chained antibody-T cell receptor (AbTCR) as described in Xu et al. (Cell Discovery 4:62, 2018) or a synthetic T cell receptor and antigen receptor (STAR) as described by Liu et al. (Sci Transl Med 13(586):eabb5191, 2021).

Complementarity determining region (CDR): A region of hypervariable amino acid sequence that defines the binding affinity and specificity of an antibody. The light and heavy chains of a mammalian immunoglobulin each have three CDRs, designated L-CDR1, L-CDR2, L-CDR3 and H-CDR1, H-CDR2, H-CDR3, respectively. A single-domain antibody contains three CDRs (CDR1, CDR2 and CDR3).

Conservative variant: In the context of the present disclosure, “conservative” amino acid substitutions are those substitutions that do not substantially affect or decrease the affinity of a protein, such as an antibody, to GPC2. As one example, a monoclonal antibody that specifically binds GPC2 can include at most about 1, at most about 2, at most about 5, at most about 10, at most about 15, at most about 20, or at most about 25 conservative substitutions and specifically bind the GPC2 polypeptide. The term “conservative variant” also includes the use of a substituted amino acid in place of an unsubstituted parent amino acid, provided that the variant retains activity. Non-conservative substitutions are those that reduce an activity (such as affinity) of a protein.

Conservative amino acid substitution tables providing functionally similar amino acids are well known. The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

In some aspects herein, provided are amino acid sequences comprising no more than 10, no more than 9, no more than 8, no more than 7, no more than 6, no more than 5, no more than 4, no more than 3, no more than 2 or no more than 1 amino acid substitutions relative to any amino acid sequence disclosed herein.

Contacting: Placement in direct physical association; includes both in solid and liquid form.

Degenerate variant: A polynucleotide encoding a polypeptide that includes a sequence that is degenerate as a result of the genetic code. There are 20 natural amino acids, most of which are specified by more than one codon. Therefore, all degenerate nucleotide sequences are included as long as the amino acid sequence of the polypeptide is unchanged.

Desmoplastic small round cell tumor (DRCT): A soft tissue sarcoma that predominantly occurs in childhood, particularly in boys. DRCT is an aggressive and rare type of cancer that primarily occurs as a mass in the abdomen, but can also be found in the lymph nodes, the lining of the abdomen, diaphragm, spleen, liver, chest wall, skull, spinal cord, intestine, bladder, brain, lungs, testicles, ovaries and the pelvis.

Epitope: An antigenic determinant. These are particular chemical groups or peptide sequences on a molecule that are antigenic (that elicit a specific immune response). An antibody specifically binds a particular antigenic epitope on a polypeptide.

Framework region: Amino acid sequences interposed between CDRs. Framework regions include variable light and variable heavy framework regions. The framework regions serve to hold the CDRs in an appropriate orientation for antigen binding.

Glioma: A type of tumor that occurs in the brain and spinal cord. Gliomas originate in the glial cells that surround and support neurons in the brain, including astrocytes, oligodendrocytes and ependymal cells. There are three classes of gliomas, based on the type of cells from which the tumor arises: astrocytoma, ependymoma, and oligodendroglioma.

Glypican-2 (GPC2): A member of the six-member glypican family of heparan sulfate (HS) proteoglycans that are attached to the cell surface by a GPI anchor (Li et al., Trends Cancer 4(11):741-754, 2018). GPC2 mRNA is highly expressed in neuroblastoma and other pediatric cancers (Orentas et al., Front Oncol 2:194, 2012). GPC2 protein is highly expressed in about half of neuroblastoma cases and the high GPC2 expression correlates with poor overall survival compared with patients with low GPC2 expression (Li et al., Proc Natl Acad Sci USA 114(32):E6623-E6631, 2017). GPC2 is also known as cerebroglycan proteoglycan and glypican proteoglycan 2. GPC2 genomic, mRNA and protein sequences are publicly available (see, for example, NCBI Gene ID 221914).

GPC2-positive cancer: A cancer that expresses or overexpresses GPC2. Examples of GPC2-positive cancers include, but are not limited to, neuroblastoma, medulloblastoma, retinoblastoma, acute lymphoblastic leukemia, embryonal rhabdomyosarcoma, alveolar rhabdomyosarcoma, Ewing's sarcoma, desmoplastic small round cell tumor, glioma and osteosarcoma.

Heterologous: Originating from a separate genetic source or species.

Host cells: Cells in which a vector can be propagated and its DNA expressed. The cell may be prokaryotic or eukaryotic. In some examples, the prokaryotic cell is an E. coli cell. In some examples, the eukaryotic cell is a human cell, such as a human embryonic kidney (HEK) cell. The term also includes any progeny of the subject host cell. It is understood that all progeny may not be identical to the parental cell since there may be mutations that occur during replication. However, such progeny are included when the term “host cell” is used.

Immune response: A response of a cell of the immune system, such as a B cell, T cell, or monocyte, to a stimulus. In one aspect, the response is specific for a particular antigen (an “antigen-specific response”). In one aspect, an immune response is a T cell response, such as a CD4⁺ response or a CD8⁺ response. In another aspect, the response is a B cell response, and results in the production of specific antibodies.

Isolated: An “isolated” biological component, such as a nucleic acid, protein (including antibodies) or organelle, has been substantially separated or purified away from other biological components in the environment (such as a cell) in which the component occurs, e.g., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins.

Label: A detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. In one example, a “labeled antibody” refers to incorporation of another molecule in the antibody. For example, the label is a detectable marker, such as the incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Various methods of labeling polypeptides and glycoproteins are known and may be used. Examples of labels for polypeptides include, but are not limited to, the following: radioisotopes or radionucleotides (such as ³⁵S, ¹¹C, ¹³N, ¹⁵O, ¹⁸F, ¹⁹F, ^{99m}Tc, ¹³¹I, ³H, ¹⁴C, ¹⁵N, ⁹⁰Y ⁹⁹Tc, ¹¹¹In and ¹²⁵I), fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as leucine zipper pair sequences, binding sites for secondary antibodies, metal binding domains, epitope tags), or magnetic agents, such as gadolinium chelates. In some aspects, labels are attached by spacer arms of various lengths to reduce potential steric hindrance.

Linker: In some cases, a linker is a peptide within an antibody binding fragment (such as an scFv fragment) which serves to indirectly bond the variable heavy chain to the variable light chain. In some aspects herein, the disclosed scFv include a (G₄S)₃ linker in different lengths that joins the VH and VL domains of the antigen-binding domain. “Linker” can also refer to a peptide serving to link a targeting moiety, such as an antibody, to an effector molecule, such as a cytotoxin or a detectable label. The terms “conjugating,” “joining,” “bonding” or “linking” refer to making two polypeptides into one contiguous polypeptide molecule, or to covalently attaching a radionuclide or other molecule to a polypeptide, such as an scFv. In the specific context, the terms include reference to joining a ligand, such as an antibody moiety, to an effector molecule. The linkage can be either by chemical or recombinant means. “Chemical means” refers to a reaction between the antibody moiety and the effector molecule such that there is a covalent bond formed between the two molecules to form one molecule.

Mammal: This term includes both human and non-human mammals. Similarly, the term “subject” includes both human and veterinary subjects, such as mice, rats, cows, cats, dogs, pigs, and non-human primates.

Medulloblastoma: A fast-growing type of cancer that forms in the cerebellum. Medulloblastomas tend to spread through the cerebrospinal fluid to the spinal cord or to other parts of the brain. They may also spread to other parts of the body, but this is rare. Medulloblastomas are most common in children and young adults. They are a type of central nervous system embryonal tumor.

Neoplasia, malignancy, cancer or tumor: A neoplasm is an abnormal growth of tissue or cells that results from excessive cell division. Neoplastic growth can produce a tumor. The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.”

Neuroblastoma: A solid tumor arising from embryonic neural crest cells. Neuroblastoma commonly arises in and around the adrenal glands, but can occur anywhere that sympathetic neural tissue is found, such as in the abdomen, chest, neck or nerve tissue near the spine. Neuroblastoma typically occurs in children younger than 5 years of age.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Osteosarcoma: A type of cancerous tumor found in the bone. Osteosarcoma is an aggressive cancer arising from primitive transformed cells of mesenchymal origin. This type of cancer is most prevalent in children and young adults.

Pediatric cancer: A cancer that develops in children ages 0 to 14. The major types of pediatric cancers include, for example, neuroblastoma, acute lymphoblastic leukemia (ALL), embryonal rhabdomyosarcoma (ERMS), alveolar rhabdomyosarcoma (ARMS), Ewing's sarcoma, desmoplastic small round cell tumor (DRCT), osteosarcoma, brain and other CNS tumors (such as medulloblastoma), Wilm's tumor, non-Hodgkin lymphoma, and retinoblastoma.

Pharmaceutically acceptable carriers: The pharmaceutically acceptable carriers of use are conventional. Remington: The Science and Practice of Pharmacy, 22^{nd} ed., London, UK: Pharmaceutical Press, 2013, describes compositions and formulations suitable for pharmaceutical delivery of the CAR-expressing cells and other compositions disclosed herein. The nature of the carrier can depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop, such as a reduction in tumor burden or a decrease in the number or size of metastases. “Ameliorating” refers to the reduction in the number or severity of signs or symptoms of a disease, such as cancer.

Purified: The term purified does not require absolute purity; rather, it is intended as a relative term. Thus, for example, a purified peptide preparation is one in which the peptide or protein is more enriched than the peptide or protein is in its natural environment within a cell. Similarly, a purified cell is one in which the cell is more enriched than the cell is in its natural environment within a subject, or in which the cell is substantially free of other cell types. In one aspect, a preparation is purified such that the protein or peptide represents at least 50% of the total peptide or protein content of the preparation. Substantial purification denotes purification from other proteins or cellular components. A substantially purified protein is at least 60%, 70%, 80%, 90%, 95%, 98%, 99%, 99.9%, or 99.99% pure. Thus, in one specific, non-limiting example, a substantially purified protein is at least 90% free of other proteins or cellular components. A substantially purified cell (such as a cell expressing a CAR provided herein) can be at least 90%, 95%, 98%, 99%, 99.9%, or 99.99% pure. Thus, in one specific, non-limiting example, a substantially purified cell expressing a CAR provided herein is at least 99% free of other cells (such as other immune cells or other cells not expressing a CAR provided herein) or cellular components.

Recombinant: A recombinant nucleic acid is one that has a sequence that is not naturally occurring or has a sequence that is made by an artificial combination of two otherwise separated segments of sequence. This artificial combination is often accomplished by chemical synthesis or by the artificial manipulation of isolated segments of nucleic acids, for example, by genetic engineering techniques.

Retinoblastoma: A type of cancer that forms in the tissues of the retina. Retinoblastoma usually occurs in children younger than 5 years of age. It may be hereditary or nonhereditary (sporadic).

Rhabdomyosarcoma (RMS): A soft tissue malignant tumor of skeletal muscle origin. The most common primary sites for rhabdomyosarcoma are the head and neck (e.g., parameningeal, orbit, pharyngeal, etc.), the genitourinary tract, and the extremities. Other less common primary sites include the trunk, chest wall, the abdomen (including the retroperitoneum and biliary tract), and the perineal/anal region. There are at least two types of RMS; the most common forms are alveolar RMS (ARMS) and embryonal histological RMS (ERMS). Approximately 20% of children with rhabdomyosarcoma have the ARMS subtype. An increased frequency of this subtype is noted in adolescents and in patients with primary sites involving the extremities, trunk, and perineum/perianal region. ARMS is associated with chromosomal translocations encoding a fusion gene involving FKHR on chromosome 13 and members of the PAX family. The embryonal subtype is the most frequently observed subtype in children, accounting for approximately 60-70% of rhabdomyosarcomas of childhood. Tumors with embryonal histology typically arise in the head and neck region or in the genitourinary tract, although they may occur at any primary site. ERMS is characterized by a younger age at diagnosis, loss of heterozygosity, and altered genomic imprinting.

Sample (or biological sample): A biological specimen containing genomic DNA, RNA (including mRNA), protein, or combinations thereof, obtained from a subject. Examples include, but are not limited to, peripheral blood, tissue, cells, urine, saliva, tissue biopsy, fine needle aspirate, surgical specimen, and autopsy material. In one example, a sample includes a tumor biopsy, such as a tumor tissue biopsy.

Sequence identity: The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide or nucleic acid molecule will possess a relatively high degree of sequence identity when aligned using standard methods.

Methods of alignment of sequences for comparison are known. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Homologs and variants of an antibody or CAR that specifically binds GPC2 are typically characterized by possession of at least about 75%, for example at least about 80%, 90%, 95%, 96%, 97%, 98% or 99% sequence identity counted over the full-length alignment with the amino acid sequence of the antibody or CAR using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. These sequence identity ranges are provided for guidance only; it is entirely possible that strongly significant homologs could be obtained that fall outside of the ranges provided.

Subject: Living multi-cellular vertebrate organisms, a category that includes both human and veterinary subjects, including human and non-human mammals such as pigs, mice, rats, rabbits, sheep, horses, cows, dogs, cats and non-human primates.

Synthetic: Produced by artificial means in a laboratory, for example a synthetic nucleic acid or protein (for example, an antibody) can be chemically synthesized in a laboratory.

Therapeutically effective amount: A quantity of a specific substance sufficient to achieve a desired effect in a subject being treated. For instance, this can be the amount necessary to inhibit or suppress growth of a tumor. In one aspect, a therapeutically effective amount is the amount necessary to eliminate, reduce the size, or prevent metastasis of a tumor, such as reduce a tumor size and/or volume by at least 10%, at least 20%, at least 50%, at least 75%, at least 80%, at least 90%, at least 95%, or even 100%, and/or reduce the number and/or size/volume of metastases by at least 10%, at least 20%, at least 50%, at least 75%, at least 80%, at least 90%, at least 95%, or even 100%, for example as compared to a size/volume/number prior to treatment. When administered to a subject, a dosage will generally be used that will achieve target tissue concentrations (for example, in tumors) that has been shown to achieve a desired in vitro effect.

Vector: A nucleic acid molecule as introduced into a host cell, thereby producing a transformed host cell. A vector may include nucleic acid sequences that permit it to replicate in a host cell, such as an origin of replication. A vector may also include one or more selectable marker genes and other genetic elements. In some examples, the vector is a viral vector, such as a lentiviral vector, an adenovirus vector, or an adeno-associated virus (AAV) vector.

### III. Overview of Several Aspects

CAR T cell therapies are a new class of cancer therapeutics being actively developed and tested worldwide. Following the initial success in hematological cancers using CD19-targeted CAR T cells, various CAR strategies have been engineered and tested with the goal of treating solid tumors. However, the success of CAR T cells in solid tumors has been limited by several barriers, including the paucity of tumor-specific antigens, the inability of CAR T cells to efficiently expand at the tumor site, and heterogenous antigen expression (Kochenderfer et al., Blood. 2012; 119(12):2709-2720; Porter et al., N Eng J Med 2011; 365(8):725-733; Jiang et al., Front Immunol 2017; 7:690; Gao et al., Clin Cancer Res 2014; 20(24):6418-6428; Ishiguro et al., Cancer Res 2008; 68(23):9832-9838; Losic et al., Nat Commun 2020; 11(1):291; Li et al., Gastroenterology 158(8):2250-2265, 2020; Li et al., Cell Rep Med 2(6):100297, 2021).

The present disclosure addresses these challenges and improves the efficacy of CAR-expressing cells for treating GPC2-positive tumors. The engineered CARs disclosed herein include an antigen-binding domain derived from GPC2-specific antibody CT3 (PCT Publication No. WO 2020/033430, herein incorporated by reference), or a humanized version thereof. It is disclosed herein that immune cells expressing GPC2-targeted CARs containing a CD28 hinge region and a CD28 transmembrane domain are significantly more potent at killing GPC2-positive tumors compared to GPC2-targeted CARs containing a CD8 hinge region and either a CD8 or CD28 transmembrane domain. Furthermore, using orthotopic neuroblastoma models, it was demonstrated that immune cells expressing GPC2-targeted CARs having a CD28 hinge and transmembrane domain exhibited superior expansion in vitro and in vivo against GPC2-positive tumors cells, resulted in higher levels of tumor-infiltrating CAR+ T cells, and led to increased survival, relative to GPC2-targeted CARs having a CD8 hinge and transmembrane domain. Moreover, immune cells expressing GPC2-targeted CARs having a CD28 hinge and transmembrane domain showed superior antitumor activity in a neuroblastoma model compared to an existing CAR T cell therapy for neuroblastoma.

Provided herein are CARs that include an extracellular antigen-binding domain that specifically binds GPC2; a CD28 hinge region; a CD28 transmembrane domain; an intracellular co-stimulatory domain; and an intracellular signaling domain. In some aspects, the GPC2-specific antigen-binding domain is a scFv. The scFv can have an N-terminal to C-terminal orientation of VH-linker-VL, or VL-linker-VH.

In some aspects, the antigen-binding domain includes a variable heavy (VH) domain and a variable light (VL) domain, wherein the VH domain includes the complementarity determining region 1 (CDR1), CDR2 and CDR3 sequences of SEQ ID NO: 2 (the CT3 VH domain sequence) and/or the VL domain includes the CDR1, CDR2 and CDR3 sequences of SEQ ID NO: 4 (the CT3 VL domain sequence). In some examples, the CDR sequences are defined using the Kabat, IMGT or Paratome numbering schemes, or a combination of the Kabat, IMGT and Paratome numbering schemes. In other examples, the CDR sequences are determined using a different numbering scheme, such as Chothia.

In some aspects, the VH domain CDR1, CDR2 and CDR3 sequences respectively include residues 31-35, 50-66 and 99-112 of SEQ ID NO: 2 and/or the VL domain CDR1, CDR2 and CDR3 sequences respectively include residues 24-33, 49-55 and 88-96 of SEQ ID NO: 4; the VH domain CDR1, CDR2 and CDR3 sequences respectively include residues 26-33, 51-58 and 97-112 of SEQ ID NO: 2 and/or the VL domain CDR1, CDR2 and CDR3 sequences respectively include residues 27-31, 49-51 and 88-96 of SEQ ID NO: 4; the VH domain CDR1, CDR2 and CDR3 sequences respectively include residues 26-35, 47-61 and 97-112 of SEQ ID NO: 2 and/or the VL domain CDR1, CDR2 and CDR3 sequences respectively include residues 27-33, 45-55 and 88-95 of SEQ ID NO: 4; or the VH domain CDR1, CDR2 and CDR3 sequences respectively include residues 26-35, 47-66 and 97-112 of SEQ ID NO: 2 and/or the VL domain CDR1, CDR2 and CDR3 sequences respectively include residues 24-33, 45-55 and 88-96 of SEQ ID NO: 4. In some examples, the amino acid sequence of the VH domain is at least 80%, at least 85%, at least 90%, at least 95%, at least 96%, at least 97%, at least 98%, at least 99%, or 100% identical to SEQ ID NO: 2 (and includes the CDR1, CDR2 and CDR3 sequences of SEQ ID NO: 2) and/or the amino acid sequence of the VL domain is at least 80%, at least 85%, at least 90%, at least 95%, at least 96%, at least 97%, at least 98%, at least 99%, or 100% identical to SEQ ID NO: 4 (and includes the CDR1, CDR2 and CDR3 sequences of SEQ ID NO: 4).

In some aspects, the VH domain and the VL domain sequences are humanized. In some examples, the amino acid sequence of the humanized VH domain includes residues 1-123 of SEQ ID NO: 8, and/or the amino acid sequence of the humanized VL domain includes residues 139-244 of SEQ ID NO: 8; the amino acid sequence of the humanized VH domain includes residues 1-122 of SEQ ID NO: 12, and/or the amino acid sequence of the humanized VL domain includes residues 138-243 of SEQ ID NO: 12; the amino acid sequence of the humanized VH domain includes residues 1-122 of SEQ ID NO: 16, and/or the amino acid sequence of the humanized VL domain includes residues 138-244 of SEQ ID NO: 16; or the amino acid sequence of the humanized VH domain includes residues 1-122 of SEQ ID NO: 20, and/or the amino acid sequence of the humanized VL domain includes residues 138-243 of SEQ ID NO: 20.

In some aspects, the amino acid sequence of the extracellular antigen-binding domain is at least 80%, at least 85%, at least 90%, at least 95%, at least 96%, at least 97%, at least 98%, at least 99%, or 100% identical to SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20 or SEQ ID NO: 22. In some examples, the amino acid sequence of the antigen-binding domain includes or consists of the amino acid sequence of SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20 or SEQ ID NO: 22.

In some aspects, the amino acid sequence of the CD28 hinge region is at least 80%, at least 85%, at least 90%, at least 95%, at least 96%, at least 97%, at least 98%, at least 99%, or 100% identical to the amino acid sequence of SEQ ID NO: 24. In some examples, the amino acid sequence of the CD28 hinge region includes or consists of the amino acid sequence of SEQ ID NO: 24.

In some aspects, the amino acid sequence of the CD28 transmembrane domain is at least 80%, at least 85%, at least 90%, at least 95%, at least 96%, at least 97%, at least 98%, at least 99%, or 100% identical to the amino acid sequence of SEQ ID NO: 26. In some examples, the amino acid sequence of the CD28 transmembrane domain includes or consists of the amino acid sequence of SEQ ID NO: 26.

In some aspects, the co-stimulatory domain includes a 4-1BB signaling moiety. In some examples, the amino acid sequence of the 4-1BB signaling moiety is at least 80%, at least 85%, at least 90%, at least 95%, at least 96%, at least 97%, at least 98%, at least 99%, or 100% identical to SEQ ID NO: 28. In specific examples, the amino acid sequence of the 4-1BB signaling moiety includes or consists of the amino acid sequence of SEQ ID NO: 28.

In some aspects, the signaling domain includes a CD3ζ signaling domain. In some examples, the amino acid sequence of the CD3ζ signaling domain is at least 80%, at least 85%, at least 90%, at least 95%, at least 96%, at least 97%, at least 98%, at least 99%, or 100% identical to the amino acid sequence of SEQ ID NO: 30. In specific examples, the amino acid sequence of the CD3ζ signaling domain includes or consists of SEQ ID NO: 30.

In particular aspects, the amino acid sequence of the CAR is at least 80%, at least 85%, at least 90%, at least 95%, at least 96%, at least 97%, at least 98%, at least 99%, or 100% identical to the amino acid sequence of SEQ ID NO: 38. In specific examples, the amino acid sequence of the CAR includes or consists of the amino acid sequence of SEQ ID NO: 38.

In alternative aspects, the amino acid sequence of the CAR is at least 80%, at least 85%, at least 90%, at least 95%, at least 96%, at least 97%, at least 98%, at least 99%, or 100% identical to the amino acid sequence of SEQ ID NO: 39 or SEQ ID NO: 40. In specific examples, the amino acid sequence of the CAR includes or consists of the amino acid sequence of SEQ ID NO: 39 or SEQ ID NO: 40.

Further provided herein are nucleic acid molecules that encode a CAR disclosed herein. In some aspects, the sequence of the nucleic acid molecule is at least 80%, at least 85%, at least 90%, at least 95%, at least 96%, at least 97%, at least 98%, at least 99%, or 100% identical to nucleotides 73-1470 of SEQ ID NO: 37. In some examples, the sequence of the nucleic acid molecule includes or consists of nucleotides 73-1470 of SEQ ID NO: 37. In specific non-limiting examples, the sequence of the nucleic acid molecule includes or consists of SEQ ID NO: 37.

In some aspects, the nucleic acid molecule is operably linked to a promoter (such as an inducible or constitutive promoter). In some examples, the promoter is the human elongation factor 1α (EF1α) promoter.

In some aspects, the nucleic acid molecule includes, in the 5′ to 3′ direction, a nucleic acid encoding a first granulocyte-macrophage colony stimulating factor receptor signal sequence (GMCSFRss); a nucleic acid encoding the antigen-binding domain; a nucleic acid encoding the CD28 hinge region; a nucleic acid encoding the CD28 transmembrane domain; a nucleic acid encoding the co-stimulatory domain; a nucleic acid encoding the signaling domain; a nucleic acid encoding a self-cleaving 2A peptide; a nucleic acid encoding a second GMCSFRss; and a nucleic acid encoding a truncated human epidermal growth factor receptor (hEGFRt). In some examples, the nucleic acid molecule further includes a human elongation factor 1α (EF1α) promoter sequence 5′ of the nucleic acid encoding the first GMCSFRss (see WO 2019/094482, which is herein incorporated by reference in its entirety).

Vectors that include a nucleic acid molecule disclosed herein are further provided. In some examples, the vector is a viral vector, such as a lentiviral vector, an adenovirus vector or an adeno-associated virus vector.

Also provided are isolated cells that include a nucleic acid molecule (or vector) encoding a CAR disclosed herein and/or that express a CAR disclosed herein. In some aspects, the cell is an immune cell, such as a T cell, NK cell, B cell or macrophage. In other aspects, the cell is an induced pluripotent stem cell (iPSC).

Further provided are compositions that include a pharmaceutically acceptable carrier (such as water or saline) and a CAR, nucleic acid molecule, vector, or cell disclosed herein. In some examples, the composition is frozen. In some examples, the composition is frozen and includes cells and DMSO or another cryopreservative. In some examples, the composition is lyophilized. In some examples, such compositions are in a vial, such as a glass or plastic vial. The disclosed compositions can be part of a kit, such as one that includes one or more chemotherapeutic agents, a syringe, cell culture media, pharmaceutically acceptable carrier or combinations thereof (wherein the additional agents in the kit may be in separate containers).

Methods of treating a GPC2-positive cancer, or inhibiting tumor growth or metastasis of a GPC2-positive cancer, in a subject are also provided. In some aspects, the methods include administering to the subject a therapeutically effective amount of a CAR, nucleic acid molecule, vector, cell or composition disclosed herein. In some examples, the GPC2-positive cancer is a solid tumor. In some examples, the GPC2-positive cancer is a pediatric cancer. In particular non-limiting examples, the GPC2-positive cancer is a neuroblastoma, medulloblastoma, retinoblastoma, acute lymphoblastic leukemia, embryonal rhabdomyosarcoma, alveolar rhabdomyosarcoma, Ewing's sarcoma, desmoplastic small round cell tumor, glioma or osteosarcoma. In specific examples, the GPC2-positive cancer is neuroblastoma. In some examples, the methods further include administering conditioning chemotherapy to the subject, such as fludarabine and cyclophosphamide.

### IV. GPC2-Specific Antibody Sequences

The CARs disclosed herein include an antibody (or antigen-binding fragment thereof) that specifically binds GPC2. In some aspects, the antibody is CT3, a murine monoclonal antibody, or a humanized version thereof (e.g., hCT3-1, hCT3-2, hCT3-3 or hCT3-4), in scFv format. These antibodies are described in PCT Publication No. WO 2020/033430, which is herein incorporated by reference in its entirety. The nucleotide and amino acid sequences of CT3 are provided below. Tables 1 and 2 list the amino acid positions of the CDR1, CDR2 and CDR3 of the VH domain and VL domain, respectively, as determined using Kabat, IMGT, Paratome and the combination thereof. The CDR boundaries can also be defined using an alternative numbering scheme, such as the Chothia numbering scheme. The scFv nucleotide and amino acid sequences of the parental CT3 antibody, as well as four humanized versions thereof, are also listed below. In each scFv sequence, the VH and VL domains are separated by a (G₄S)₃ linker, which is shown in bold font. For the humanized antibodies, scFv sequences in the VH-linker-VL orientation and the VL-linker-VH orientation are provided.

CT3 VH nucleotide sequence
(SEQ ID NO: 1)
GAGGTCCAGCTGCAACAGTCTGGACCTGAACTGGTGAAGCCTGGGGCTT
CAGTAAAGATGTCCTGCAAGGCTTCTAGATTCACATTCACTGACTACAA
CATACACTGGGTGAAGCAGAGCCCTGGAAAGACCCTTGAATGGATTGGA
TATATTAACCCTAACAATGGTGATATTTTCTACAAACAGAAGTTCAATG
GCAAGGCCACATTGACTATAAACAAGTCCTCCAACACAGCCTACATGGA
GCTCCGCAGCCTGACATCGGAGGATTCTGCAGTCTATTACTGTGTAAGA
TCCTCTAATATTCGTTATACTTTCGACAGGTTCTTCGATGTCTGGGGCA
CAGGGACCACGGTCACCGTCTCCTCA
CT3 VH amino acid sequence
(SEQ ID NO: 2)
EVQLQQSGPELVKPGASVKMSCKASRFTFTDYNIHWVKQSPGKTLEWIG
YINPNNGDIFYKQKFNGKATLTINKSSNTAYMELRSLTSEDSAVYYCVR
SSNIRYTFDRFFDVWGTGTTVTVSS

TABLE 1
Locations of the CDRs in the CT3 VH domain (SEQ ID NO: 2)

| Scheme | CDR1 | CDR2 | CDR3 |
|---|---|---|---|
| Kabat | 31-35 | 50-66 | 99-112 |
| IMGT | 26-33 | 51-58 | 97-112 |
| Paratome | 26-35 | 47-61 | 97-112 |
| Combined | 26-35 | 47-66 | 97-112 |

CT3 VL nucleotide sequence
(SEQ ID NO: 3)
GAAAATGTGCTCACCCAGTCTCCAGCAATCATGTCTGCATCTCTAGGGG
AGAAGGTCACCATGAGCTGCAGGGCCAGCTCAAGTGTAAATTACATTTA
CTGGTACCAGCAGAAGTCAGATGCCTCCCCCAAACTATGGATTTATTAC
ACATCCAACCTGGCTCCTGGAGTCCCAGCTCGCTTCAGTGGCAGTGGGT
CTGGGAACTCTTATTCTCTCACAATCAGCAGCATGGAGGGTGAAGATGC
TGCCACTTATTACTGCCAGCAGTTTTCTAGTTCCCCATCCACGTTCGGT
ACTGGGACCAAGCTGGAGCTGAAA
CT3 VL amino acid sequence
(SEQ ID NO: 4)
ENVLTQSPAIMSASLGEKVTMSCRASSSVNYIYWYQQKSDASPKLWIYY
TSNLAPGVPARFSGSGSGNSYSLTISSMEGEDAATYYCQQFSSSPSTFG
TGTKLELK

TABLE 2
Locations of the CDRs in the CT3 VL domain (SEQ ID NO: 4)

| Scheme | CDR1 | CDR2 | CDR3 |
|---|---|---|---|
| Kabat | 24-33 | 49-55 | 88-96 |
| IMGT | 27-31 | 49-51 | 88-96 |
| Paratome | 27-33 | 45-55 | 88-95 |
| Combined | 24-33 | 45-55 | 88-96 |

CT3 (VH-VL) scFv nucleotide sequence
(SEQ ID NO: 5)
GAGGTCCAGCTGCAACAGTCTGGACCTGAACTGGTGAAGCCTGGGGCTTCAGTAAAGATGTCC
TGCAAGGCTTCTAGATTCACATTCACTGACTACAACATACACTGGGTGAAGCAGAGCCCTGGA
AAGACCCTTGAATGGATTGGATATATTAACCCTAACAATGGTGATATTTTCTACAAACAGAAGT
TCAATGGCAAGGCCACATTGACTATAAACAAGTCCTCCAACACAGCCTACATGGAGCTCCGCA
GCCTGACATCGGAGGATTCTGCAGTCTATTACTGTGTAAGATCCTCTAATATTCGTTATACTTTC
GACAGGTTCTTCGATGTCTGGGGCACAGGGACCACGGTCACCGTCTCCTCAGGCGGAGGCGG
ATCAGGTGGTGGCGGATCTGGAGGTGGCGGAAGCGAAAATGTGCTCACCCAGTCTCCAGCA
ATCATGTCTGCATCTCTAGGGGAGAAGGTCACCATGAGCTGCAGGGCCAGCTCAAGTGTAAAT
TACATTTACTGGTACCAGCAGAAGTCAGATGCCTCCCCCAAACTATGGATTTATTACACATCCA
ACCTGGCTCCTGGAGTCCCAGCTCGCTTCAGTGGCAGTGGGTCTGGGAACTCTTATTCTCTCAC
AATCAGCAGCATGGAGGGTGAAGATGCTGCCACTTATTACTGCCAGCAGTTTTCTAGTTCCCCA
TCCACGTTCGGTACTGGGACCAAGCTGGAGCTGAAA
CT3 (VH-VL) scFv amino acid sequence
(SEQ ID NO: 6)
EVQLQQSGPEL VKPGASVKMSCKASRFTFTDYNIHWVKQSPGKTLEWIGYINPNNGDIFYKQKFNG
KATLTINKSSNTAYMELRSLTSEDSAVYYCVRSSNIRYTFDRFFDVWGTGTTVTVSSGGGGSGGG
GSGGGGSENVLTQSPAIMSASLGEKVTMSCRASSSVNYIYWYQQKSDASPKLWIYYTSNLAPGVP
ARFSGSGSGNSYSLTISSMEGEDAATYYCQQFSSSPSTFGTGTKLELK
hCT3-1 (VH-VL) nucleotide sequence
(SEQ ID NO: 7)
CAAGTACAGCTTGTACAATCCGGAGCGGAAGTAAAGAAGCCCGGGGCCTCCGTGAAAGTCAGC
TGCAAAGCGTCTAGATTCACCTTCACTGACTATAACATCCACTGGGTGCGGCAAGCGCCTGGTC
AGGGCCTCGAATGGATTGGCTATATAAACCCGAACAACGGGGACATTTTCTATAAGCAGAAAT
TCAATGGCAGAGTGACGCTCACCGCGGACAAAAGTACCAGTACTGCTTATATGGAACTGTCTA
GTCTTACGAGTGAGGATACCGCTGTGTATTATTGCGTGAGGTCCTCCAATATACGCTATACGTT
TGATAGATTCTTTGATGTATGGGGACAGGGAACCCTCGTCACGGTCAGCTCAGGCGGAGGCG
GATCAGGTGGTGGCGGATCTGGAGGTGGCGGAAGCGACGTAGTAATGACTCAAAGCCCCC
TCTCCTTGCCAGTGACGCCAGGTGAGCCGGCATCTATTTCTTGCAGGGCTAGTTCTTCAGTGAA
CTACATCTATTGGTATTTGCAGAAGCCAGGACAAAGTCCGCAGCTTTGGATATATTACACGAGC
AACCTTGCGCCAGGTGTTCCAGATCGCTTCTCTGGCAGTGGATCAGGGACGGACTTCACACTGA
AGATCTCTCGCGTTGAAGCTGAAGACGTCGGCGTGTACTATTGCCAACAATTCAGTAGTTCTCC
GAGCACTTTTGGCCAGGGAACCAAGCTTGAGATTAAA
hCT3-1 (VH-VL) amino acid sequence
(SEQ ID NO: 8)
QVQLVQSGAEVKKPGASVKVSCKASRFTFTDYNIHWVRQAPGQGLEWIGYINPNNGDIFYKQKFN
GRVTLTADKSTSTAYMELSSLTSEDTAVYYCVRSSNIRYTFDRFFDVWGQGTLVTVSSGGGGSGG
GGSGGGGSDVVMTQSPLSLPVTPGEPASISCRASSSVNYIYWYLQKPGQSPQLWIYYTSNLAPGVP
DRFSGSGSGTDFTLKISRVEAEDVGVYYCQQFSSSPSTFGQGTKLEIK
hCT3-1 (VL-VH) nucleotide sequence
(SEQ ID NO: 9)
GACGTAGTAATGACTCAAAGCCCCCTCTCCTTGCCAGTGACGCCAGGTGAGCCGGCATCTATTT
CTTGCAGGGCTAGTTCTTCAGTGAACTACATCTATTGGTATTTGCAGAAGCCAGGACAAAGTCC
GCAGCTTTGGATATATTACACGAGCAACCTTGCGCCAGGTGTTCCAGATCGCTTCTCTGGCAGT
GGATCAGGGACGGACTTCACACTGAAGATCTCTCGCGTTGAAGCTGAAGACGTCGGCGTGTAC
TATTGCCAACAATTCAGTAGTTCTCCGAGCACTTTTGGCCAGGGAACCAAGCTTGAGATTAAAG
GCGGAGGCGGATCAGGTGGTGGCGGATCTGGAGGTGGCGGAAGCCAAGTACAGCTTGTA
CAATCCGGAGCGGAAGTAAAGAAGCCCGGGGCCTCCGTGAAAGTCAGCTGCAAAGCGTCTAG
ATTCACCTTCACTGACTATAACATCCACTGGGTGCGGCAAGCGCCTGGTCAGGGCCTCGAATGG
ATTGGCTATATAAACCCGAACAACGGGGACATTTTCTATAAGCAGAAATTCAATGGCAGAGTG
ACGCTCACCGCGGACAAAAGTACCAGTACTGCTTATATGGAACTGTCTAGTCTTACGAGTGAG
GATACCGCTGTGTATTATTGCGTGAGGTCCTCCAATATACGCTATACGTTTGATAGATTCTTTGA
TGTATGGGGACAGGGAACCCTCGTCACGGTCAGCTCA
hCT3-1 (VL-VH) amino acid sequence
(SEQ ID NO: 10)
DVVMTQSPLSLPVTPGEPASISCRASSSVNYIYWYLQKPGQSPQLWIYYTSNLAPGVPDRFSGSGSG
TDFTLKISRVEAEDVGVYYCQQFSSSPSTFGQGTKLEIKGGGGGGGGSGGGGSQVQLVQSGAEV
KKPGASVKVSCKASRFTFTDYNIHWVRQAPGQGLEWIGYINPNNGDIFYKQKFNGRVTLTADKSTS
TAYMELSSLTSEDTAVYYCVRSSNIRYTFDRFFDVWGQGTLVTVSS
hCT3-2 (VH-VL) nucleotide sequence
(SEQ ID NO: 11)
CAGGTCCAGCTTGTCCAGTCTGGTGCTGAGGTTAAAAAACCTGGCGCAAGCGTGAAGGTGAGC
TGTAAGGCTAGCCGATTTACCTTTACCGACTACAACATACATTGGGTCAGGCAAGCGCCTGGAC
AAAGGCTCGAGTGGATTGGTTACATAAACCCAAATAACGGGGACATATTTTACAAACAGAAAT
TCAACGGCCGGGTAACTATAACACGAGACACAAGTGCAAGTACCGCCTATATGGAGCTCTCTT
CCCTTCGATCCGAAGACACGGCAGTCTACTACTGCGTTCGCAGCTCCAATATCCGCTATACCTT
TGATAGGTTCTTTGATGTGTGGGGTCAAGGGACGCTCGTAACCGTGAGCGGAGGAGGCGGTT
CTGGTGGCGGGGGCAGCGGTGGGGGAGGGTCTGATGTCGTTATGACTCAGAGTCCAGCGTT
TCTCAGTGTTACACCCGGTGAAAAGGTCACCATTACGTGCCGGGCGAGCTCTTCAGTTAACTAC
ATTTATTGGTACCAGCAGAAGCCAGATCAGGCGCCCAAACTTTGGATTTACTATACCAGCAACC
TTGCACCCGGTGTACCCAGTCGGTTTAGCGGCAGTGGGAGTGGTACTGACTTCACTTTTACTAT
TTCATCATTGGAGGCTGAAGATGCTGCGACATACTATTGTCAGCAATTCAGCTCCTCTCCCAGC
ACCTTTGGCCAGGGCACCAAACTTGAAATTAAA
hCT3-2 (VH-VL) amino acid sequence
(SEQ ID NO: 12)
QVQLVQSGAEVKKPGASVKVSCKASRFTFTDYNIHWVRQAPGQRLEWIGYINPNNGDIFYKQKFN
GRVTITRDTSASTAYMELSSLRSEDTAVYYCVRSSNIRYTFDRFFDVWGQGTLVTVSGGGGSGGG
GSGGGGSDVVMTQSPAFLSVTPGEKVTITCRASSSVNYIYWYQQKPDQAPKLWIYYTSNLAPGVPS
RFSGSGSGTDFTFTISSLEAEDAATYYCQQFSSSPSTFGQGTKLEIK
hCT3-2 (VL-VH) nucleotide sequence
(SEQ ID NO: 13)
GATGTCGTTATGACTCAGAGTCCAGCGTTTCTCAGTGTTACACCCGGTGAAAAGGTCACCATTA
CGTGCCGGGCGAGCTCTTCAGTTAACTACATTTATTGGTACCAGCAGAAGCCAGATCAGGCGC
CCAAACTTTGGATTTACTATACCAGCAACCTTGCACCCGGTGTACCCAGTCGGTTTAGCGGCAG
TGGGAGTGGTACTGACTTCACTTTTACTATTTCATCATTGGAGGCTGAAGATGCTGCGACATAC
TATTGTCAGCAATTCAGCTCCTCTCCCAGCACCTTTGGCCAGGGCACCAAACTTGAAATTAAAG
GAGGAGGCGGTTCTGGTGGCGGGGGCAGCGGTGGGGGAGGGTCTCAGGTCCAGCTTGTC
CAGTCTGGTGCTGAGGTTAAAAAACCTGGCGCAAGCGTGAAGGTGAGCTGTAAGGCTAGCCGA
TTTACCTTTACCGACTACAACATACATTGGGTCAGGCAAGCGCCTGGACAAAGGCTCGAGTGG
ATTGGTTACATAAACCCAAATAACGGGGACATATTTTACAAACAGAAATTCAACGGCCGGGTA
ACTATAACACGAGACACAAGTGCAAGTACCGCCTATATGGAGCTCTCTTCCCTTCGATCCGAAG
ACACGGCAGTCTACTACTGCGTTCGCAGCTCCAATATCCGCTATACCTTTGATAGGTTCTTTGAT
GTGTGGGGTCAAGGGACGCTCGTAACCGTGAGC
hCT3-2 (VL-VH) nucleotide sequence
(SEQ ID NO: 14)
DVVMTQSPAFLSVTPGEKVTITCRASSSVNYIYWYQQKPDQAPKLWIYYTSNLAPGVPSRFSGSGS
GTDFTFTISSLEAEDAATYYCQQFSSSPSTFGQGTKLEIKGGGGSGGGGSGGGGSQVQLVQSGAEV
KKPGASVKVSCKASRFTFTDYNIHWVRQAPGQRLEWIGYINPNNGDIFYKQKFNGRVTITRDTSAS
TAYMELSSLRSEDTAVYYCVRSSNIRYTFDRFFDVWGQGTLVTVS
hCT3-3 (VH-VL) nucleotide sequence
(SEQ ID NO: 15)
CAAGTGCAGTTGGTGCAATCAGGAGCGGAAGTCAAGAAACCGGGGGCATCTGTCAAAGTGAG
CTGCAAGGCGAGCCGGTTCACATTTACGGATTACAACATACATTGGGTGCGCCAAGCCCCGGG
ACAGGGCCTCGAATGGATAGGCTACATCAATCCTAATAATGGGGATATCTTCTATAAGCAGAA
ATTTAATGGAAAGGCAACCATGACAGTAGATACTTCTACTAGCACAGTTTACATGGAGCTGTCC
TCACTGCGGTCTGAGGATACGGCGGTGTACTATTGCGTTAGGAGCAGCAATATACGGTACACG
TTCGATCGCTTCTTCGATGTTTGGGGCCAGGGCACCCTCGTGACGGTATCTGGAGGAGGCGGG
TCAGGTGGCGGTGGCTCAGGCGGGGGGGGGAGTATGGACATCCAGATGACCCAGAGCCCT
AGCAGTTTGTCAGCTTCCGTGGGAGACAGAGTCACCATAACATGTCGAGCATCCAGTAGTGTC
AACTATATATACTGGTATCAACAGAAGTCCGGCAAGGCGCCTAAACTGTGGATTTATTATACGT
CCAACCTCGCACCCGGCGTTCCAAGCCGATTCTCCGGCAGTGGATCAGGAACCGACTTCACGCT
GACGATCAGTAGTCTTCAGCCAGAGGATTTCGCTACATATTACTGTCAACAGTTTTCCTCAAGC
CCTAGTACCTTTGGGCAGGGAACCAAACTGGAGATAAAG
hCT3-3 (VH-VL) amino acid sequence
(SEQ ID NO: 16)
QVQLVQSGAEVKKPGASVKVSCKASRFTFTDYNIHWVRQAPGQGLEWIGYINPNNGDIFYKQKFN
GKATMTVDTSTSTVYMELSSLRSEDTAVYYCVRSSNIRYTFDRFFDVWGQGTLVTVSGGGGSGG
GGSGGGGSMDIQMTQSPSSLSASVGDRVTITCRASSSVNYIYWYQQKSGKAPKLWIYYTSNLAPG
VPSRFSGSGSGTDFTLTISSLQPEDFATYYCQQFSSSPSTFGQGTKLEIK
hCT3-3 (VL-VH) nucleotide sequence
(SEQ ID NO: 17)
ATGGACATCCAGATGACCCAGAGCCCTAGCAGTTTGTCAGCTTCCGTGGGAGACAGAGTCACC
ATAACATGTCGAGCATCCAGTAGTGTCAACTATATATACTGGTATCAACAGAAGTCCGGCAAG
GCGCCTAAACTGTGGATTTATTATACGTCCAACCTCGCACCCGGCGTTCCAAGCCGATTCTCCG
GCAGTGGATCAGGAACCGACTTCACGCTGACGATCAGTAGTCTTCAGCCAGAGGATTTCGCTA
CATATTACTGTCAACAGTTTTCCTCAAGCCCTAGTACCTTTGGGCAGGGAACCAAACTGGAGAT
AAAGGGAGGAGGCGGGTCAGGTGGCGGTGGCTCAGGCGGGGGGGGGAGTCAAGTGCAGT
TGGTGCAATCAGGAGCGGAAGTCAAGAAACCGGGGGCATCTGTCAAAGTGAGCTGCAAGGCG
AGCCGGTTCACATTTACGGATTACAACATACATTGGGTGCGCCAAGCCCCGGGACAGGGCCTC
GAATGGATAGGCTACATCAATCCTAATAATGGGGATATCTTCTATAAGCAGAAATTTAATGGA
AAGGCAACCATGACAGTAGATACTTCTACTAGCACAGTTTACATGGAGCTGTCCTCACTGCGGT
CTGAGGATACGGCGGTGTACTATTGCGTTAGGAGCAGCAATATACGGTACACGTTCGATCGCTT
CTTCGATGTTTGGGGCCAGGGCACCCTCGTGACGGTATCT
hCT3-3 (VL-VH) amino acid sequence
(SEQ ID NO: 18)
MDIQMTQSPSSLSASVGDRVTITCRASSSVNYIYWYQQKSGKAPKLWIYYTSNLAPGVPSRFSGSGS
GTDFTLTISSLQPEDFATYYCQQFSSSPSTFGQGTKLEIKGGGGSGGGGSGGGGSQVQLVQSGAEV
KKPGASVKVSCKASRFTFTDYNIHWVRQAPGQGLEWIGYINPNNGDIFYKQKFNGKATMTVDTST
STVYMELSSLRSEDTAVYYCVRSSNIRYTFDRFFDVWGQGTLVTVS
hCT3-4 (VH-VL) nucleotide sequence
(SEQ ID NO: 19)
CAGGTGCAGCTGGTCCAGAGTGGGGCAGAAGTCAAGAAACCCGGAGCAAGTGTCAAAGTGTC
CTGTAAAGCCTCACGATTCACATTTACCGACTATAACATCCACTGGGTGAGACAGGCACCAGG
ACAGAGGCTGGAGTGGATCGGCTATATCAACCCTAACAATGGCGACATCTTCTACAAGCAGAA
GTTTAATGGCCGGGTGACCATCACAAGAGATACCAGCGCCTCCACAGCCTATATGGAGCTGAG
CTCCCTGCGGAGCGAGGATACCGCCGTGTACTATTGCGTGAGGTCTAGCAATATCCGCTACACA
TTCGACCGGTTCTTTGACGTGTGGGGCCAGGGCACCCTGGTGACAGTGTCCGGCGGCGGCGG
CTCTGGCGGAGGAGGCAGCGGCGGAGGAGGCTCCGAGATCGTGCTGACCCAGTCTCCTGCC
ACACTGTCTCTGAGCCCAGGAGAGAGGGCCACCCTGTCCTGTAGGGCCTCCTCTAGCGTGAACT
ACATCTATTGGTACCAGCAGAAGCCAGGCCAGGCCCCCAGACTGTGGATCTACTATACCTCCA
ATCTGGCACCAGGCATCCCTGCAAGGTTCTCCGGCTCTGGCAGCGGCACAGACTTTACCCTGAC
AATCTCCTCTCTGGAGCCCGAGGATTTTGCCGTGTACTATTGTCAGCAGTTTAGCAGTTCACCA
AGCACATTCGGGCAGGGCACCAAGCTGGAAATCAAG
hCT3-4 (VH-VL) amino acid sequence
(SEQ ID NO: 20)
QVQLVQSGAEVKKPGASVKVSCKASRFTFTDYNIHWVRQAPGQRLEWIGYINPNNGDIFYKQKFN
GRVTITRDTSASTAYMELSSLRSEDTAVYYCVRSSNIRYTFDRFFDVWGQGTLVTVSGGGGSGGG
GSGGGGSEIVLTQSPATLSLSPGERATLSCRASSSVNYIYWYQQKPGQAPRLWIYYTSNLAPGIPAR
FSGSGSGTDFTLTISSLEPEDFAVYYCQQFSSSPSTFGQGTKLEIK
hCT3-4 (VL-VH) nucleotide sequence
(SEQ ID NO: 21)
GAGATCGTGCTGACCCAGTCTCCTGCCACACTGTCTCTGAGCCCAGGAGAGAGGGCCACCCTG
TCCTGTAGGGCCTCCTCTAGCGTGAACTACATCTATTGGTACCAGCAGAAGCCAGGCCAGGCCC
CCAGACTGTGGATCTACTATACCTCCAATCTGGCACCAGGCATCCCTGCAAGGTTCTCCGGCTC
TGGCAGCGGCACAGACTTTACCCTGACAATCTCCTCTCTGGAGCCCGAGGATTTTGCCGTGTAC
TATTGTCAGCAGTTTAGCAGTTCACCAAGCACATTCGGGCAGGGCACCAAGCTGGAAATCAAG
GGCGGCGGCGGCTCTGGCGGAGGAGGCAGCGGCGGAGGAGGCTCCCAGGTGCAGCTGGT
CCAGAGTGGGGCAGAAGTCAAGAAACCCGGAGCAAGTGTCAAAGTGTCCTGTAAAGCCTCAC
GATTCACATTTACCGACTATAACATCCACTGGGTGAGACAGGCACCAGGACAGAGGCTGGAGT
GGATCGGCTATATCAACCCTAACAATGGCGACATCTTCTACAAGCAGAAGTTTAATGGCCGGG
TGACCATCACAAGAGATACCAGCGCCTCCACAGCCTATATGGAGCTGAGCTCCCTGCGGAGCG
AGGATACCGCCGTGTACTATTGCGTGAGGTCTAGCAATATCCGCTACACATTCGACCGGTTCTT
TGACGTGTGGGGCCAGGGCACCCTGGTGACAGTGTCC
hCT3-4 (VL-VH) amino acid sequence
(SEQ ID NO: 22)
EIVLTQSPATLSLSPGERATLSCRASSSVNYIYWYQQKPGQAPRLWIYYTSNLAPGIPARFSGSGSGT
DFTLTISSLEPEDFAVYYCQQFSSSPSTFGQGTKLEIKGGGGSGGGGSGGGGS
QVQLVQSGAEVKKPGASVKVSCKASRFTFTDYNIHWVRQAPGQRLEWIGYINPNNGDIFYKQKFN
GRVTITRDTSASTAYMELSSLRSEDTAVYYCVRSSNIRYTFDRFFDVWGQGTLVTVS

### V. GPC2-Targeted CAR Sequences

CT3 scFv, and scFv of humanized versions of CT3 (hCT3-1, hCT3-2, hCT3-3 and hCT3-4), were used to generate CAR constructs that specifically target GPC2-expressing cells. As disclosed herein, GPC2-targeted CAR constructs with a CD28 hinge region and a CD28 transmembrane domain were superior to CAR constructs having a CD8 hinge region paired with either a CD8 transmembrane domain or a CD28 transmembrane domain. Nucleotide and amino acid sequences of the CAR components, as well as the complete amino acid sequence of three specific CAR constructs (CT3.28H.BBz, CT3.8H.BBz and CT3.8H.28BBz and) are provided below.

CD28 hinge region nucleotide sequence
(SEQ ID NO: 23)
ATCGAAGTCATGTATCCCCCCCCCTATCTGGACAACGAAAAGAGTAACGGAACTATCATTCAC
GTCAAAGGAAAACACCTGTGCCCTAGCCCACTGTTCCCCGGCCCTTCCAAGCCC
CD28 hinge region amino acid sequence
(SEQ ID NO: 24)
IEVMYPPPYLDNEKSNGTIIHVKGKHLCPSPLFPGPSKP
CD28 TM domain nucleotide sequence
(SEQ ID NO: 25)
TTTTGGGTGCTGGTGGTGGTGGGCGGCGTGCTGGCTTGCTATTCCCTGCTGGTCACAGTCGCTTT
TATTATTTTCTGGGTG
CD28 TM domain amino acid sequence
(SEQ ID NO: 26)
FWVLVVVGGVLACYSLLVTVAFIIFWV
4-1BB signaling moiety nucleotide sequence
(SEQ ID NO: 27)
AAACGGGGCAGAAAGAAACTCCTGTATATATTCAAACAACCATTTATGAGACCAGTACAAACT
ACTCAAGAGGAAGATGGCTGTAGCTGCCGATTTCCAGAAGAAGAAGAAGGAGGATGTGAACT
G
4-1BB signaling moiety amino acid sequence
(SEQ ID NO: 28)
KRGRKKLLYIFKQPFMRPVQTTQEEDGCSCRFPEEEEGGCEL
CD3ζ signaling domain nucleotide sequence
(SEQ ID NO: 29)
AGAGTGAAGTTCAGCAGGAGCGCAGACGCCCCCGCGTACCAGCAGGGCCAGAACCAGCTCTAT
AACGAGCTCAATCTAGGACGAAGAGAGGAGTACGATGTTTTGGACAAGAGACGTGGCCGGGA
CCCTGAGATGGGGGGAAAGCCGAGAAGGAAGAACCCTCAGGAAGGCCTGTACAATGAACTGC
AGAAAGATAAGATGGCGGAGGCCTACAGTGAGATTGGGATGAAAGGCGAGCGCCGGAGGGGC
AAGGGGCACGATGGCCTTTACCAGGGTCTCAGTACAGCCACCAAGGACACCTACGACGCCCTT
CACATGCAGGCCCTGCCCCCTCGC
CD3ζ signaling domain amino acid sequence
(SEQ ID NO: 30)
RVKFSRSADAPAYQQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR
GMCSFR signal sequence nucleotide sequence
(SEQ ID NO: 31)
ATGCTTCTCCTGGTGACAAGCCTTCTGCTCTGTGAGTTACCACACCCAGCATTCCTCCTGATCCC
A
GMCSFR signal sequence amino acid sequence
(SEQ ID NO: 32)
MLLLVTSLLLCELPHPAFLLIP
T2A self-cleaving peptide nucleotide sequence
(SEQ ID NO: 33)
GAGGGCAGAGGAAGTCTTCTAACATGCGGTGACGTGGAGGAGAATCCCGGCCCT
T2A self-cleaving peptide amino acid sequence
(SEQ ID NO: 34)
EGRGSLLTCGDVEENPGP
hEGFRt nucleotide sequence
(SEQ ID NO: 35)
CGCAAAGTGTGTAACGGAATAGGTATTGGTGAATTTAAAGACTCACTCTCCATAAATGCTACG
AATATTAAACACTTCAAAAACTGCACCTCCATCAGTGGCGATCTCCACATCCTGCCGGTGGCAT
TTAGGGGTGACTCCTTCACACATACTCCTCCTCTGGATCCACAGGAACTGGATATTCTGAAAAC
CGTAAAGGAAATCACAGGGTTTTTGCTGATTCAGGCTTGGCCTGAAAACAGGACGGACCTCCA
TGCCTTTGAGAACCTAGAAATCATACGCGGCAGGACCAAGCAACATGGTCAGTTTTCTCTTGCA
GTCGTCAGCCTGAACATAACATCCTTGGGATTACGCTCCCTCAAGGAGATAAGTGATGGAGAT
GTGATAATTTCAGGAAACAAAAATTTGTGCTATGCAAATACAATAAACTGGAAAAAACTGTTT
GGGACCTCCGGTCAGAAAACCAAAATTATAAGCAACAGAGGTGAAAACAGCTGCAAGGCCAC
AGGCCAGGTCTGCCATGCCTTGTGCTCCCCCGAGGGCTGCTGGGGCCCGGAGCCCAGGGACTG
CGTCTCTTGCCGGAATGTCAGCCGAGGCAGGGAATGCGTGGACAAGTGCAACCTTCTGGAGGG
TGAGCCAAGGGAGTTTGTGGAGAACTCTGAGTGCATACAGTGCCACCCAGAGTGCCTGCCTCA
GGCCATGAACATCACCTGCACAGGACGGGGACCAGACAACTGTATCCAGTGTGCCCACTACAT
TGACGGCCCCCACTGCGTCAAGACCTGCCCGGCAGGAGTCATGGGAGAAAACAACACCCTGGT
CTGGAAGTACGCAGACGCCGGCCATGTGTGCCACCTGTGCCATCCAAACTGCACCTACGGATG
CACTGGGCCAGGTCTTGAAGGCTGTCCAACGAATGGGCCTAAGATCCCGTCCATCGCCACTGG
GATGGTGGGGGCCCTCCTCTTGCTGCTGGTGGTGGCCCTGGGGATCGGCCTCTTCATG
hEGFRt amino acid sequence
(SEQ ID NO: 36)
RKVCNGIGIGEFKDSLSINATNIKHFKNCTSISGDLHILPVAFRGDSFTHTPPLDPQELDILKTVKEITG
FLLIQAWPENRTDLHAFENLEIIRGRTKQHGQFSLAVVSLNITSLGLRSLKEISDGDVIISGNKNLCYA
NTINWKKLFGTSGQKTKIISNRGENSCKATGQVCHALCSPEGCWGPEPRDCVSCRNVSRGRECVDK
CNLLEGEPREFVENSECIQCHPECLPQAMNITCTGRGPDNCIQCAHYIDGPHCVKTCPAGVMGENN
TLVWKYADAGHVCHLCHPNCTYGCTGPGLEGCPTNGPKIPSIATGMVGALLLLLVVALGIGLFM
CT3.28H.BBz nucleotide sequence
(CT3-CD28 hinge-CD28 TM-4-1BB-CD3ζ is underlined; nucleotides 73-1470)
(SEQ ID NO: 37)
ATGCTTCTCCTGGTGACAAGCCTTCTGCTCTGTGAGTTACCACACCCAGCATTCCTCCTGATCCC
ACATATGGAGGTCCAGCTGCAACAGTCTGGACCTGAACTGGTGAAGCCTGGGGCTTCAGTAAA
GATGTCCTGCAAGGCTTCTAGATTCACATTCACTGACTACAACATACACTGGGTGAAGCAGAG
CCCTGGAAAGACCCTTGAATGGATTGGATATATTAACCCTAACAATGGTGATATTTTCTACAAA
CAGAAGTTCAATGGCAAGGCCACATTGACTATAAACAAGTCCTCCAACACAGCCTACATGGAG
CTCCGCAGCCTGACATCGGAGGATTCTGCAGTCTATTACTGTGTAAGATCCTCTAATATTCGTT
ATACTTTCGACAGGTTCTTCGATGTCTGGGGCACAGGGACCACGGTCACCGTCTCCTCAGGCGG
AGGCGGATCAGGTGGTGGCGGATCTGGAGGTGGCGGAAGCGAAAATGTGCTCACCCAGTCTCC
AGCAATCATGTCTGCATCTCTAGGGGAGAAGGTCACCATGAGCTGCAGGGCCAGCTCAAGTGT
AAATTACATTTACTGGTACCAGCAGAAGTCAGATGCCTCCCCCAAACTATGGATTTATTACACA
TCCAACCTGGCTCCTGGAGTCCCAGCTCGCTTCAGTGGCAGTGGGTCTGGGAACTCTTATTCTC
TCACAATCAGCAGCATGGAGGGTGAAGATGCTGCCACTTATTACTGCCAGCAGTTTTCTAGTTC
CCCATCCACGTTCGGTACTGGGACCAAGCTGGAGCTGAAAACTAGTATCGAAGTCATGTATCC
CCCCCCCTATCTGGACAACGAAAAGAGTAACGGAACTATCATTCACGTCAAAGGAAAACACCT
GTGCCCTAGCCCACTGTTCCCCGGCCCTTCCAAGCCCTTTTGGGTGCTGGTGGTGGTGGGCGGC
GTGCTGGCTTGCTATTCCCTGCTGGTCACAGTCGCTTTTATTATTTTCTGGGTGAAACGGGGCAG
AAAGAAACTCCTGTATATATTCAAACAACCATTTATGAGACCAGTACAAACTACTCAAGAGGA
AGATGGCTGTAGCTGCCGATTTCCAGAAGAAGAAGAAGGAGGATGTGAACTGAGAGTGAAGT
TCAGCAGGAGCGCAGACGCCCCCGCGTACCAGCAGGGCCAGAACCAGCTCTATAACGAGCTCA
ATCTAGGACGAAGAGAGGAGTACGATGTTTTGGACAAGAGACGTGGCCGGGACCCTGAGATG
GGGGGAAAGCCGAGAAGGAAGAACCCTCAGGAAGGCCTGTACAATGAACTGCAGAAAGATAA
GATGGCGGAGGCCTACAGTGAGATTGGGATGAAAGGCGAGCGCCGGAGGGGCAAGGGGCACG
ATGGCCTTTACCAGGGTCTCAGTACAGCCACCAAGGACACCTACGACGCCCTTCACATGCAGG
CCCTGCCCCCTCGCGAGGGCAGAGGAAGTCTTCTAACATGCGGTGACGTGGAGGAGAATCCCG
GCCCTATGCTTCTCCTGGTGACAAGCCTTCTGCTCTGTGAGTTACCACACCCAGCATTCCTCCTG
ATCCCACGCAAAGTGTGTAACGGAATAGGTATTGGTGAATTTAAAGACTCACTCTCCATAAAT
GCTACGAATATTAAACACTTCAAAAACTGCACCTCCATCAGTGGCGATCTCCACATCCTGCCGG
TGGCATTTAGGGGTGACTCCTTCACACATACTCCTCCTCTGGATCCACAGGAACTGGATATTCT
GAAAACCGTAAAGGAAATCACAGGGTTTTTGCTGATTCAGGCTTGGCCTGAAAACAGGACGGA
CCTCCATGCCTTTGAGAACCTAGAAATCATACGCGGCAGGACCAAGCAACATGGTCAGTTTTCT
CTTGCAGTCGTCAGCCTGAACATAACATCCTTGGGATTACGCTCCCTCAAGGAGATAAGTGATG
GAGATGTGATAATTTCAGGAAACAAAAATTTGTGCTATGCAAATACAATAAACTGGAAAAAAC
TGTTTGGGACCTCCGGTCAGAAAACCAAAATTATAAGCAACAGAGGTGAAAACAGCTGCAAGG
CCACAGGCCAGGTCTGCCATGCCTTGTGCTCCCCCGAGGGCTGCTGGGGCCCGGAGCCCAGGG
ACTGCGTCTCTTGCCGGAATGTCAGCCGAGGCAGGGAATGCGTGGACAAGTGCAACCTTCTGG
AGGGTGAGCCAAGGGAGTTTGTGGAGAACTCTGAGTGCATACAGTGCCACCCAGAGTGCCTGC
CTCAGGCCATGAACATCACCTGCACAGGACGGGGACCAGACAACTGTATCCAGTGTGCCCACT
ACATTGACGGCCCCCACTGCGTCAAGACCTGCCCGGCAGGAGTCATGGGAGAAAACAACACCC
TGGTCTGGAAGTACGCAGACGCCGGCCATGTGTGCCACCTGTGCCATCCAAACTGCACCTACG
GATGCACTGGGCCAGGTCTTGAAGGCTGTCCAACGAATGGGCCTAAGATCCCGTCCATCGCCA
CTGGGATGGTGGGGGCCCTCCTCTTGCTGCTGGTGGTGGCCCTGGGGATCGGCCTCTTCATGTG
A
CT3.28H.BBz amino acid sequence
(SEQ ID NO: 38)
MLLLVTSLLLCELPHPAFLLIPHMEVQLQQSGPELVKPGASVKMSCKASRFTFTDYNIHWVKQSPG
KTLEWIGYINPNNGDIFYKQKFNGKATLTINKSSNTAYMELRSLTSEDSAVYYCVRSSNIRYTFDRF
FDVWGTGTTVTVSSGGGGSGGGGSGGGGSENVLTQSPAIMSASLGEKVTMSCRASSSVNYIYWYQ
QKSDASPKLWIYYTSNLAPGVPARFSGSGSGNSYSLTISSMEGEDAATYYCQQFSSSPSTFGTGTKL
ELKTSIEVMYPPPYLDNEKSNGTIIHVKGKHLCPSPLFPGPSKPFWVLVVVGGVLACYSLLVTVAFII
FWVKRGRKKLLYIFKQPFMRPVQTTQEEDGCSCRFPEEEEGGCELRVKFSRSADAPAYQQGQNQL
YNELNLGRREEYDVLDKRRGRDPEMGGKPRRKNPQEGLYNELQKDKMAEAYSEIGMKGERRRGK
GHDGLYQGLSTATKDTYDALHMQALPPREGRGSLLTCGDVEENPGPMLLLVTSLLLCELPHPAFLL
IPRKVCNGIGIGEFKDSLSINATNIKHFKNCTSISGDLHILPVAFRGDSFTHTPPLDPQELDILKTVKEIT
GFLLIQAWPENRTDLHAFENLEIIRGRTKQHGQFSLAVVSLNITSLGLRSLKEISDGDVIISGNKNLCY
ANTINWKKLFGTSGQKTKIISNRGENSCKATGQVCHALCSPEGCWGPEPRDCVSCRNVSRGRECVD
KCNLLEGEPREFVENSECIQCHPECLPQAMNITCTGRGPDNCIQCAHYIDGPHCVKTCPAGVMGEN
NTLVWKYADAGHVCHLCHPNCTYGCTGPGLEGCPTNGPKIPSIATGMVGALLLLLVVALGIGLFM
CT3.8H.BBz amino acid sequence
(SEQ ID NO: 39)
MLLLVTSLLLCELPHPAFLLIPHMEVQLQQSGPELVKPGASVKMSCKASRFTFTDYNIHWVKQSPG
KTLEWIGYINPNNGDIFYKQKFNGKATLTINKSSNTAYMELRSLTSEDSAVYYCVRSSNIRYTFDRF
FDVWGTGTTVTVSSGGGGSGGGGSGGGGSENVLTQSPAIMSASLGEKVTMSCRASSSVNYIYWYQ
QKSDASPKLWIYYTSNLAPGVPARFSGSGSGNSYSLTISSMEGEDAATYYCQQFSSSPSTFGTGTKL
ELKTSTTTPAPRPPTPAPTIASQPLSLRPEACRPAAGGAVHTRGLDFACDIYIWAPLAGTCGVLLLSL
VITKRGRKKLLYIFKQPFMRPVQTTQEEDGCSCRFPEEEEGGCELRVKFSRSADAPAYQQGQNQLY
NELNLGRREEYDVLDKRRGRDPEMGGKPRRKNPQEGLYNELQKDKMAEAYSEIGMKGERRRGKG
HDGLYQGLSTATKDTYDALHMQALPPREGRGSLLTCGDVEENPGPMLLLVTSLLLCELPHPAFLLIP
RKVCNGIGIGEFKDSLSINATNIKHFKNCTSISGDLHILPVAFRGDSFTHTPPLDPQELDILKTVKEITG
FLLIQAWPENRTDLHAFENLEIIRGRTKQHGQFSLAVVSLNITSLGLRSLKEISDGDVIISGNKNLCYA
NTINWKKLFGTSGQKTKIISNRGENSCKATGQVCHALCSPEGCWGPEPRDCVSCRNVSRGRECVDK
CNLLEGEPREFVENSECIQCHPECLPQAMNITCTGRGPDNCIQCAHYIDGPHCVKTCPAGVMGENN
TLVWKYADAGHVCHLCHPNCTYGCTGPGLEGCPTNGPKIPSIATGMVGALLLLLVVALGIGLFM
CT3.8H.28BBz amino acid sequence
(SEQ ID NO: 40)
MLLLVTSLLLCELPHPAFLLIPHMEVQLQQSGPELVKPGASVKMSCKASRFTFTDYNIHWVKQSPG
KTLEWIGYINPNNGDIFYKQKFNGKATLTINKSSNTAYMELRSLTSEDSAVYYCVRSSNIRYTFDRF
FDVWGTGTTVTVSSGGGGSGGGGSGGGGSENVLTQSPAIMSASLGEKVTMSCRASSSVNYIYWYQ
QKSDASPKLWIYYTSNLAPGVPARFSGSGSGNSYSLTISSMEGEDAATYYCQQFSSSPSTFGTGTKL
ELKTSTTTPAPRPPTPAPTIASQPLSLRPEACRPAAGGAVHTRGLDFACDFWVLVVVGGVLACYSLL
VTVAFIIFWVRSKRSRLLHSDYMNMTPRRPGPTRKHYQPYAPPRDFAAYRSKRGRKKLLYIFKQPF
MRPVQTTQEEDGCSCRFPEEEEGGCELRVKFSRSADAPAYQQGQNQLYNELNLGRREEYDVLDKR
RGRDPEMGGKPRRKNPQEGLYNELQKDKMAEAYSEIGMKGERRRGKGHDGLYQGLSTATKDTY
DALHMQALPPREGRGSLLTCGDVEENPGPMLLLVTSLLLCELPHPAFLLIPRKVCNGIGIGEFKDSLS
INATNIKHFKNCTSISGDLHILPVAFRGDSFTHTPPLDPQELDILKTVKEITGFLLIQAWPENRTDLHA
FENLEIIRGRTKQHGQFSLAVVSLNITSLGLRSLKEISDGDVIISGNKNLCYANTINWKKLFGTSGQK
TKIISNRGENSCKATGQVCHALCSPEGCWGPEPRDCVSCRNVSRGRECVDKCNLLEGEPREFVENS
ECIQCHPECLPQAMNITCTGRGPDNCIQCAHYIDGPHCVKTCPAGVMGENNTLVWKYADAGHVCH
LCHPNCTYGCTGPGLEGCPTNGPKIPSIATGMVGALLLLLVVALGIGLFM

### VI. Chimeric Antigen Receptors (CARs)

Disclosed herein are GPC2-specific CARs and cells (for example, T cells, NK cells, B cells, macrophages and iPSCs) engineered to express CARs. Generally, CARs include a binding moiety, an extracellular hinge/spacer element, a transmembrane region and an intracellular domain that performs signaling functions (Cartellieri et al., J Biomed Biotechnol 2010:956304, 2010; Dai et al., J Natl Cancer Inst 108(7):djv439, 2016). In many instances, the binding moiety is an antigen binding fragment of a monoclonal antibody, such as a scFv or single-domain antibody. The spacer/hinge region typically includes sequences from IgG subclasses, such as IgG1, IgG4, IgD and CD8 domains. In some aspects herein, the hinge region is derived from human CD28. In specific examples, the amino acid sequence of the hinge region includes (or consists of) SEQ ID NO: 24. The transmembrane (TM) domain can be can derived from a variety of different T cell proteins, such as CD3ζ, CD4, CD8, CD28 or inducible T cell co-stimulator (ICOS). In some aspects herein, the TM domain is derived from human CD28. In specific examples, the amino acid sequence of the TM domain includes (or consists of) SEQ ID NO: 26. Several different endodomains have been used to generate CARs. For example, the endodomain can consist of a signaling chain having an ITAM, such as CD3ζ or FcεRIγ. In some instances, the endodomain further includes the intracellular portion of at least one additional co-stimulatory domain, such as CD28, 4-1BB (CD137, TNFRSF9), OX-40 (CD134), ICOS, CD27, MYD88-CD40, killer cell immunoglobulin-like receptor 2DS2 (KIR2DS2) and/or DAP10.

The CAR can also include a signal peptide sequence, e.g., N-terminal to the antigen binding domain. The signal peptide sequence can be any suitable signal peptide sequence, such as a signal sequence from granulocyte-macrophage colony-stimulating factor receptor (GMCSFR), immunoglobulin light chain kappa, or IL-2. While the signal peptide sequence may facilitate expression of the CAR on the surface of the cell, the presence of the signal peptide sequence in an expressed CAR is not necessary for the CAR to function. Upon expression of the CAR on the cell surface, the signal peptide sequence may be cleaved off the CAR. Accordingly, in some aspects, the CAR lacks a signal peptide sequence.

In some aspects, the CARs disclosed herein are expressed from a construct (such as from a lentivirus vector) that also expresses a truncated version of human EGFR (hEGFRt; discussed in more detail in section VII below). The CAR and hEGFRt are separated by a self-cleaving peptide sequence (such as T2A) such that upon expression in a transduced cell, the CAR is cleaved from hEGFRt (see WO 2019/094482, which is herein incorporated by reference in its entirety).

In some aspects disclosed herein, the CAR constructs encode the following amino acid sequences, in the N-terminal to C-terminal direction:

GMCSFRss:
(SEQ ID NO: 32)
MLLLVTSLLLCELPHPAFLLIP
NdeI:
HM
Antigen-binding:
GPC2-specific scFv
SpeI:
TS
CD28 Hinge:
(SEQ ID NO: 24)
IEVMYPPPYLDNEKSNGTIIHVKGKHLCPSPLFPGPSKP
CD28 TM:
SEQ ID NO: 26)
FWVLVVVGGVLACYSLLVTVAFIIFWV;
4-1BB:
(SEQ ID NO: 28)
KRGRKKLLYIFKQPFMRPVQTTQEEDGCSCRFPEEEEGGCEL
CD3ζ:
(SEQ ID NO: 30)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR
T2A:
(SEQ ID NO: 34)
EGRGSLLTCGDVEENPGP
GMCSFRss:
(SEQ ID NO: 32)
MLLLVTSLLLCELPHPAFLLIP
hEGFRt:
(SEQ ID NO: 36)
RKVCNGIGIGEFKDSLSINATNIKHFKNCTSISGDLHILPVAFRGDSFT
HTPPLDPQELDILKTVKEITGFLLIQAWPENRTDLHAFENLEIIRGRTK
QHGQFSLAVVSLNITSLGLRSLKEISDGDVIISGNKNLCYANTINWKKL
FGTSGQKTKIISNRGENSCKATGQVCHALCSPEGCWGPEPRDCVSCRNV
SRGRECVDKCNLLEGEPREFVENSECIQCHPECLPQAMNITCTGRGPDN
CIQCAHYIDGPHCVKTCPAGVMGENNTLVWKYADAGHVCHLCHPNCTYG
CTGPGLEGCPTNGPKIPSIATGMVGALLLLLVVALGIGLFM

Immune cells (such as T cells, NK cells, B cells, or macrophages) or iPSCs expressing the CARs disclosed herein can be used to target a specific cell type, such as a tumor cell, for example a GPC2-positive tumor cell. The use of immune cells (such as T cells) expressing CARs is more universal than standard CTL-based immunotherapy because immune cells expressing CARs are HLA unrestricted and can therefore be used for any patient having a tumor that expresses the target antigen.

Accordingly, provided herein are CARs that include a GPC2-specific antibody (or binding fragment thereof). Also provided are isolated nucleic acid molecules and vectors encoding the CARs, and host cells, such as T cells, NK cells, B cells, macrophages or iPSCs, expressing the CARs. Cells expressing CARs comprised of a GPC2-specific monoclonal antibody can be used for the treatment of cancers that express GPC2, such as neuroblastoma, medulloblastoma, retinoblastoma, acute lymphoblastic leukemia, embryonal rhabdomyosarcoma, alveolar rhabdomyosarcoma, Ewing's sarcoma, desmoplastic small round cell tumor, glioma or osteosarcoma.

### VII. Truncated Human EGFR (hEGFRt)

The human epidermal growth factor receptor is comprised of four extracellular domains, a transmembrane domain and three intracellular domains. The EGFR domains are found in the following N-terminal to C-terminal order: Domain I-Domain II-Domain III-Domain IV-transmembrane (TM) domain-juxtamembrane domain-tyrosine kinase domain-C-terminal tail. Domain I and Domain III are leucine-rich domains that participate in ligand binding. Domain II and Domain IV are cysteine-rich domains and do not make contact with EGFR ligands. Domain II mediates formation of homo- or hetero-dimers with analogous domains from other EGFR family members, and Domain IV can form disulfide bonds with Domain II. The EGFR™ domain makes a single pass through the cell membrane and may play a role in protein dimerization. The intracellular domain includes the juxtamembrane domain, tyrosine kinase domain and C-terminal tail, which mediate EGFR signal transduction (Wee and Wang, Cancers 9(52), doi:10.3390/cancers9050052; Ferguson, Annu Rev Biophys 37:353-373, 2008; Wang et al., Blood 118(5):1255-1263, 2011).

A truncated version of human EGFR, referred to herein as “hEGFRt” includes only Domain III, Domain IV and the TM domain. Thus, hEGFRt lacks Domain I, Domain II, and all three intracellular domains. hEGFRt is not capable of binding EGF and lacks signaling activity. However, this molecule retains the capacity to bind particular EGFR-specific monoclonal antibodies, such as FDA-approved cetuximab (PCT Publication No. WO 2011/056894).

Transduction of immune cells (such as T cells, NK cells, B cells or macrophages) or iPSCs with a construct (such as a lentivirus vector) encoding both hEGFRt and a GPC2-specific CAR disclosed herein allows for selection of transduced cells using labelled EGFR monoclonal antibody cetuximab (ERBITUX™). For example, cetuximab can be labeled with biotin and transduced cells can be selected using anti-biotin magnetic beads, which are commercially available (such as from Miltenyi Biotec). Co-expression of hEGFRt also allows for in vivo tracking of adoptively transferred CAR-expressing cells. Furthermore, binding of cetuximab to cells expressing hEGFRt induces cytotoxicity of ADCC effector cells, thereby providing a mechanism to eliminate transduced immune cells or iPSCs in vivo (Wang et al., Blood 118(5):1255-1263, 2011), such as at the conclusion of therapy.

In some aspects herein, the amino acid sequence of hEGFRt is at least 80%, at least 85%, at least 90%, at least 95%, at least 96%, at least 97%, at least 98% or at least 99% identical to SEQ ID NO: 36. In some examples, the amino acid sequence of hEGFRt comprises or consists of SEQ ID NO: 36. In other aspects, the amino acid sequence of hEGFRt comprises no more than 10, no more than 9, no more than 8, no more than 7, no more than 6, no more than 5, no more than 4, no more than 3, no more than 2 or no more than 1 amino acid substitutions relative to SEQ ID NO: 36. In some examples, the amino acid substitutions are conservative substitutions.

### VIII. CAR-Expressing Cell Compositions and Administration Thereof

Compositions are provided that include CAR-expressing cells in combination with one or more pharmaceutically or physiologically acceptable carriers, diluents or excipients. The CAR-expressing cells can be iPSCs, T cells, such as CD3⁺ T cells, such as CD4⁺ and/or CD8⁺ T cells, NK cells, B cells, macrophages or any other suitable immune cell. Such compositions may include buffers such as neutral buffered saline, phosphate buffered saline and the like; carbohydrates such as glucose, mannose, sucrose, dextrans, or mannitol; proteins; polypeptides or amino acids such as glycine; antioxidants; chelating agents such as EDTA or glutathione; adjuvants (e.g., aluminum hydroxide); and preservatives. In some examples, the cell-containing composition includes a cryopreservative, such as DMSO or glycerol. In some examples the cell-containing composition includes a culture media, such as DMEM or RPMI, and may further include serum, such as FBS. In some examples, the cell-containing composition is frozen or in a liquid form. The cells can be autologous to the recipient. However, the cells can also be heterologous (allogeneic).

With regard to the cells, a variety of aqueous carriers can be used, for example, buffered saline and the like, for introducing the cells. These solutions are sterile and generally free of undesirable matter. These compositions may be sterilized by conventional sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration in these formulations can vary widely, and will be selected primarily based on fluid volumes, viscosities, body weight and the like in accordance with the particular mode of administration selected and the subject's needs.

The precise amount of the composition to be administered can be determined by a physician with consideration of individual differences in age, weight, tumor size/burden, extent of metastasis, and condition of the patient (subject). It can generally be stated that a pharmaceutical composition that includes the CAR-expressing immune cells (T cells, B cells, macrophages and/or NK cells) or iPSCs described herein may be administered at a dosage of 10⁴ to 10⁹ cells/kg body weight, such as 10⁵ to 10⁶ cells/kg body weight, including all integer values within those ranges. Exemplary doses are 10⁶ cells/kg to about 10⁸ cells/kg, such as from about 5×10⁶ cells/kg to about 7.5×10⁷ cells/kg, such as at about 2.5×10⁷ cells/kg, or at about 5.0×10⁷ cells/kg.

A composition can be administered once or multiple times, such as 2, 3, 4, 5, 6, 7, 8, 9 or 10 times at these dosages. The composition can be administered using known immunotherapy infusion techniques (see, e.g., Rosenberg et al., New Eng. J. of Med. 319:1676, 1988). The compositions can be administered daily, weekly, bimonthly or monthly. In some non-limiting examples, the composition is formulated for intravenous administration and is administered multiple times. The quantity and frequency of administration can be determined by such factors as the condition of the subject, and the type and severity of the subject's disease, although appropriate dosages may be determined by clinical trials.

In some aspects, the CAR-encoding nucleic acid molecule is introduced into cells, such as T cells, NK cells, B cells, macrophages or iPSCs, and the subject receives an initial administration of cells, and one or more subsequent administrations of the cells, wherein the one or more subsequent administrations are administered less than 15 days, e.g., 14, 13, 12, 11, 10, 9, 8, 7, 6, 5, 4, 3, or 2 days after the previous administration. In one aspect, more than one administration of the CAR-expressing cells are administered to the subject per week, e.g., 2, 3, or 4 administrations of the CAR-expressing cells of the disclosure are administered per week. In one aspect, the subject receives more than one administration of the CAR-expressing cells per week (e.g., 2, 3 or 4 administrations per week) (also referred to as a cycle), followed by a week of no CAR-expressing cell administrations, and then one or more additional administration of the CAR-expressing cells (e.g., more than one administration of the CAR-expressing cells per week) is administered to the subject. In another aspect, the subject (e.g., a human subject) receives more than one cycle of CAR-expressing cells, and the time between each cycle is less than 10, 9, 8, 7, 6, 5, 4, or 3 days. In one aspect, the CAR-expressing cells are administered every other day for 3 administrations per week. In another aspect, the CAR-expressing cells are administered for at least two, three, four, five, six, seven, eight or more weeks. The dosage of the above treatments to be administered to a patient will vary with the precise nature of the condition being treated and the recipient of the treatment. The scaling of dosages for human administration can be performed according to accepted practices.

In some aspects, CAR-expressing cells can replicate in vivo resulting in long-term persistence that can lead to sustained tumor control. In various aspects, the iPSCs, T cells, macrophages, B cells or NK cells administered to the subject, or the progeny of these cells, persist in the subject for at least four months, five months, six months, seven months. eight months, nine months, ten months, eleven months, twelve months, thirteen months, fourteen months, fifteen months, sixteen months, seventeen months, eighteen months, nineteen months, twenty months, twenty-one months, twenty-two months, twenty-three months, or for years after administration of the cells to the subject. In other aspects, the cells and their progeny are present for less than six months, five months, four months, three months, two months, or one month, e.g., three weeks, two weeks, one week, after administration of the CAR-expressing cells to the subject.

The administration of the disclosed compositions may be carried out in any convenient manner, including by injection, ingestion, transfusion, implantation or transplantation. The disclosed compositions can be administered to a patient trans-arterially, subcutaneously, intradermally, intratumorally, intranodally, intramedullary, intracerebrally, intraventricularly, intracranially, intramuscularly, intra-arterially (including into the hepatic artery (such as HAI) or the femoral artery), by intravenous (i.v.) injection, intraprostatically (e.g., for a prostate cancer), intraosseously, intravitreally, or intraperitoneally. In some aspects, the compositions are administered to a patient by intradermal or subcutaneous injection. In other aspects, the compositions of the disclosure are administered by i.v. injection. In other aspects, the compositions of the disclosure are administered by intra-arterial injection. The compositions can also be injected directly into a tumor or lymph node. In one example, administration is intraosseous, and the cancer treated is a cancer of the bone (e.g., osteosarcoma). In one example, administration is intracerebral, intraventricular, or intracranial and the cancer treated is a cancer of the brain (e.g., neuroblastoma or medulloblastoma). In one example, administration is intravitreal, and the cancer treated is a cancer of the eye (e.g., retinoblastoma).

In some aspects, subjects can undergo leukapheresis, wherein leukocytes are collected, enriched, or depleted ex vivo to select and/or isolate the cells of interest, e.g., T cells, B cells, macrophages and/or NK cells. These cell isolates may be expanded by known methods and treated such that one or more CAR constructs can be introduced, thereby creating an autologous cell that expresses the CAR. In some aspects herein, CAR-expressing cells are generated using lentiviral vectors expressing the CAR and a truncated form of the human EGFR (hEGFRt). Co-expression of hEGFRt allows for selection and purification of CAR-expressing immune cells using an antibody that recognizes hEGFRt (e.g., cetuximab, see PCT Publication No. WO 2011/056894), which is described above in section V.

In some aspects, immune cells (such as T cells, NK cells, B cells and/or macrophages) are isolated from peripheral blood by lysing the red blood cells and in some instances depleting the monocytes, for example, by centrifugation through a PERCOLL™ M gradient or by counterflow centrifugal elutriation. A specific subpopulation of T cells. such as CD3+. CD28+, CD4+, CD8+, CD45RA+, and CD45RO+ T cells, can be further isolated by positive or negative selection techniques. For example, T cells can be isolated by incubation with anti-CD3/anti-CD28 (e.g., 3×28)-conjugated beads, such as DYNABEADS® M-450 CD3/CD28 T, for a time period sufficient for positive selection of the desired T cells, see U.S. Published Application No. US20140271635. In a non-limiting example, the time period is about 30 minutes. In other non-limiting examples, the time period ranges from 30 minutes to 36 hours or longer and all integer values there between. In further non-limiting examples, the time period is at least 1, 2, 3, 4, 5, or 6 hours, 10 to 24 hours, 24 hours or longer. Longer incubation times can be used to isolate T cells in any situation where there are few T cells as compared to other cell types, such as in isolation from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells, subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. Multiple rounds of selection can also be used.

Enrichment of a cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ T cells by negative selection, a monoclonal antibody cocktail typically includes antibodies to CD14, CD20, CD11b, CD16, HLA-DR, and CD8. AT cell population can be selected that expresses one or more cytokines. Methods for screening for cell expression are disclosed in PCT Publication No. WO 2013/126712.

For isolation of a desired population of cells by positive or negative selection, the concentration of cells and surface (e.g., particles such as beads) can be varied to ensure maximum contact of cells and beads. In some aspects, a concentration of 1 billion cells/mi is used. In further aspects, greater than 100 million cells/ml is used. In other aspects, a concentration of cells of 10, 15, 20, 25, 30, 35, 40, 45, 50, 55, 60, 65, 70, 75, 80, 85, 90, 95, or 100 million cells/ml is used. Without being bound by theory, using high concentrations can result in increased cell yield, cell activation, and cell expansion. Lower concentrations of cells can also be used. Without being bound by theory, significantly diluting the mixture of T cells and surface (e.g., particles such as beads), interactions between the particles and cells are minimized. This selects for cells that express high amounts of desired antigens to be bound to the particles. For example, CD4+ T cells express higher levels of CD-28 and are more efficiently captured than CD8+ T cells in dilute concentrations. In some aspects, the concentration of cells used is 5×10⁶/ml. In other aspects, the concentration used can be from about 1×10⁵/ml to 1×10⁶/ml, and any integer value in between.

### IX. Methods of Treatment

Provided herein are methods of treating GPC2-positive cancer in a subject by administering to the subject a therapeutically effective amount of a GPC2-targeted CAR-expressing immune cell (such as T cell, NK cell, B cell or macrophage) of CAR-expressing iPSC as disclosed herein. Also provided herein is a method of inhibiting tumor growth or metastasis in a subject by administering to the subject a therapeutically effective amount of a GPC2-targeted CAR-expressing cell disclosed herein. Thus, in some examples, the methods decrease the size, volume and/or weight of a tumor by at least 10%, at least 20%, at least 30%, at least 50%, at least 50%, at least 75%, at least 90%, at least 95%, at least 98%, at least 99% or 100%, for example relative to the size, volume and/or weight of the tumor prior to treatment. In some examples, the methods decrease the size, volume and/or weight of a metastasis by at least 10%, at least 20%, at least 30%, at least 50%, at least 50%, at least 75%, at least 90%, at least 95%, at least 98%, at least 99% or 100%, for example relative to the size, volume and/or weight of the metastasis prior to treatment. In some examples, the methods increase the survival time of a subject with a GPC2-positive cancer by at least 3 months, at least 6 months, at least 9 months, at least 12 months, at least 18 months, at least 24 months, at last 36 months, at least 48 months, or at least 60 months, for example relative to the survival time in an absence of the treatment provided herein. In some examples, combinations of these effects are achieved.

Specifically provided is a method of treating a GPC2-positive cancer in a subject. In some aspects, the method includes administering to the subject a therapeutically effective amount of an isolated immune cell or iPSC that includes a nucleic acid molecule encoding a GPC2-targeted CAR and a hEGFRt, or administering a therapeutically effective amount of an isolated immune cell or iPSC co-expressing a GPC2-targeted CAR and a hEGFRt. In some aspects, the GPC2-positive cancer is a solid tumor. In specific examples, the GPC2-positive cancer is neuroblastoma, medulloblastoma, retinoblastoma, acute lymphoblastic leukemia, embryonal rhabdomyosarcoma, alveolar rhabdomyosarcoma, Ewing's sarcoma, desmoplastic small round cell tumor, glioma or osteosarcoma. In some aspects, the GPC2-positive cancer is a pediatric cancer.

In some aspects of the methods disclosed herein, the isolated immune cells are T lymphocytes. In some examples, the T lymphocytes are autologous T lymphocytes. In other aspects, the isolated host cells are NK cells, B cells or macrophages.

A therapeutically effective amount of a CAR-expressing immune cell or iPSC can depend upon the severity of the disease, the type of disease, and the general state of the patient's health. A therapeutically effective amount of CAR-expressing cells and compositions thereof is that which provides either subjective relief of a symptom(s) or an objectively identifiable improvement as noted by the clinician or other qualified observer (such as a decrease in tumor volume or metastasis).

Administration of the CAR-expressing cells and compositions disclosed herein can also be accompanied by administration of other anti-cancer agents or therapeutic treatments (such as surgical resection of a tumor). Any suitable anti-cancer agent can be administered in combination with the compositions disclosed herein. Exemplary anti-cancer agents include, but are not limited to, chemotherapeutic agents, such as, for example, mitotic inhibitors, alkylating agents, anti-metabolites, intercalating antibiotics, growth factor inhibitors, cell cycle inhibitors, enzymes, topoisomerase inhibitors, anti-survival agents, biological response modifiers, anti-hormones (e.g., anti-androgens) and anti-angiogenesis agents. Other anti-cancer treatments include radiation therapy and antibodies (e.g., mAbs) that specifically target cancer cells or other cells (e.g., anti-PD-1, anti-CLTA4, anti-EGFR, or anti-VEGF). In one example, a cancer is treated by administering a GPC2-targeted CAR immune cell (such as iPSC, T cell, NK cell, B cell or macrophage) disclosed herein and one or more therapeutic mAbs, such as one or more of a PD-L1 antibody (e.g., durvalumab, KN035, cosibelimab, BMS-936559, BMS935559, MEDI-4736, MPDL-3280A, or MEDI-4737), or CLTA-4 antibody (e.g., ipilimumab or tremelimumab). In one example, a cancer is treated by administering a GPC2-targeted CAR-expressing cell (such as iPSC, T cell, NK cell, B cell or macrophage) disclosed herein and one or more mAbs, for example: 3F8, Abagovomab, Adecatumumab, Afutuzumab, Alacizumab, Alemtuzumab, Altumomab pentetate, Anatumomab mafenatox, Apolizumab, Arcitumomab, Bavituximab, Bectumomab, Belimumab, Besilesomab, Bevacizumab, Bivatuzumab mertansine, Blinatumomab, Brentuximab vedotin, Cantuzumab mertansine, Capromab pendetide, Catumaxomab, CC49, Cetuximab, Citatuzumab bogatox, Cixutumumab, Clivatuzumab tetraxetan, Conatumumab, Dacetuzumab, Detumomab, Ecromeximab, Eculizumab, Edrecolomab, Epratuzumab, Ertumaxomab, Etaracizumab, Farletuzumab, Figitumumab, Galiximab, Gemtuzumab ozogamicin, Girentuximab, Glembatumumab vedotin, Ibritumomab tiuxetan, Igovomab, Imciromab, Intetumumab, Inotuzumab ozogamicin, Ipilimumab, Iratumumab, Labetuzumab, Lexatumumab, Lintuzumab, Lorvotuzumab mertansine, Lucatumumab, Lumiliximab, Mapatumumab, Matuzumab, Mepolizumab, Metelimumab, Milatuzumab, Mitumomab, Morolimumab, Nacolomab tafenatox, Naptumomab estafenatox, Necitumumab, Nimotuzumab, Nofetumomab merpentan, Ofatumumab, Olaratumab, Oportuzumab monatox, Oregovomab, Panitumumab, Pemtumomab, Pertuzumab, Pintumomab, Pritumumab, Ramucirumab, Rilotumumab, Rituximab, Robatumumab, Satumomab pendetide, Sibrotuzumab, Sonepcizumab, Tacatuzumab tetraxetan, Taplitumomab paptox, Tenatumomab, TGN1412, Ticilimumab (tremelimumab), Tigatuzumab, TNX-650, Trastuzumab, Tremelimumab, Tucotuzumab celmoleukin, Veltuzumab, Volociximab, Votumumab, Zalutumumab, or combinations thereof.

In one example, a cancer is treated by administering a GPC2-targeted CAR-expressing cell (such as iPSC, T cell, NK cell, B cell or macrophage) disclosed herein and one or more alkylating agents, such as nitrogen mustards (such as mechlorethamine, cyclophosphamide, melphalan, uracil mustard or chlorambucil), alkyl sulfonates (such as busulfan), nitrosoureas (such as carmustine, lomustine, semustine, streptozocin, or dacarbazine). In one example, a cancer is treated by administering a GPC2-targeted CAR-expressing cell (such as iPSC, T cell, NK cell, B cell or macrophage) disclosed herein and cyclophosphamide.

In one example, a cancer is treated by administering a GPC3-targeted CAR-expressing cell (such as iPSC, T cell, NK cell, B cell or macrophage) disclosed herein and one or more antimetabolites, such as folic acid analogs (such as methotrexate), pyrimidine analogs (such as 5-FU or cytarabine), and purine analogs, such as mercaptopurine or thioguanine.

In one example, a cancer is treated by administering a GPC2-targeted CAR-expressing cell (such as iPSC, T cell, NK cell, B cell or macrophage) disclosed herein and one or more natural products, such as include vinca alkaloids (such as vinblastine, vincristine, or vindesine), epipodophyllotoxins (such as etoposide or teniposide), antibiotics (such as dactinomycin, daunorubicin, doxorubicin, bleomycin, plicamycin, or mitomycin C), and enzymes (such as L-asparaginase).

In one example, a cancer is treated by administering a GPC2-targeted CAR-expressing cell (such as iPSC, T cell, NK cell, B cell or macrophage) disclosed herein and one or more platinum coordination complexes (such as cis-diamine-dichloroplatinum II also known as cisplatin), substituted ureas (such as hydroxyurea), methyl hydrazine derivatives (such as procarbazine), and adrenocrotical suppressants (such as mitotane and aminoglutethimide).

In one example, a cancer is treated by administering a GPC2-targeted CAR-expressing cell (such as iPSC, T cell, NK cell, B cell or macrophage) disclosed herein and one or more hormones or antagonists, such as adrenocorticosteroids (such as prednisone), progestins (such as hydroxyprogesterone caproate, medroxyprogesterone acetate, and magestrol acetate), estrogens (such as diethylstilbestrol and ethinyl estradiol), antiestrogens (such as tamoxifen), and androgens (such as testerone proprionate and fluoxymesterone).

In one example, a cancer is treated by administering a GPC2-targeted CAR-expressing cell (such as iPSC, T cell, NK cell, B cell or macrophage) disclosed herein and one or more chemotherapy drugs, such as Adriamycin, Alkeran, Ara-C, BiCNU, Busulfan, CCNU, Carboplatinum, Cisplatinum, Cytoxan, Daunorubicin, DTIC, 5-FU, Fludarabine, Hydrea, Idarubicin, Ifosfamide, Methotrexate, Mithramycin, Mitomycin, Mitoxantrone, Nitrogen Mustard, Taxol (or other taxanes, such as docetaxel), Velban, Vincristine, VP-16, Gemcitabine (Gemzar), Herceptin, Irinotecan (Camptosar, CPT-11), Leustatin, Navelbine, Rituxan STI-571, Taxotere, Topotecan (Hycamtin), Xeloda (Capecitabine), Zevelin and calcitriol. In one example, a cancer is treated by administering a GPC2-targeted CAR-expressing cell (such as iPSC, T cell, NK cell, B cell or macrophage) disclosed herein, cyclophosphamide and fludarabine. In one example, a cancer is treated by administering a GPC2-targeted CAR-expressing cell (such as iPSC, T cell, NK cell, B cell or macrophage) disclosed herein and one or more immunomodulators, such as AS-101 (Wyeth-Ayerst Labs.), bropirimine (Upjohn), gamma interferon (Genentech), GM-CSF (granulocyte macrophage colony stimulating factor; Genetics Institute), IL-2 (Cetus or Hoffman-LaRoche), human immune globulin (Cutter Biological), IMREG (from Imreg of New Orleans, La.), SK&F 106528, and TNF (tumor necrosis factor).Another treatment that can be used in combination with those provided herein is surgical treatment, for example surgical resection of the cancer or a portion of it. Another example of a treatment is radiotherapy, for example administration of radioactive material or energy (such as external beam therapy) to the tumor site to help eradicate the tumor or shrink it prior to surgical resection.

In a specific example, the method includes treating a neuroblastoma by administering to the subject a therapeutically effective amount of (1) an isolated immune cell or iPSC that includes a nucleic acid molecule encoding a GPC2-targeted CAR and a hEGFRt, or administering a therapeutically effective amount of an isolated immune cell or iPSC co-expressing a GPC2-targeted CAR and a hEGFRt. In some examples, the method further includes administering to the subject a therapeutically effective amount of one or more other chemotherapeutic or biological agents. In some aspects, the one or more other chemotherapeutic or biological agents is one or more of 5-FU, cisplatin, gemcitabine, oxaliplatin, doxorubicin, capecitabine, floxuridine, or mitoxantrone, such as gemcitabine plus oxaliplatin (GEMOS), floxuridine, cisplatin, and oxaliplatin, or 5-FU, oxaliplatin and leucovorin (FOLFOX). In some aspects, the one or more other chemotherapeutic or biological agents is one or more of sorafenib, lenvatinib, regorafenib, cabozantinib and ramucirumab. In some aspects, the one or more other chemotherapeutic or biological agents is an immunotherapy drug, such as pembrolizumab and/or nivolumab. In some aspects, the one or more other chemotherapeutic or biological agents is cyclophosphamide, fludarabine, or both.

### EXAMPLES

The following examples are provided to illustrate particular features of certain aspects of the disclosure, but the scope of the claims should not be limited to those features exemplified.

### Example 1: In Vitro Cell Killing by GPC2-Targeted CAR T Cells

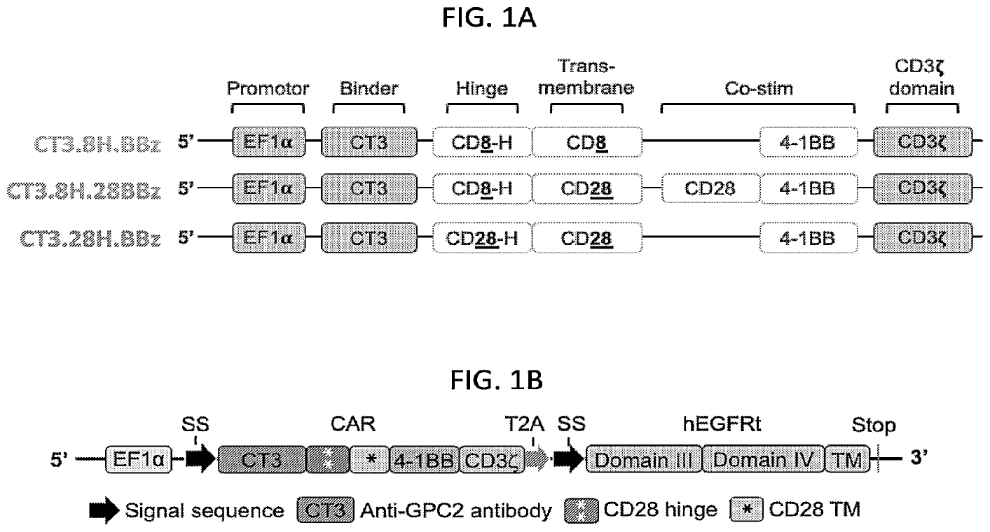

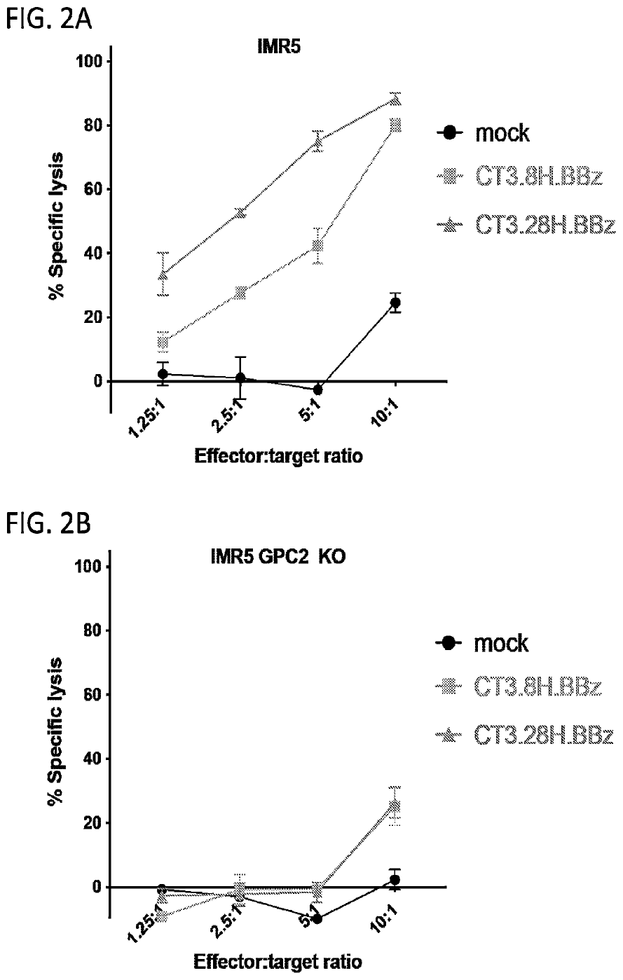

Cell killing mediated by T cells expressing the GPC2-targeted CT3.8H.BBz (also referred to herein as CT3.8H.BBζ) and CT3.28H.BBz (also referred to herein as CT3.28H.BBζ) CARs (see FIGS. 1A-1B) was evaluated using GPC2-positive IMR5 cells and GPC2 knockout (KO) IMR5 cells as the target cells. As shown in FIGS. 2A-2B, CT3.28H.BBz CAR T cells were more potent than CT3.8H.BBz CAR T cells in killing IMR5 cells. Both types of CAR T cells had minimal effect on GPC2 KO-IMR5 cells, demonstrating their GPC2 specificity.

### Example 2: Comparison of CD8 Hinge and CD28 Hinge Containing GPC2-Targeted CARs in an Animal Model of Neuroblastoma Metastasis

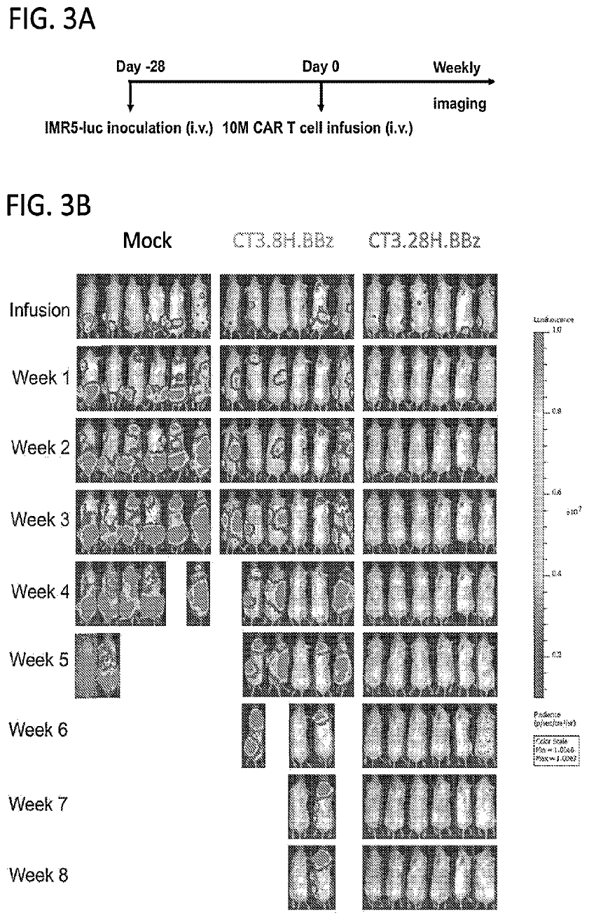

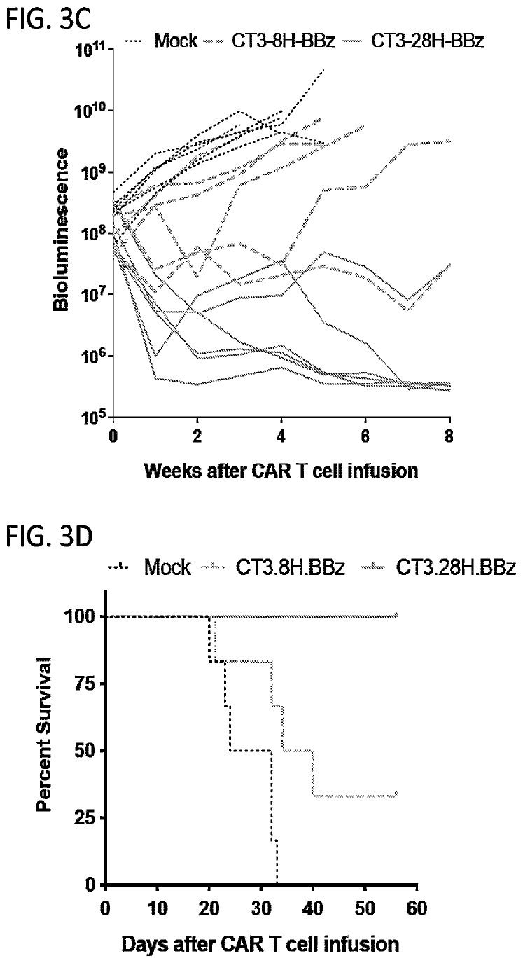

CT3.8H.BBz and CT3.28H.BBz CAR T cells were compared in a mouse model of neuroblastoma (IMR5) metastasis. Mice were i.v. inoculated with IMR5-luc 28 days prior to infusion with 10 million CAR T cells, and were imaged weekly after infusion (FIG. 3A). Bioluminescence images of mock and CAR T cell-treated mice taken weekly for up to eight weeks following T cell infusion are shown in FIG. 3B. Bioluminescence was measured at two, four, six and eight weeks after CAR T cell infusion, the results of which are shown in FIG. 3C. Survival of mock and CAR T cell-treated mice following CAR T cell infusion is shown in FIG. 3D. The results demonstrated that CT3.28H.BBz CAR T cells were significantly more potent than CT3.8H.BBz CAR T cells in regressing neuroblastoma tumors in mice. All mice in the CT3.28H.BBz treatment group survived to the end of this study.

### Example 3: Comparison of CT3.28H.BBz and CT3.8H.28BBz CAR T Cells in an Orthotopic IMR5 Mouse Model

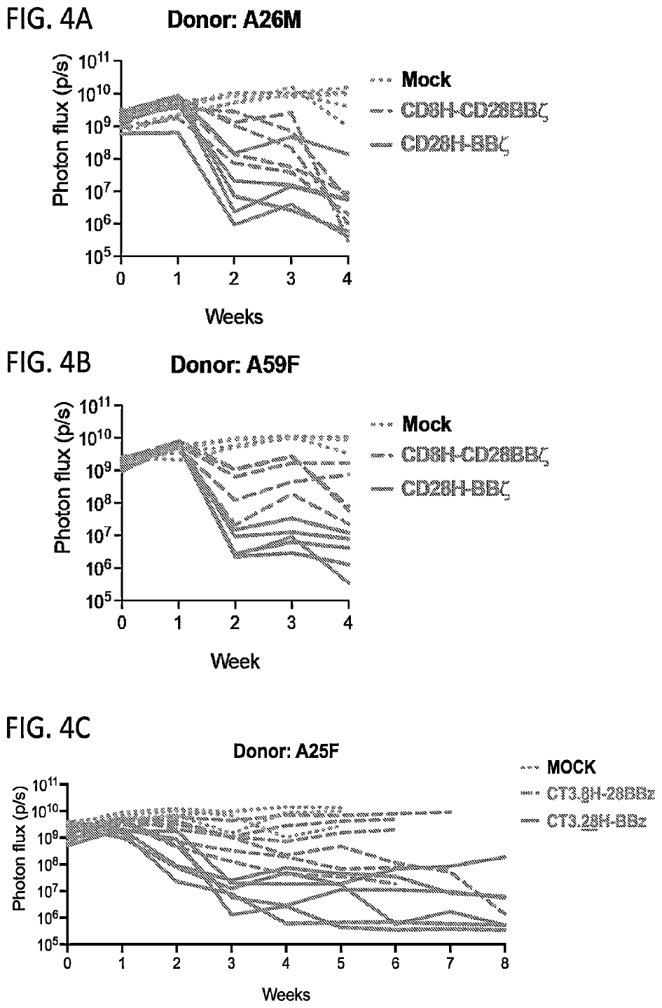

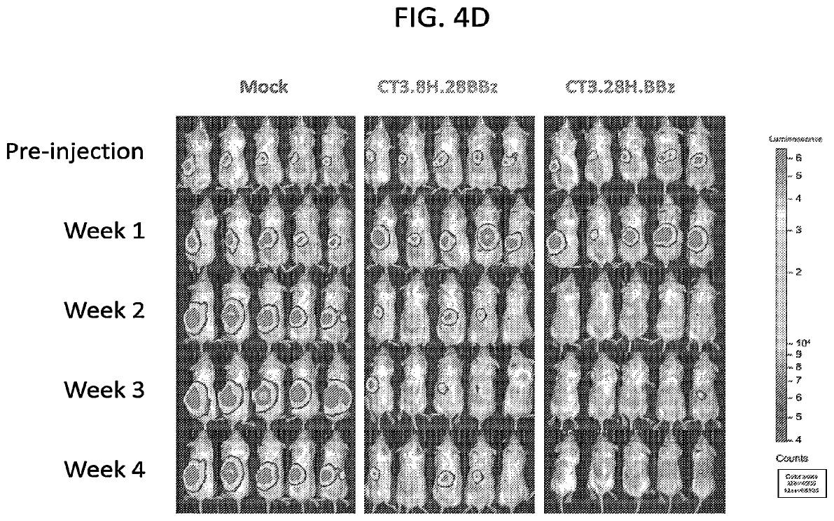

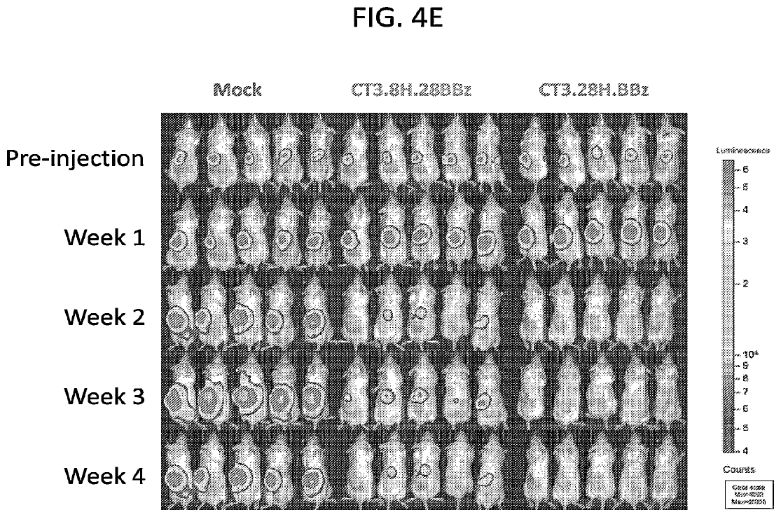

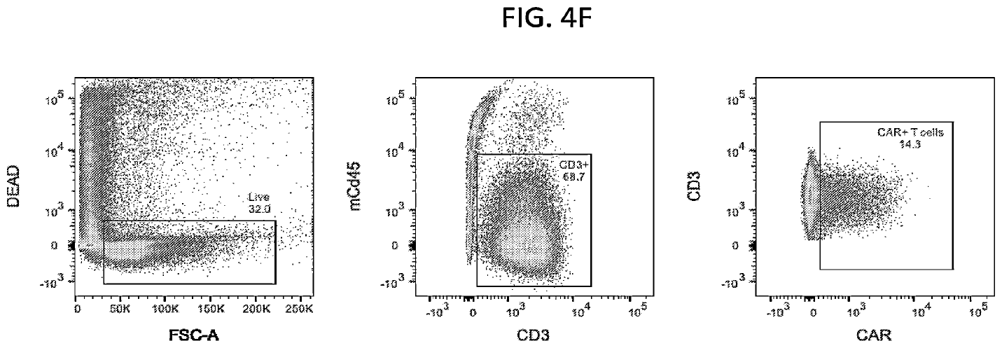

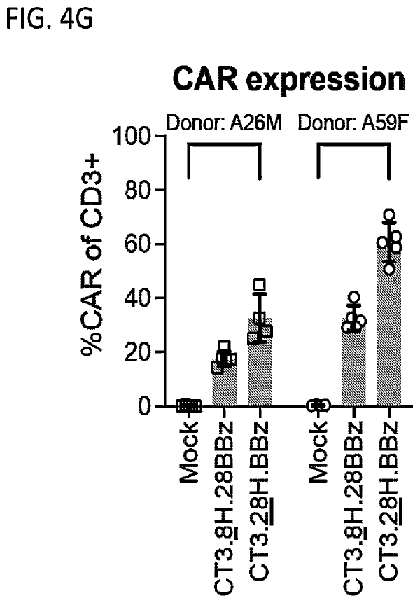

T cells from three different human donors (A26M, A59F and A25F) were used in this study. Mice with a moderate tumor burden were administered 5 million human T cells expressing CT3.28H.BBz CAR or CT3.8H.28BBz CAR and bioluminescence imaging was performed weekly for four (A26M and A59F) or eight (A25F) weeks. As shown in FIGS. 4A-4C, CT3.28H.BBz CAR T cells were superior to CT3.8H.28BBz CAR T cells in reducing tumors for all three T cell donors. Bioluminescence images of mock-treated mice and mice treated with CT3.28H.BBz or CT3.8H.28BBz CAR T cells derived from donors A26M and A59F are shown in FIGS. 4D and 4E, respectively. CT3.28H.BBz CAR T cells were more potent than CT3.8H.28BBz CAR T cells in eradicating moderately sized IMR5 tumors.

Flow cytometry of dissociated spleen samples was performed to assess maintenance of CAR expression in CAR T cell-treated animals. Live cells were gated for CD3+ human cells and the percentage of CAR-positive cells was determined (FIG. 4F). As shown in FIG. 4G, CT3.28H.BBz CAR T cells retained higher levels of CAR expression than CT3.8H.28BBz CAR T cells for both donors tested (A26M and A59F).

### Example 4: CAR Tonic Signaling

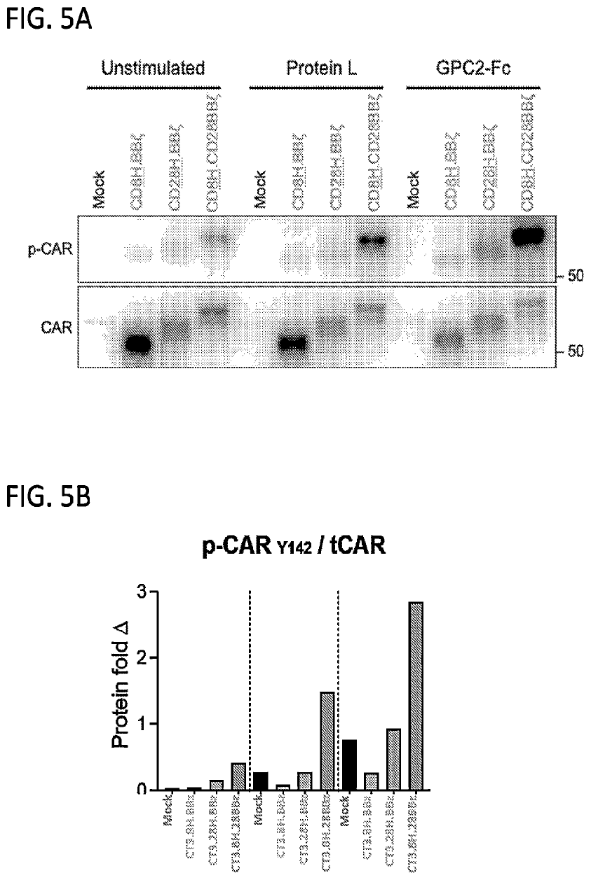

CAR phosphorylation was evaluated as a measure of CAR activation. CT3.8H.BBz, CT3.8H.28BBz and CT3.28H.BBz CAR T cells were unstimulated or stimulated with Protein L or GPC2-Fc and CAR phosphorylation was detected by Western blot (FIG. 5A). The fold change in CAR phosphorylation is shown in FIG. 5B. CT3.8H.28BBz CAR had higher phosphorylation levels than CT3.28H.BBz CAR when tested without stimulation. With GPC2 stimulation, both CARs upregulated their phosphorylation levels. These results demonstrate that CT3.8H.28BBz has more tonic CAR signaling (phosphorylation), which is known to lead to CAR exhaustion. CT3.28H.BBz CAR T cells have lower tonic CAR signaling but show appropriate CAR activation upon antigen presentation (GPC2-Fc).

### Example 5: Comparison of CT3.28H.BBz CAR T Cells and CT3.8H.28BBz CAR T Cells with Low-Dose or High-Dose Chemotherapy in an Orthotopic IMR5 Animal Model

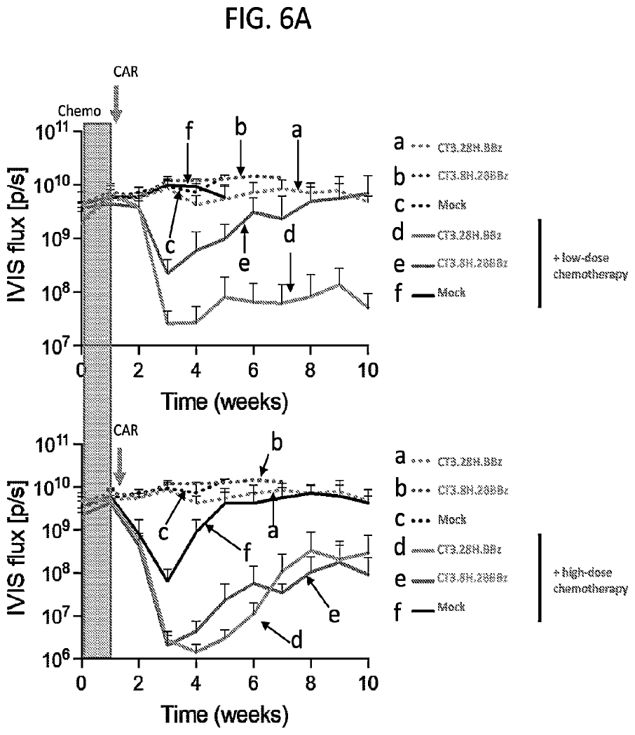

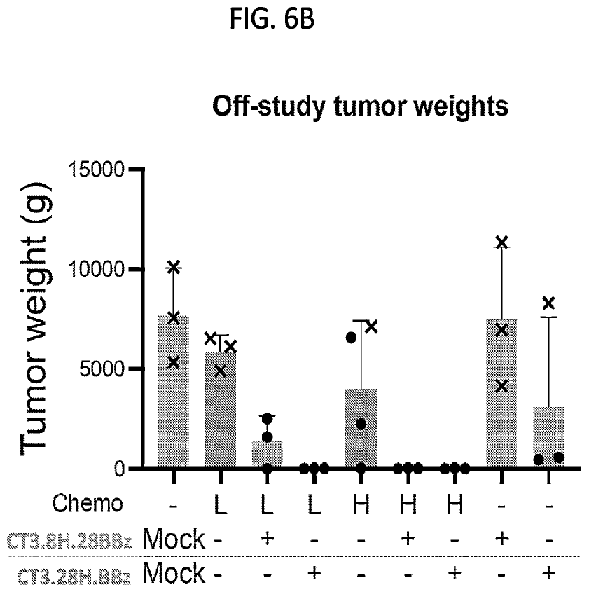

Mice with a large IMR5 tumor burden were treated with no chemotherapy, low-dose chemotherapy, or high-dose chemotherapy (fludarabine/cyclophosphamide) for one week prior to infusion of 5 million CT3.28H.BBz or CT3.8H.28BBz CAR T cells. In this study, T cells were from a single donor. Tumor size as measured by bioluminescence is shown in FIG. 6A. Tumor weight 10 weeks after chemotherapy and CAR T cell infusion is shown in FIG. 6B. These results demonstrate that CT3.28H.BBz outperformed CT3.8H.28BBz when given with conditioning chemotherapy in high-tumor burden mice.

### Example 6: Characterization of Humanized CT3 Antibodies and CARs

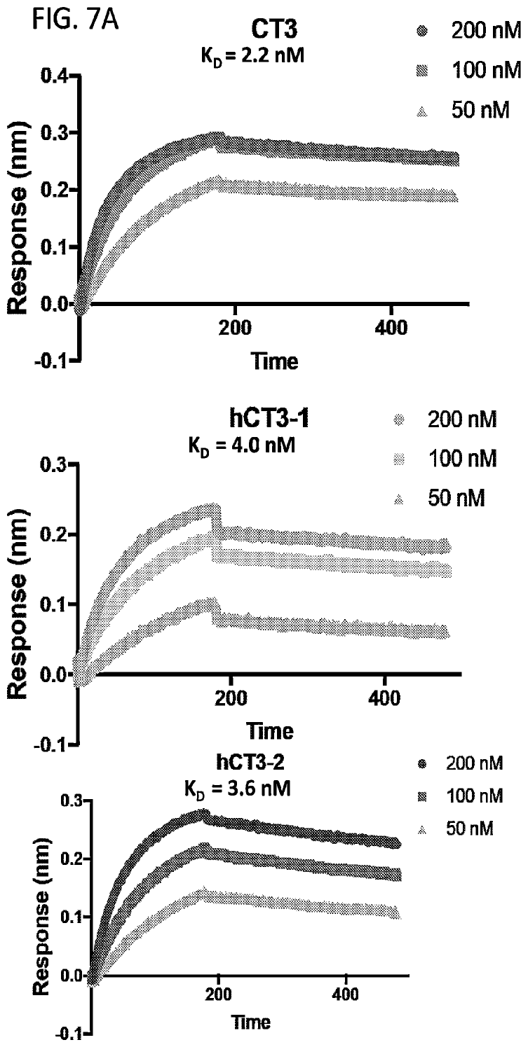

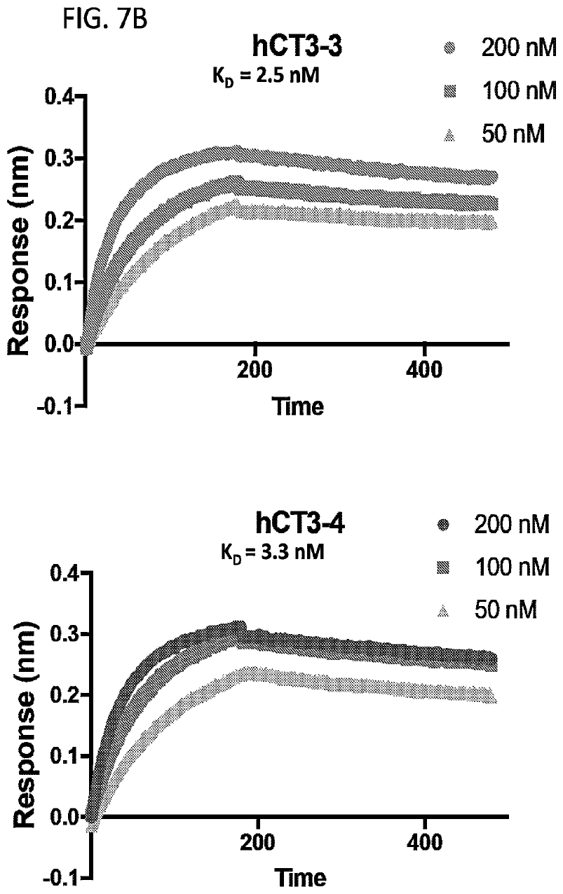

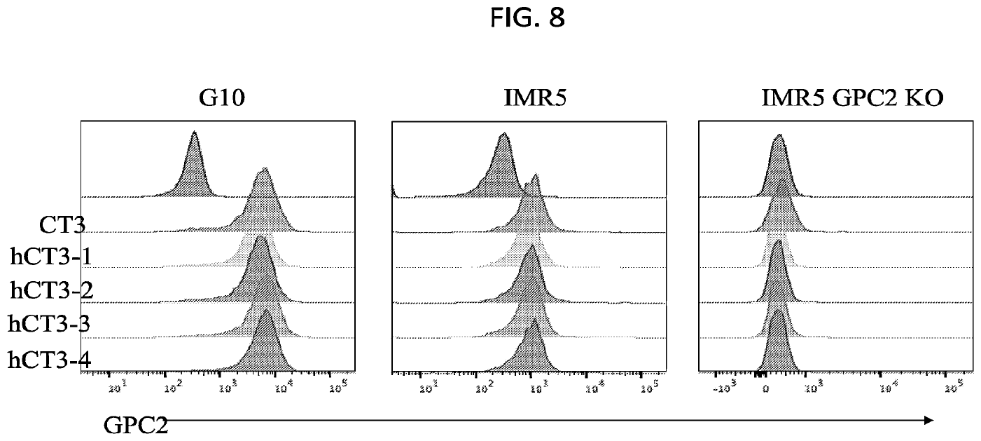

Four humanized forms of murine antibody CT3 were generated: hCT3-1, hCT3-2, hCT3-3 and hCT3-4. Binding affinity of the humanized CT3 antibodies for GPC2 was tested. As shown in FIGS. 7A-7B, the binding affinity of the four humanized CT3 antibodies (4.0 nM, 3.6 nM, 2.5 nM and 3.3 nM) was similar to the binding affinity of the parental CT3 antibody (2.2 nM). In addition, all four humanized antibodies retained the ability to bind GPC2 on the cell surface of G10 and IMR5 cells, but did not exhibit binding to GPC2-KO IMR5 cells (FIG. 8).

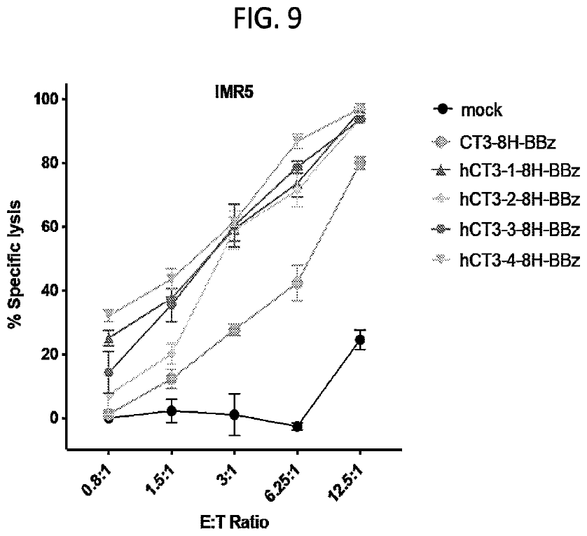

Cell killing by the humanized CT3.8H.BBz CAR T cells was tested. The results demonstrated specific lysis of GPC2-expressing IMR5 cells by CT3-8H-BBz, hCT3-1-8H-BBz, hCT3-2-8H-BBz, hCT3-3-8H-BBz and hCT3-4-8H-BBz CAR T cells (FIG. 9). All four humanized CT3-8H-BBz CAR T cells showed improved killing activity against IMR5 cells as compared to CT3-8H-BBz CAR T cells.

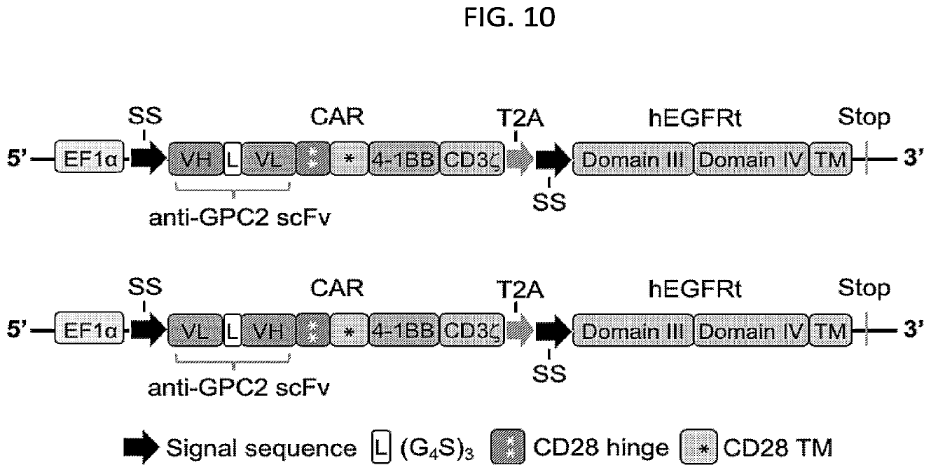

CAR constructs using the humanized CT3 scFvs, in either the VH-linker-VL orientation or the VL-linker-VH orientation, were generated with a CD28 hinge and CD28 transmembrane domain (FIG. 10). It is expected that all eight CAR constructs will potently reduce GPC2-positive tumors and exhibit greater tumor reduction than the corresponding hCT3 CAR constructs having a CD8 hinge.

### Example 7: Materials and Methods

This example describes the materials and experimental procedures used in the studies described in Examples 8-11.

### Cell Lines

The drug-resistant patient-derived xenograft (PDX) SJNBL012407_X1 (MYCN-amplified) was provided by the Children's Solid Tumor Network. IMR-5, CHP-212, SK-N-BE2C, Kelly (also MYCN-amplified) and SHIN, SK-N-FI, SK-N-AS, SH-SHEP, and SH-SY5Y (MYCN-wildtype (WT)) were retrieved from the National Cancer Institute (NCI) Pediatric Oncology Branch cell line repository. NGP-GPC2^{hi}, NBSD-GPC2^{mod}, and SMS-SAN^{lo} (all MYCN-amplified) were provided by Stanford. The GPC2 expression of all lines was determined (Table 3). All cells were confirmed to be mycoplasma free. The cell identity was determined by short-tandem repeat DNA profiling. Stable luciferase (ffLUC)-green fluorescent protein (GFP)-expressing cells were produced by lentiviral transduction and subsequent selection with 0.5 μg/mL of puromycin (Thermo Fisher Scientific). PDX cells were passaged in mice. The NB cell lines were grown in RPMI (Roswell Park Memorial Institute) medium supplemented with 10% fetal bovine serum (FBS) and 100 U/mL penicillin/streptomycin (Gibco).

TABLE 3
GPC2 expression
in neuroblastoma cell lines and xenograft lines

| Cell/xenograft line | MFI | Molecules/cell |
|---|---|---|
| NGP-GPC2^{hi} | 13,483 | 16,177 |
| NBSD-GPC2^{mod} | 6,230 | 7,537 |
| SMS-SAN^{lo} | 3,008 | 3,698 |
| SJNBL012407_X1 | 1,790 | 2,247 |
| IMR-5 | 2,611 | 3,225 |
| SHIN | 1,950 | 2,437 |
| SK-N-FI | 1,197 | 1,540 |
| CHP212 | 745 | 1,002 |
| SK-N-BE2C | 723 | 976 |
| SK-N-AS | 699 | 947 |
| Kelly | 29 | 471 |
| SHEP | 294 | 465 |

### CAR Constructs

As previously described (Li et al., STAR Protoc 2:100942, 2021), the CT3 scFv was cloned into the lentiviral vector, pWPT (Addgene #12255), and different hinge and TM domains (either CD8 or CD28) and co-stimulatory domains (4-1BBζ and/or CD28) were added to generate variations of CAR T-cell constructs. The GD2 CAR sequence was retrieved from publicly available sources (Straathof et al., Sci Transl Med 12:eabd6169, 2020; Pule et al., Nat Med 14:1264-1270, 2008) and cloned into the pWPT vector containing a CD8 or CD28 hinge and TM as well as a 4-1BB co-stimulatory domain. A human truncated extracellular epidermal growth factor receptor domain (hEGFRt) was included as a tag and is recognized by cetuximab.

### Human T Cells and CAR Transduction

Cryopreserved human T cells of healthy volunteer donors (National Institutes of Health Blood Bank) were used for CAR T-cell production as previously described (Li et al., STAR Protoc 2:100942, 2021). Briefly, on Day 0, Lenti-X 293T cells were plated at a density of 2×10⁷ cells per Poly-D-lysine-coated 15 cm dish and subsequently transfected with the CT3 CAR plasmid, envelope plasmid (pMD2.G), and packaging plasmid (psPAX2) at a ratio of 4:1:3 using Lipofectamine 2000 (Thermo Fisher Scientific). The lentivirus-containing supernatants of the Lenti-X 293T cultures were harvested 48-72 hours post-transfection and used to spin-transduce the human T cells. Cryopreserved human T cells were thawed and grown in AIM-V medium (Gibco) supplemented with 10% FBS (Omega Scientific), 100 U/mL penicillin/streptomycin, 1× non-essential amino acids, 0.2 mM L-GlutaMAX, 0.1 mM sodium pyruvate (all Gibco), CD3/CD28-coated Dynabeads (1:1 bead-to-cell ratio, Thermo Fisher Scientific), and 40 IU/mL interleukin (IL)-2 (NCI Frederick BRB Preclinical Repository). The IL-2 concentration was increased to 100 IU/mL after 48 hours at the time of lentiviral transduction. On Day 5 of T-cell culture, the Dynabeads were removed, and the transduced CAR T cells were expanded in culture until Day 8-10 for subsequent downstream assays.

### CAR Western Blot Assay

Manufactured CAR T cells were grown in culture for 3-5 hours while deprived of IL-2. To activate the CAR, either 1.7 μg of GPC2-Fc or 1 μg of Protein L (Acro Biosystems) was added to 2-3×10⁶ cells in a 96-well-round-bottom plate and subsequently cross-linked at 37° C. for varying times. Then, the cells were lysed using radioimmunoprecipitation assay (RIPA) buffer supplemented with Halt protease and phosphatase inhibitor cocktail (Thermo Fisher Scientific). A Bradford Assay (Bio-Rad Laboratories) was used to quantify the protein yields. Samples in the sodium dodecyl sulfate (SDS)-containing buffer were denatured for 10 min. A total of 5-10 μg of protein was resolved by 4-20% SDS-polyacrylamide gel electrophoresis (PAGE) and electroblotted onto a polyvinylidene difluoride membrane. The primary antibodies listed in Table 4 were incubated overnight at 4° C. in 5% bovine serum albumin (BSA) in Tris-buffered saline containing 0.1% Tween-20 (TBST) and 0.02% sodium azide. Secondary antibodies were incubated for 1 hour at room temperature in 5% non-fat dry milk in TBST. Protein bands were visualized using a goat anti-rabbit or anti-mouse IgG-HRP conjugated secondary antibody (200 μg/mL; Santa Cruz Biotechnology) and the SuperSignal West Femto Maximum Sensitivity Substrate (Thermo Fisher Scientific). Enhanced chemiluminescence (Bio-Rad Laboratories) was applied to visualize the bands, which were quantified with ImageJ.

TABLE 4
Antibodies

| Antibody | Clone | Company | Cat. No. |
|---|---|---|---|
| Phospho-CD3 ζ (Y142) | EP265(2)Y | Abcam | ab68235 |
| Anti-CD3-ζ | 6B10.2 | Santa Cruz | sc-1239 |
| Phospho-Zap-70 (Y319)/Syk | 65E4 | CST | 2717 |
| (Y352) | | | |
| Anti-Zap70 | 1E7.2 | Santa Cruz | sc-32760 |
| Phospho-p44/42 MAPK (Erk1/2) | D13.14.4E | CST | 4370 |
| (T202/Y204) | | | |
| Anti-Erk1/2 | 137F5 | CST | 4695 |
| Vinculin | E1E9V | CST | 13901 |

CST: Cell Signaling Technology

### In Vitro Cytotoxicity Assays

CAR T cells and ffLUC-GFP-expressing NB tumor cells were co-cultured at varying effector-to-tumor (E:T) ratios as previously described (Nguyen et al., Cancer Immunol Immunother 67:615-626, 2018). Every 24 hours thereafter, the starting number of tumor cells was added to each well to re-challenge the CAR T cells. The specific lysis of the tumor cells was measured using the ONE-Glo assay. The results were normalized to conditions with untransduced (UT) mock T cells.

### Mice

For all studies, 4-6 weeks old female NOD-SCID (NSG) mice were obtained from the NCI Center for Cancer Research Animal Resource Program.

### Bioluminescence Imaging

IMR-5 with stable expression of ffLUC was used for bioluminescence imaging (BLI). Tumor-bearing mice were injected with d-luciferin potassium salt (150 mg/kg, intraperitoneal (IP)) and imaged on an IVIS Lumina XR System (PerkinElmer) 5 min after d-luciferin injection (1 min acquisition time). Region of interest analysis was performed using the Living Image software (PerkinElmer; V.4.3.1).

### In Vivo Therapy Model

Either PDX cells or IMR-5 were implanted orthotopically (2.5×10⁵) into NSG mice (Li et al., STAR Protoc 2:100942, 2021). Typically, 3 weeks after tumor implantation surgery, animals meeting enrollment criteria with a BLI (>107 photon/s), were randomized to receive either UT mock control T cells or GPC-targeted CAR T cells. The numbers of tail vein injected T cells were based on CAR⁺ T cells. Total T cell numbers in mock groups were adjusted to match those in the CAR groups. For PDX studies, the experiment was terminated on Day 50 after tumor injection. Since BLI signals did not correlate with large tumor burdens, tumor weights were recorded at the end of the study to determine the treatment efficacy. For experiments with IMR-5, tumor-bearing animals were monitored via BLI. The survival of treated mice was monitored until Day 80 (˜11 weeks after tumor implantation). Survival endpoints were death, >20% weight loss from baseline, or severe moribund status as determined by an animal caretaker, who was blinded to the study.

### In Vivo Homing Studies

Mice with IMR-5 WT NB were injected with ffLUC-GFP-expressing GPC2-targeting CAR T cells or UT mock T cells. After tail vein injection of the T cells, the animals were monitored serially via BLI to assess homing and expansion of the T cells in vivo. At the end of the experiments, organs of luciferin-injected mice were removed at 5 min and imaged in 6-well Petri dishes.

### Flow Cytometry

Samples were stained using 1×10⁶ cells. Gates were drawn with fluorescence-minus-one controls. Compensations and voltages were set with single-color controls. The following antibodies were used for the detection of surface epitopes: CD45 (detected by clone HI-30), CD3 (OKT3), CD4 (OKT4), and CD8 (HIT8a). GPC2-Fc and anti-human Fc (M1310G05) or anti-EGFR antibody (AY13) were used to measure CAR transduction efficiency. CT3 antibody and anti-mouse IgG1 (RMG1-1) were used to detect GPC2 expression in tumor cells. The PE Phycoerythrin Fluorescence Quantitation Kit (BD) was used to determine the density of GPC2 expression as per the manufacturer's instructions. Data were collected on a Fortessa LSR machine. Data analysis was conducted with FlowJo V.10.

### Cytokine Bead Assay

Cytokine bead assays (CBAs) were conducted to quantitate the secreted cytokines in the supernatant of T and tumor co-cultures following the manufacturer's instructions (BioLegend).

### Single-Cell RNA-Seq

Two donors were used to manufacture CAR T cells, which we injected into IMR-5-bearing mice on Day 8 of cell manufacturing. The injection cell product was stained with TotalSeq-C antibodies targeting CD8 (cat. #344752) and CD4 (cat. #300567) and subjected to the 10× Genomics 5′ V.3.1 chemistry kit for library generation. Eight days after T cell injection into the mice, tumors were processed into single-cell suspensions with viability>80%. Three tumor samples per treatment group were pooled. About 10,000 cells per group were loaded to capture 6000 cells. The complementary DNA libraries were sequenced on the Illumina NextSeq 2000 and NovaSeq 6000 with a target depth of approximately 50,000 reads per cell.

### Computational Analysis

Single-cell RNA-seq FASTQ files were processed using the CellRanger software suite (V.6.1.2, 10× Genomics) with the corresponding human GRCh38 genome reference. Custom reference (GRCh38+GFP) was used to see if GFP sequences were detected in annotated tumor cells. Cell barcodes were determined based on the distribution of unique molecular identifier (UMI) counts, and a filtered gene-barcode matrix was generated by CellRanger for the downstream analysis in Seurat (V.4.0.1, R package) (Wolock et al., Cell Syst 8:281-291, 2019; Stuart et al., Cell 177:1888-1902, 2019). Cells with low (<200 genes) and greater than 10% of UMIs mapped to mitochondrial genes were removed. Data integration across different samples and treatment groups was performed with reciprocal principal component analysis (Hao et al., Cell 184:3573-3587, 2021) implemented in Seurat. The ‘NormalizeData’ function with parameters: normalization.method=‘LogNormalize’ and scale.factor=10,000 was applied to normalize the expression level of genes in each cell. The ‘FindVariableFeatures’ function with the ‘vst’ method was used to identify 2000 highly variable genes. The ‘ScaleData’ function with default parameters was used to scale and center gene expression matrices. To perform clustering, PCA dimensionality reduction was first conducted with the ‘RunPCA’ function. The first 20 principal components were selected to construct the shared nearest neighbor graph with the ‘FindNeighbors’ function. Clusters were determined using the Louvain algorithm with the ‘FindClusters’ function. SingleR (V.1.8.1, R package; Aran et al., Nat Immunol 20:163-172, 2019) was used to do an initial automatic cell type annotation with the known cell type labels from the Blueprint/ENCODE reference and Database of Immune Cell Expression to predict the identities of cell clusters. Then, it was manually checked whether the annotations were reliable by examining the top-ranked differentially expressed genes of each cluster, which were obtained with the ‘FindAllMarkers’ function with default parameters but with set min.pct=0.25. The uniform manifold approximation and projection (UMAP) was finally applied to visualize the single-cell transcriptional profiles in two-dimensional space. Tumor cells were confirmed by the GFP sequence and copy number variation analysis with infercnv (V.1.10.1, R package; Tickle et al., inferCNV of the Trinity of CTATproject, Cambridge, MA, USA: Klarman Cell Observatory, Broad Institute of MIT and Harvard, 2019). CD45⁺ immune cells were annotated using canonical gene markers. Lymphoid cells were separated from tumor and mouse cells and reclustered to obtain more refined cell clusters. Differential gene expression was calculated for all pairs of clusters and therapy groups. Sample integration across the treatment groups was performed with the standard anchor-based workflow in Seurat. For initial clustering and annotation, k-nearest neighbor (KNN) graph-based clustering was applied on the weighted RNA similarities, to calculate the Jaccard index (neighborhood overlap) between every pair of cells with a high resolution and merging of clusters. UMAP plots were used to visualize the results using the Seurat package. Tumor cells were confirmed by the green fluorescent protein sequence and copy number variation analysis. CD45⁺ immune cells were annotated using canonical gene markers. QIAGEN Ingenuity Pathway Analysis was used for pathway enrichment analysis (QIAGEN) (Kramer et al., Bioinformatics 30:523-530, 2014).

### Statistical Analysis

The Student's t-test (normally distributed data) or Mann-Whitney U test (skewed data) were used to compare two groups and one-way analysis of variance (ANOVA, normally distributed data) followed by Tukey's post hoc comparison tests or a one-way ANOVA on ranks (skewed data) followed by the Dunn test for comparisons with more than two groups. For survival analysis, Kaplan-Meier curves were generated, and a two-sided log-rank test was used to compare survival between the groups. All experiments were performed in biological replicates with at least two donors.

### Example 8: In Vitro Comparison of CT3.28H.BBζ, CT3.8H.BBζ, and CT3.8H.28BBζ

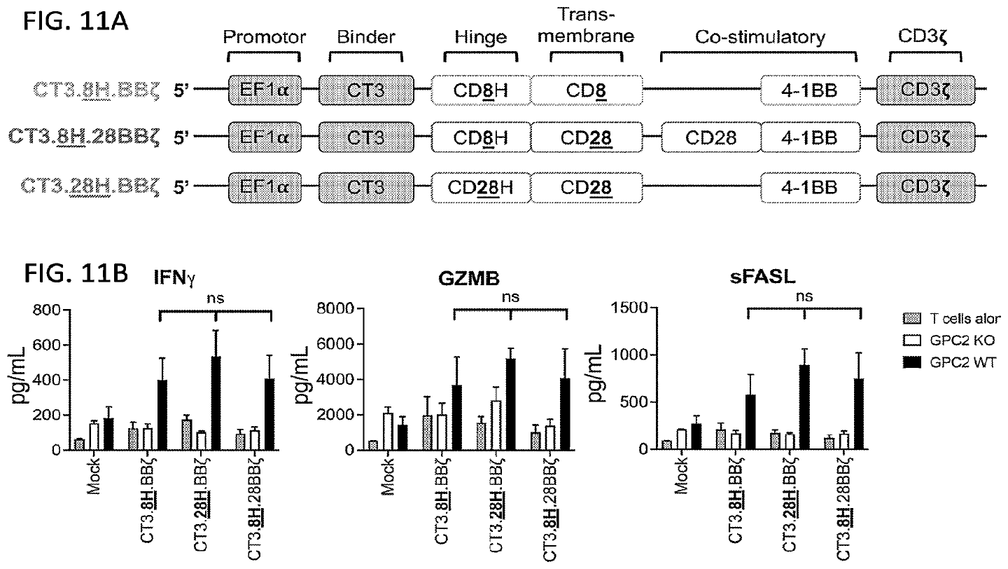

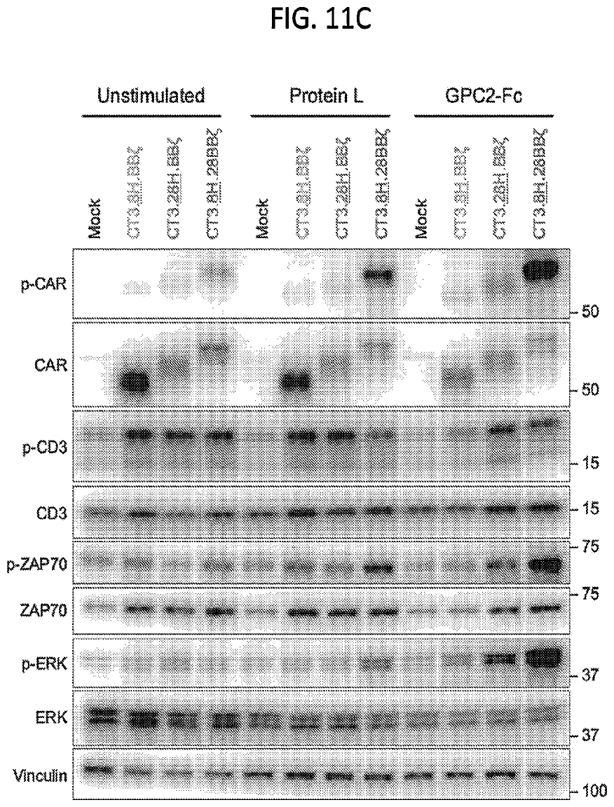

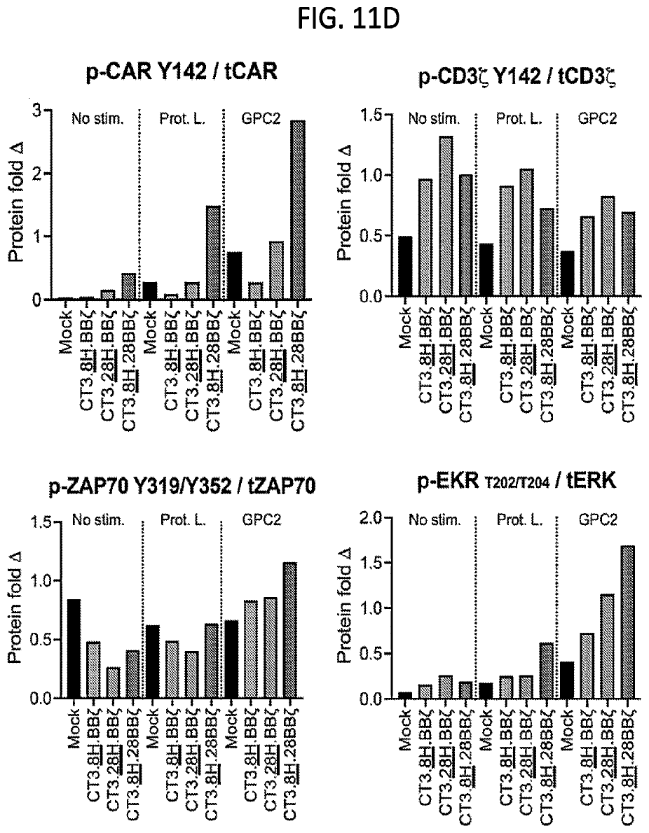

To identify the most efficacious GPC2-CAR construct for clinical translation, a head-to-head comparison was first performed in vitro using CT3 in three different CAR backbone constructs (FIG. 11A): (1) CT3 with a CD8 hinge, CD8 TM, and 4-1BB costimulatory domain (CT3.8H.BBζ, published CAR; Li et al., Cell Rep Med 2:100297, 2021), (2) CT3 with a CD28 hinge, CD28 TM, and 4-1BB costimulatory domain (CT3.28H.BBζ), and (3) CT3 with a CD8 hinge, CD28 TM, and CD28-4-1BB costimulatory domain (CT3.8H.28BBζ). In co-culture with GPC2-WT or GPC2-knock out (KO) IMR-5 tumor cells, all three constructs showed increased levels of interferon-γ, granzyme B (GZMB), and soluble Fas ligand in the supernatant in the presence of the CAR antigen, GPC2 (FIG. 111B). The cytokine levels approximated background levels in conditions containing GPC2-KO cells, indicating specificity of the CARs. However, of the three constructs, CT3.28H.BBζ demonstrated the least tonic signaling indicated by low phosphorylation levels of the CAR and downstream molecules such as ZAP70 or ERK at rest (FIGS. 11C-11D) but an appropriate increase thereof on CAR crosslinking in vitro. Although CT3.8H.BBζ also showed low tonic signaling at rest, the CAR activation was not as robust as with CT3.28H.BBζ after antigen-specific crosslinking. Furthermore, CT3.28H.BBζ functioned better than another GPC2 scFv, GPC2.19 (Heitzeneder et al., Cancer Cell 32:295-309, 2022), in the present CAR backbone, especially at lower E:T ratios and against NB lines with lower antigen densities and better cell expansion and CAR persistence with tumor rechallenge. These data indicate that all three CAR constructs have a comparable function in vitro but that CT3.28H.BBζ lacks tonic signaling, which could positively impact antitumor activity in the context of persistent tumor exposure.

### Example 9: CT3.28H.BBζ Demonstrates Highly Effective Anti-NB Activity In Vivo

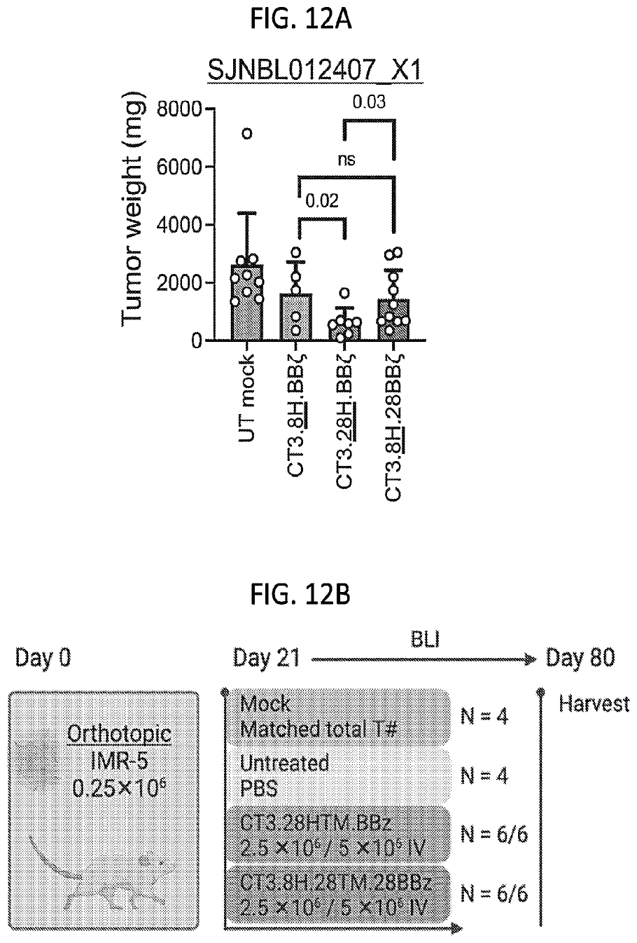

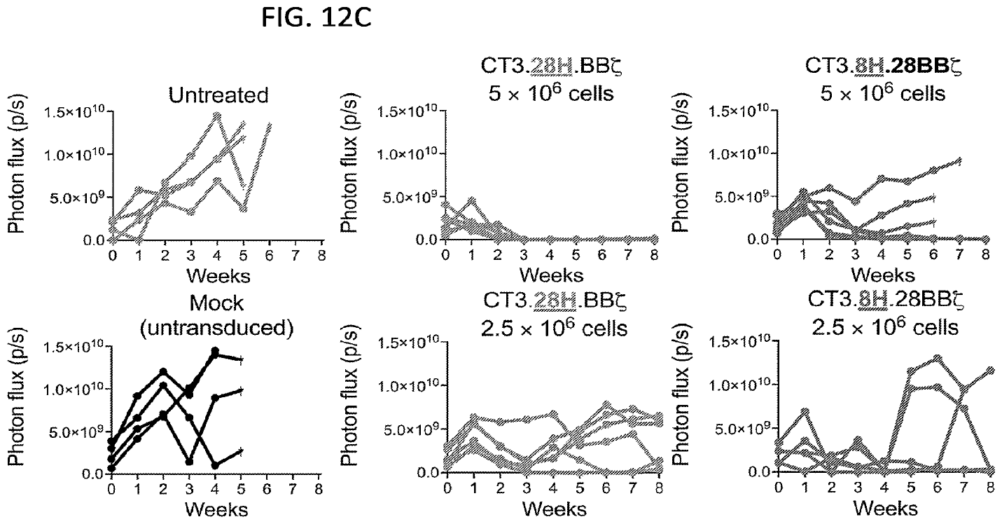

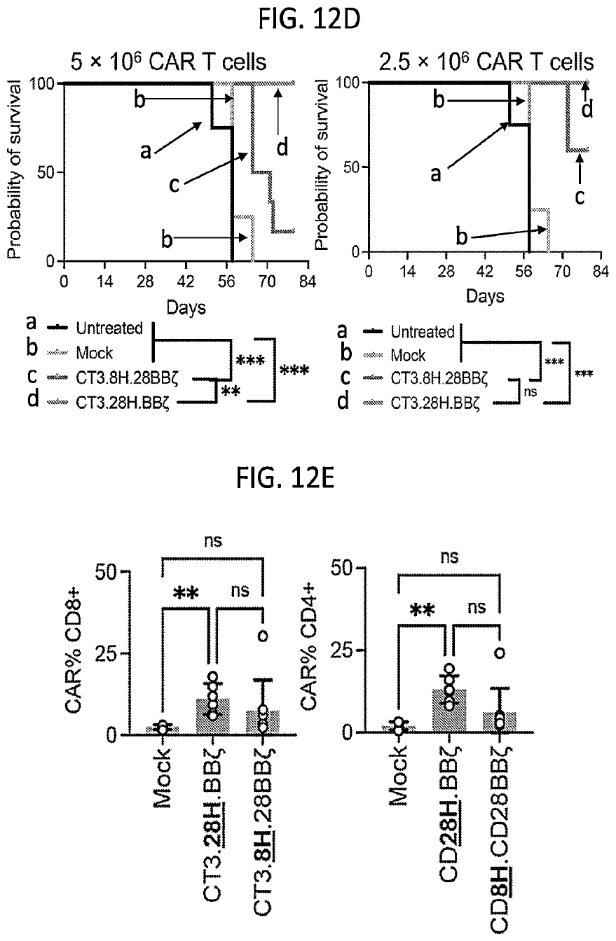

To determine which of the three GPC2-CAR constructs has the best antitumor activity against NB in vivo, an orthotopic PDX model was utilized. The 4-6 week-old NSG mice were injected orthotopically with SJNBL012407_X1. This PDX line has molecular features of high-risk NB (MYCN amplification), and most tumor-bearing mice treated with conventional chemotherapy and/or immunotherapy cannot be cured (Nguyen et al., Neoplasia 26:100776, 2022; Nguyen et al., Clin Cancer Res 28:3785-3796, 2022). Three weeks after tumor implantation, mice were randomized to receive either UT mock T cells or 2.5×10⁶ CAR⁺ T cells. Four weeks post CAR T cell infusion (Day 50 post tumor implantation), CT3.28H.BBζ induced the most significant tumor regression comparing all three CAR constructs (FIG. 12A). Since CT3.8H.BBζ has been previously published (Li et al., Cell Rep Med 2:100297, 2021; Tian et al., J Clin Invest 132:e155621, 2022) and had comparable activity to CT3.8H.28BBζ, subsequent studies focused on CT3.28H.BBζ and CT3.8H.28BBζ. Survival studies were conducted in vivo tumor growth kinetics were evaluated following GPC2-CAR T cell injection. The 4-6 weeks old NSG mice with orthotopic IMR-5.ffLUC-GFP tumors were treated with high (5×10⁶) or low-dose (2.5×10⁶) CAR T cells (FIG. 12B). After high-dose treatment with CT3.28H.BBζ, all animals demonstrated a steep decline in their BLI signal, ultimately approximating background levels (FIG. 12C). Mice treated with high-dose CT3.8H.28BBζ responded temporarily but were unable to maintain tumor control. All mice in both GPC2-CAR T cell groups treated with 2.5×10⁶ CAR T cells demonstrated an initial decline in their BLI but eventually progressed with downregulation of GPC2 in relapsed tumors. The differences in the tumor growth kinetics between mice receiving the high and lower dose of CAR T cells were mirrored in the survival studies of these animals. Tumor-bearing mice treated with high-dose CT3.28H.BBζ CAR T cells exhibited the longest survival (FIG. 12D) and higher levels of tumor-infiltrating CAR⁺ T cells by flow cytometry analysis (FIG. 12E). The survival between the CAR groups was not statistically different at the lower dose level. However, six of six mice never met the study endpoints when treated with CT3.28H.BBζ CAR T cells, while two of five treated with CT3.8H.28BBζ CAR T cells succumbed due to tumor. Although in vitro experiments demonstrated a similar function of the three different GPC2-targeting CARs, the subsequent in vivo studies identified CT3.28H.BBζ as the most potent construct. The superior performance of CT3.28H.BBζ may be due to less tonic signaling, higher levels of CAR⁺ effector cells in the tumor microenvironment (TME), and/or antigen escape. To further evaluate the molecular differences across the three CAR constructs, tumor-infiltrating T cells were analyzed by single-cell RNA-seq.

### Example 10: CT3.28H.BBζ CAR T Cells Upregulate Effector Molecules in the TME

To understand the nature of the T cells expressing the different GPC2-CAR T constructs prior to infusion as well as after encountering tumor in vivo, single-cell RNA-seq was performed on the manufactured GPC2-CAR T as well as on harvested tumor-infiltrating T cells at Day 8, a time prior to tumor regression (typically occurs at Day 10).

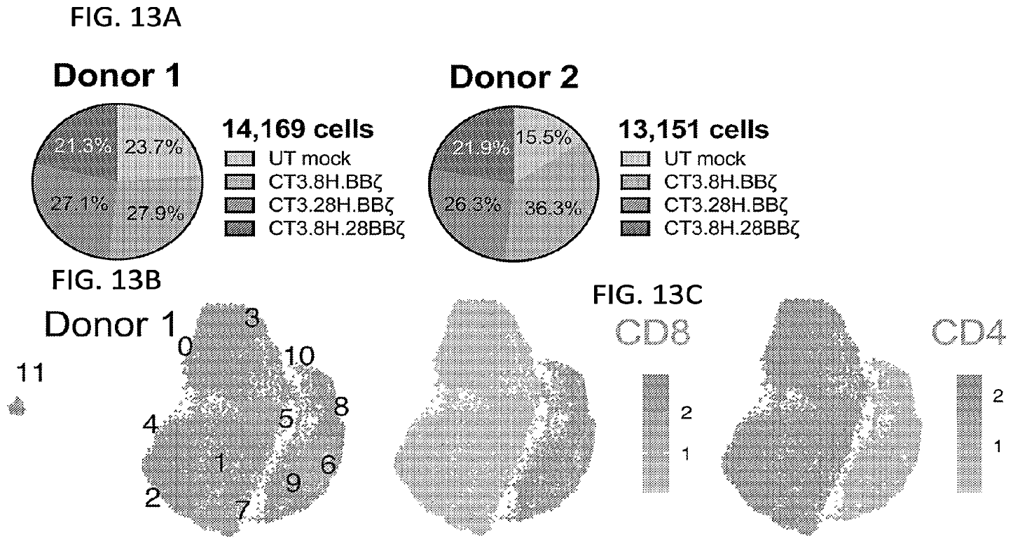

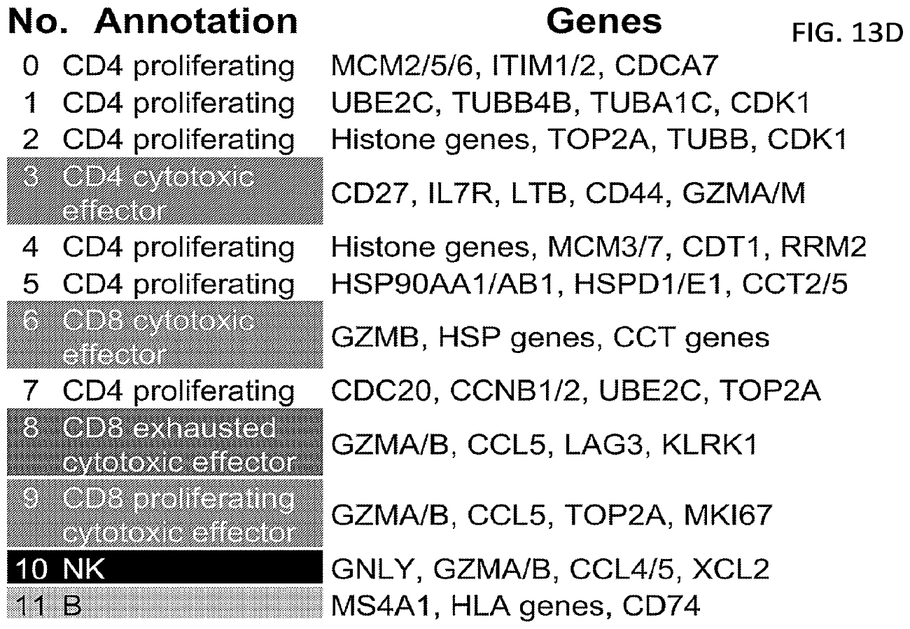

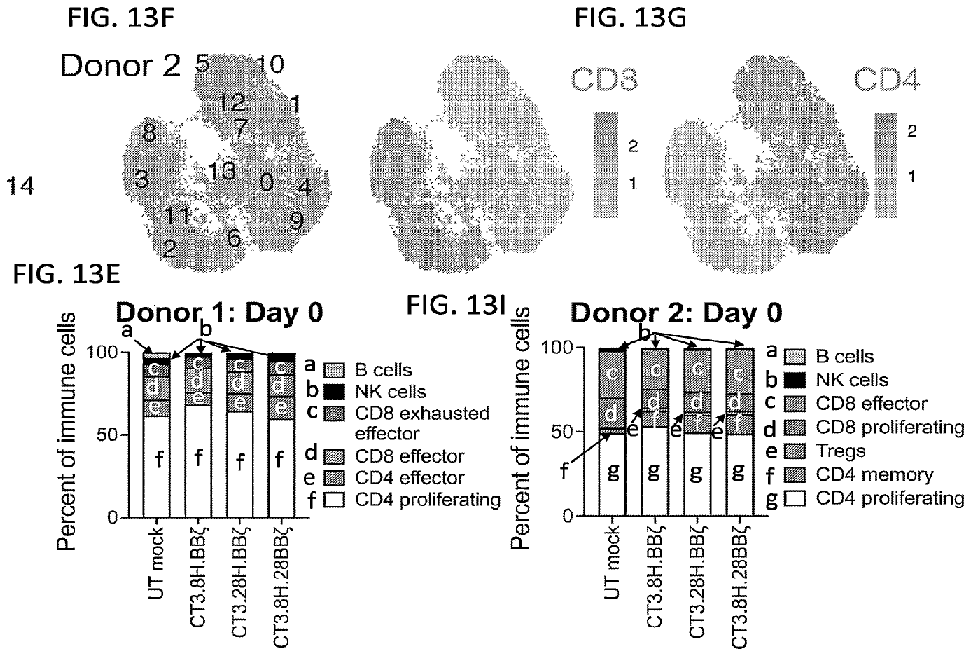

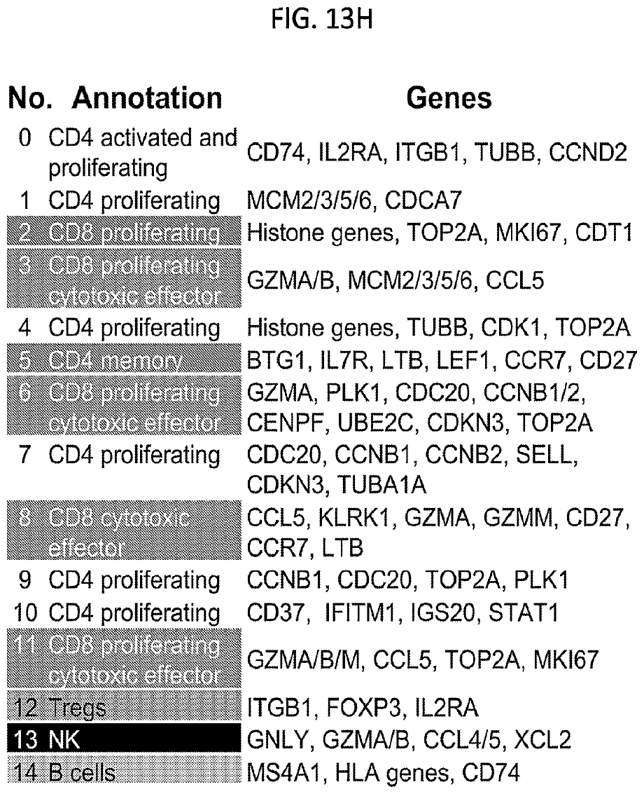

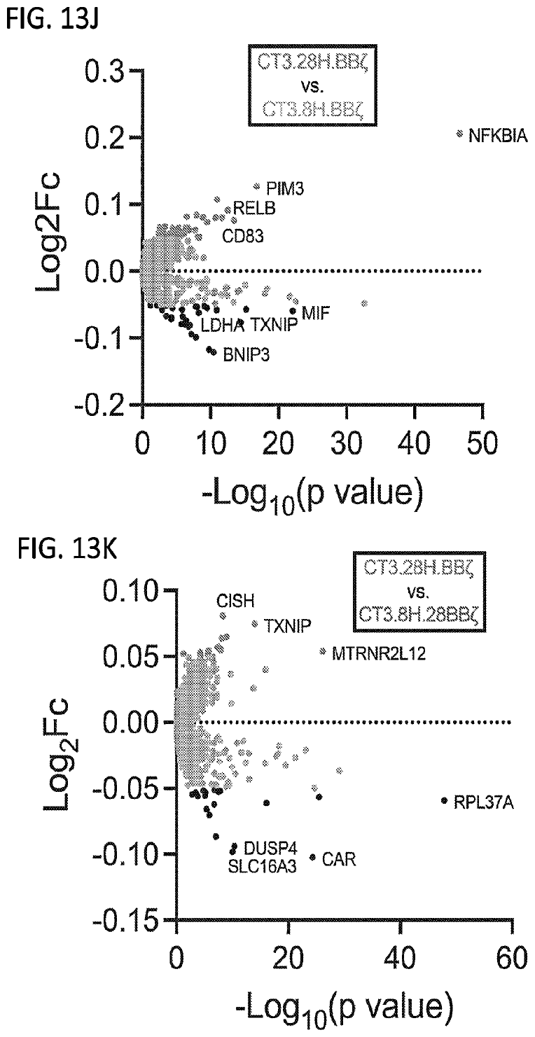

CAR T cells from two donors were manufactured and their transcriptomes were analyzed prior to injection into the mice using the droplet-based 10× Genomics platform. After quality control and filtering, a total of 14,169 single-cell transcriptomes were obtained for Donor 1 and 13,515 for Donor 2 (FIG. 13A). Each subgroup was evenly represented for Donor 1, but more CT3.8H.BBζ cells and fewer UT mock cells were captured for Donor 2. To manually annotate the cellular subsets, previously published annotation strategies were followed (Wilson et al., Cancer Discov 12:2098-2119, 2022; Patil et al., Sci Immunol 3:eaan8664, 2018). Graph-based unsupervised clustering was performed and CD8 and CD4 protein expression and conventional genes were used to define T (CD8A, CD4), NK (NKG7, GNLY), and B cells (MS4A1). T cell subsets were further defined as cytotoxic effector cells by their robust expression of PRF1 and various granzyme-encoding genes and memory cells by their expression of SELL, IL7R, CD27, and LEF1. Regulatory T cells (Tregs) were identified by IL2RA and FOXP3. Cell clusters were defined as proliferating when they expressed classic proliferation and cell cycle-related genes (e.g., TOP2A, MKI67, CCNB1/2, minichromosome maintenance (MCM) complex, or histone genes). The T cell injection product of Donor 1 was composed of 12 cell clusters (FIG. 13B), which were distinctly separated by their CD8 (22.3%) and CD4 (77.7%) protein expression (FIG. 13C). By using compound gene signatures, it was found that this donor contained predominantly proliferating CD8⁺ and CD4⁺ T cells (72.8%) and CD8⁺ cytotoxic effector cells (33.1%) at the end of the CAR T manufacturing process (FIGS. 13D-13E). A fraction of the CD8⁺ cells were exhausted cytotoxic effector cells (FIGS. 13D-13E). Donor 2 consisted of 15 independent cell clusters (FIG. 13F). Like Donor 1, the total cell population contained fewer CD8⁺ (37.9%) than CD4⁺ T cells (62.1%; FIG. 13G) of which 25.6% were cytotoxic T cells and 1.7% Tregs (FIGS. 13H-13I).

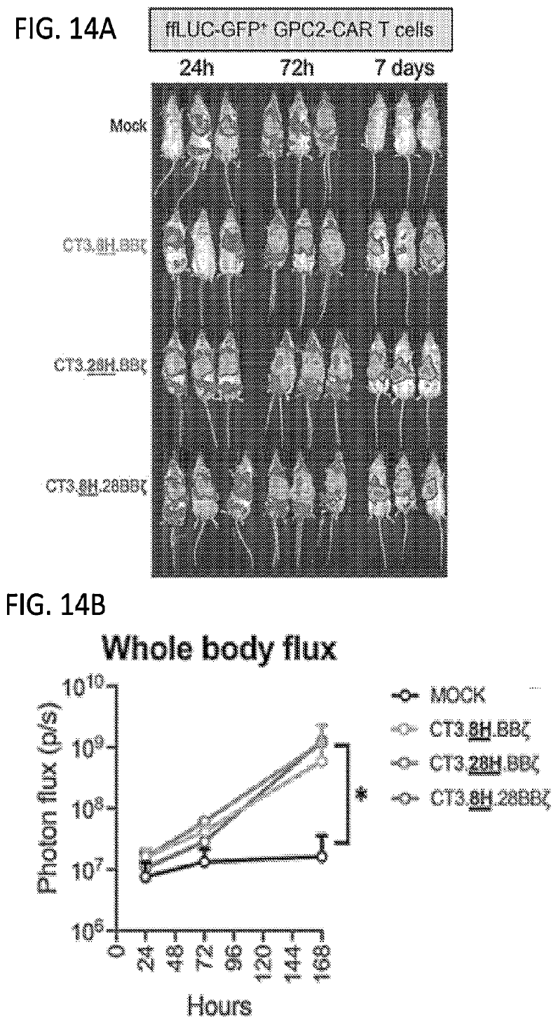

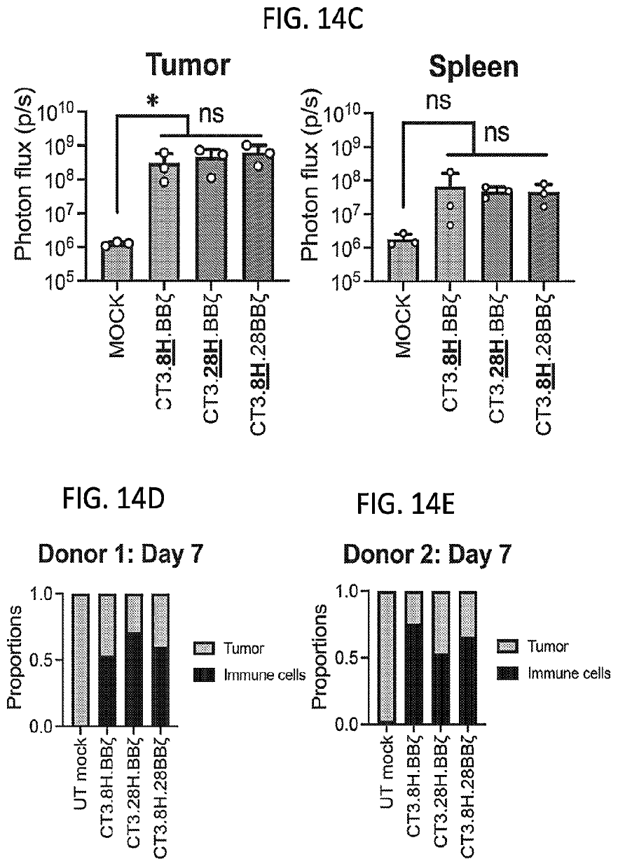

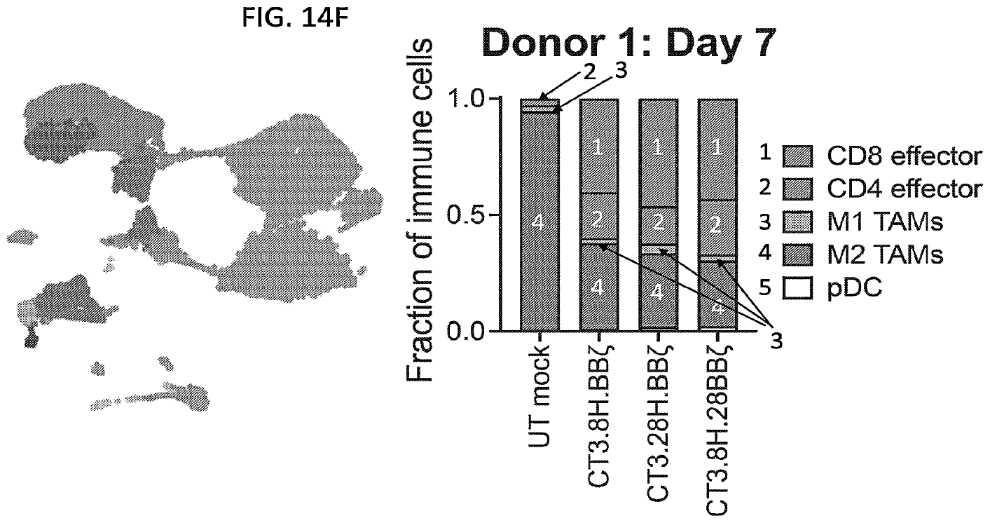

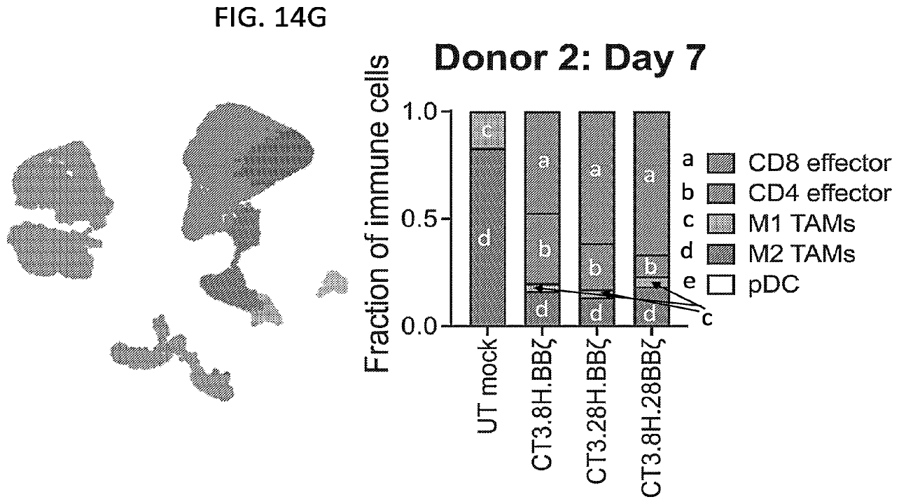

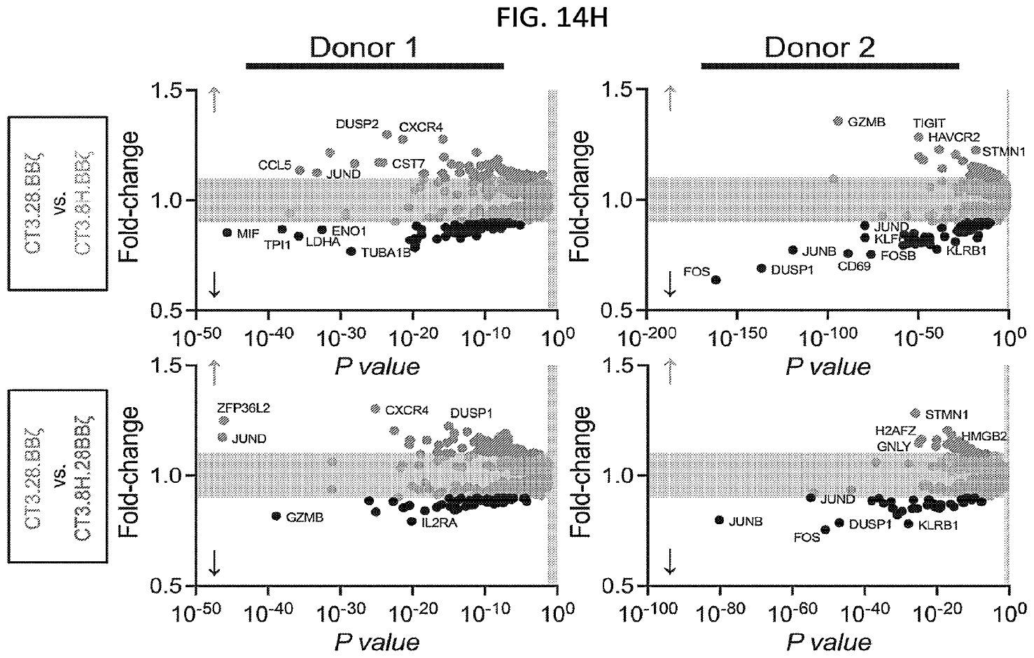

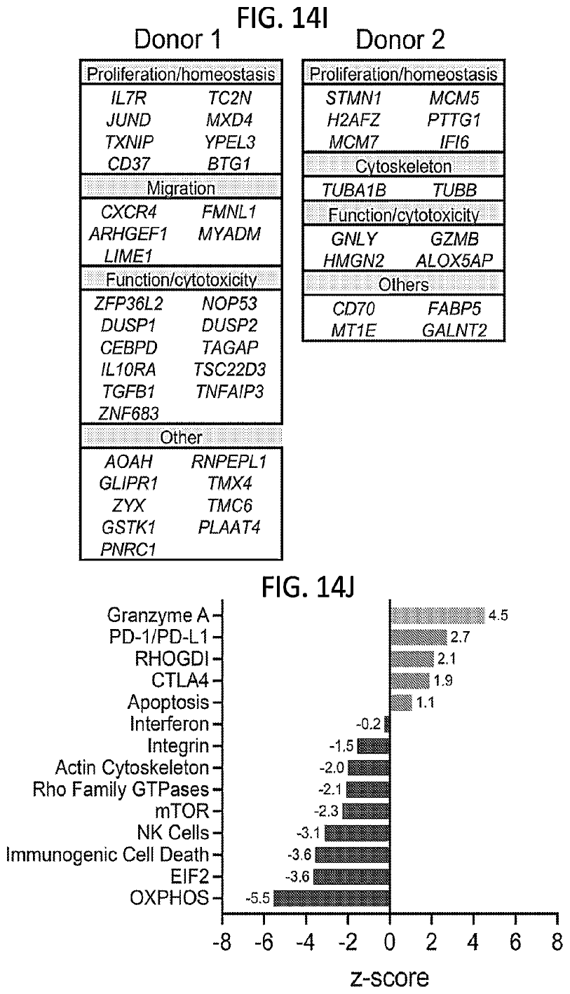

To determine the ideal time point for single-cell RNA-seq analysis of the TME after T cell injection into mice, the distribution and expansion of CAR T cells in vivo were evaluated. To track the cells, GPC2-CAR T cells were transduced to express firefly luciferase-GFP (ffLUC-GFP). Three weeks following tumor implantation into the right adrenal gland fat pad, GPC2-CAR-ffLUC-GFP T cells were injected and serial BLI was conducted. T cells in all four test groups first accumulated in the lungs and femurs and gradually expanded over the next 48 hours (FIG. 14A). By Day 7, the overall BLI signal had faded in the UT mock T group, while all three GPC2-CAR groups demonstrated an increase in the signal confined to the tumor and spleen, consistent with local T cell expansion (FIGS. 14B-14C).

After CAR T-cell injection, tumors from IMR-5-bearing mice (Day 8) were found to contain large proportions of CD8 and CD4 effector T cells comprising approximately 50% or more of the cells from the TME (FIGS. 14D-14G). In contrast, UT mock cells accounted for <3% of the cells in the TME. This is consistent with the results from in vivo tracking experiments, where UT mock T cells failed to expand and engraft (FIG. 14A). The residual cells in this group were almost exclusively M2 tumor-associated macrophages. Tumor cells were distinguished from immune cells by their gene expression and copy number variation profile.

Trajectory analysis of the immune cells in vivo revealed that the few numbers of antigen-presenting cells present at the time of injection were soon outnumbered by CD4 and CD8 T cells. These cells developed from a state of high proliferative capacity (marked by expression of MKI67) to terminally differentiated and dysfunctional CD69-expressing, EOMES-expressing, and TOX-expressing effector cells or transitioned to a memory phase evident by expression abundance of IL7RA, LEF1, and CCL5. To better characterize the transcriptome of tumor-infiltrating CD8⁺ effector cells across the GPC2-CAR T cell groups, the gene expression profile of CT3.28H.BBζ was compared with that of the two other GPC2-targeted CARs (FIG. 14H). In Donor 1, 33 differentially expressed genes (DEGs) were found that were shared by both analyses (CT3.28H.BBζ vs CT3.8H.BBζ and CT3.28H.BBζ vs CT3.8H.28BBζ; FIG. 14I). In Donor 2, there were 16 shared DEGs (FIG. 14I). Compared with the other two CAR T cell groups, CT3.28H.BBζ CAR T cells exhibited upregulation of CXCR4, ARHGEF1, and LIME1, which are implied in chemokine-related T cell migration and T cell renewal (Bouafia et al., J Clin Invest 129:1047-1060, 2019; Chaix et al., J Immunol 193:1013-1016, 2014; Park et al., Mol Cells 43:921-934, 2020). Other DEGs include IL7R, JUND, ZFP36L, and TXNIP, which are important in T-cell homeostasis and memory formation (Schlins et al., Nat Inmnunol 1:426-432, 2000; Meixner et al., Embo J 23:1325-1335, 2004; Ruppert et al., PLoS One 7:e32262, 2012; Muri et al., Eur J Immunol 51:115-124, 2021; Petkau et al., Nat Commun 13(1):2274, 2022). CT3.28H.BBζ CAR T cells also had upregulated genes encoding effector molecules (e.g., GNLY, GZMB, ZNF683, and HMGN2) and cell cycle components (e.g., STMN1, MCM5, MCM7, and PTTG1). Lastly, pathway analysis corroborated these findings, demonstrating activation of the granzyme A pathway (z-score: 4.54; p value=1.16E-35). CT3.28H.BBζ CAR T cells also showed a downregulation of the EIF2 (z-score: −3.628; p value=6.19E-54) and oxidative phosphorylation pathways (z-score: −5.53; p value=1.85E-56; FIG. 14J). Pairwise DEG analysis revealed that compared with the other two CARs, CT3.28H.BBζ already expressed genes important in the regulation of pathways of T cell exhaustion (e.g., NFKBIA, CISH), genes that promote T cell activation and proliferation (e.g., CD83, TXNIP, LDHA), and genes that may prevent apoptosis (e.g., MTRNR2L12; FIGS. 14J-14K). These findings indicate that at the time of manufacturing these cells, there are already transcriptomic differences that may influence the divergent cytotoxic activity and survival of the cells in vivo.

Taken together, these findings demonstrate that all GPC2-targeted CAR T cells substantially expand towards a cytotoxic effector population in vivo. Furthermore, CT3.28H.BBζ CAR T cells upregulate effector molecules and genes involved in T cell migration and memory homeostasis. These findings may be responsible for the superior antitumor cytotoxicity observed in mice treated with CT3.28H.BBζ CAR T cells compared with the other CAR therapy groups.

### Example 11: CT3.28H.BBζ Outperforms K666.28H.BBζ with Superior Antitumor Activity Against GD2⁺ GPC2^{low} NB

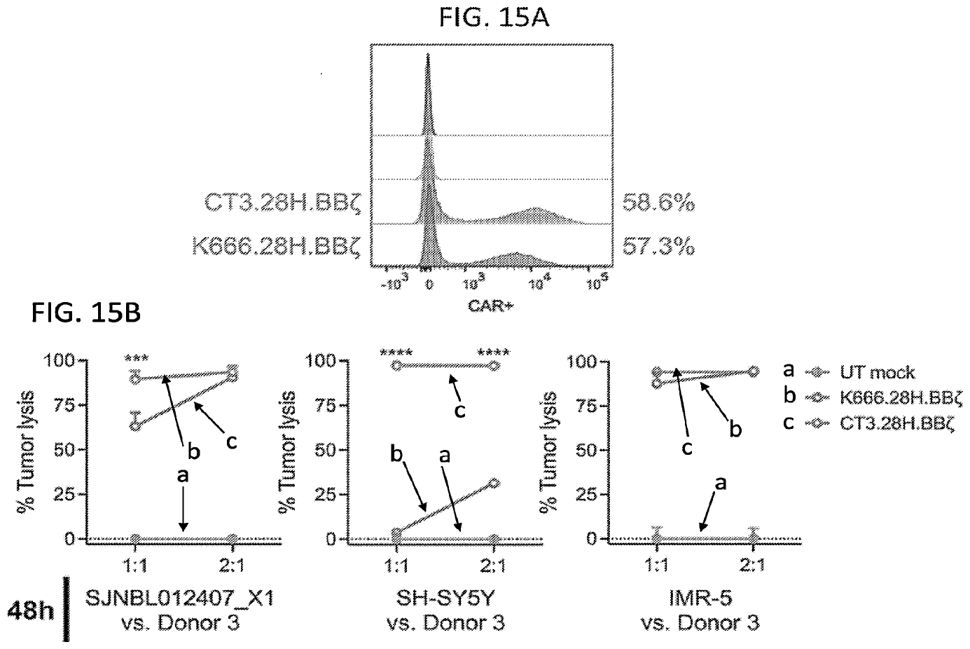

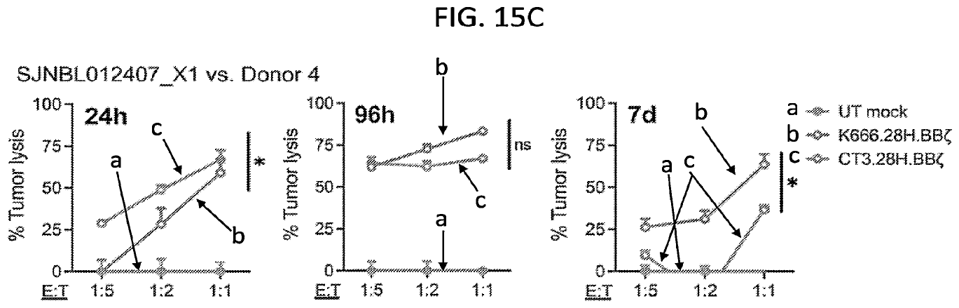

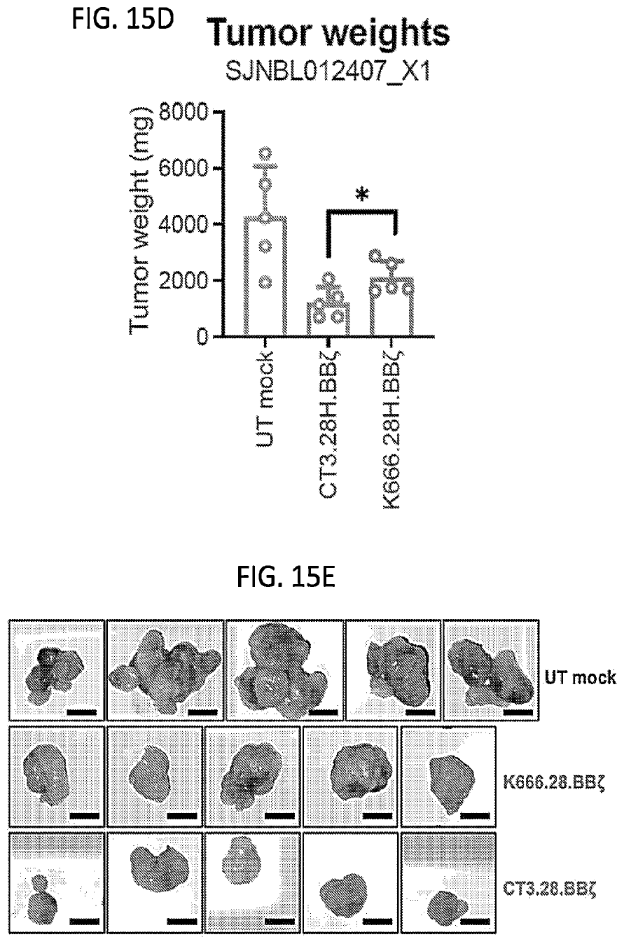

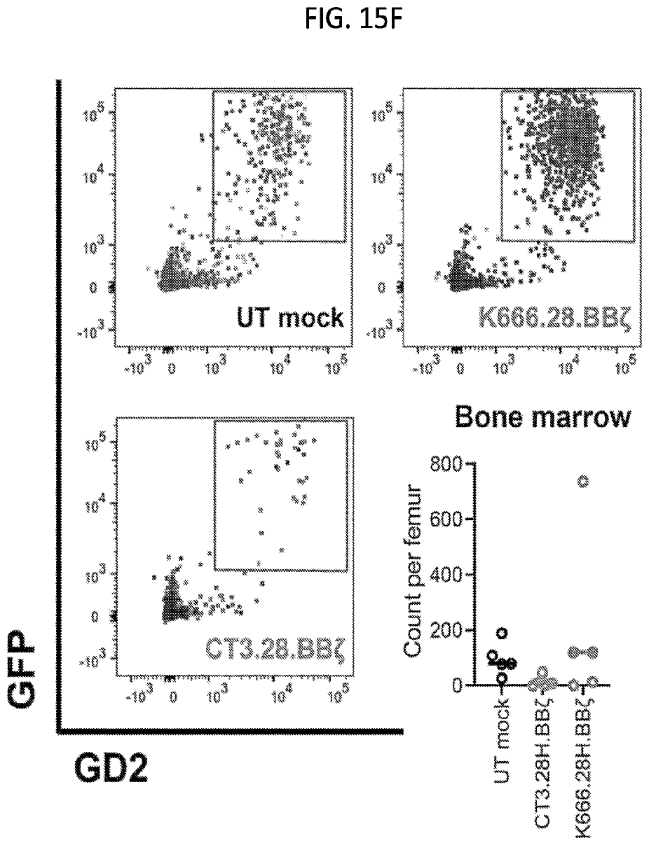

Previous CAR T cell trials in NB were conducted with K666-based and 14.18-scFv-based GD2-CAR T cells (Straathof et al., Sci Transl Med 12:eabd6169, 2020; Heczey et al., Mol Ther 25:2214-2224, 2017; Louis et al., Blood 118:6050-6056, 2011; Pule et al., Nat Med 14:1264-1270, 2008). Although the trials reported tolerability, very few of the treated patients achieved objective responses. Next, studies were performed to assess how CT3.28H.BBζ compared in function to existing CAR T-cell therapies targeting GD2. The aim was to compare the preclinical activity of the K666-based and 14.G2a-based GD2-targeting CARs (Straathof et al., Sci Transl Med 12:eabd6169, 2020) with that of CT3.28H.BBζ. To create comparable testing conditions, the scFvs were cloned into the identical CAR construct used for CT3.28H.BBζ and CT3.8H.BBζ and performed head-to-head comparisons with serial tumor rechallenges in vitro. Subsequently, K666.28H.BBζ was chosen and the anti-NB activity of CT3.28H.BBζ was further compared with that of K666.28H.BBζ in vitro and in vivo. The CAR T cells demonstrated comparable transduction efficiencies (FIG. 15A). ffLUC-GFP-expressing SJNBL012407_X1, IMR-5 (both MYCN-amplified), and SH-SY5Y (MYCN-WT) were incubated with CT3.28H.BBζ or K666.28H.BBζ CAR T cells at varying E:T ratios. These NB cells have different expression levels of GPC2 and GD2. After 48 hours, tumor cell lysis was determined by applying a luciferase reporter assay (FIG. 15B). At an E:T ratio of 1:1, CT3.28H.BBζ CAR T cells showed superior anti-NB cytotoxicity compared with K666.28H.BBζ CAR T cells against the GD2^{intermediate} GPC2^{low} PDX and GD2^{low} GPC2^{low} SH-SY5Y. The tumor lysis was comparable in GD2^{high} GPC2^{high} IMR-5 in both groups. Next, testing was expanded to an in vitro tumor rechallenge model with SJNBL012407_X1. The cytotoxic capacity of the two CARs was measured at 24 hours, Day 4, and Day 7 following daily tumor rechallenges. Although CT3.28H.BBζ CAR T cells showed better anti-NB cytotoxicity at first, their activity gradually decreased over time, rendering K666.28H.BBζ CAR T cells superior on Day 7 (FIG. 15C). Next, the antitumor activity of the two CARs was compared in vivo. SJNBL012407_X1-bearing mice were injected with 5×10⁶ CAR⁺ T cells on Day 21 after tumor inoculation. On Day 50 post tumor injection, the primary tumors were weighed, and the bone marrow was analyzed for residual NB cells. Tumor weights were smaller after therapy with CT3.28H.BBζ CAR T cells compared with K666.28H.BBζ CAR T (FIGS. 15D-15E). Furthermore, 3 out of 5 mice with K666.28H.BBζ CAR T cells had higher levels of detectable tumor cells in their bone marrow than all mice treated with CT3.28H.BBζ CAR T cells (FIG. 15F). A possible cause for therapy resistance after K666.28H.BBζ CAR T therapy could be the downregulation of GD2 in the primary tumors. These findings demonstrate that CT3.28H.BBζ CAR T cells outperformed K666.28H.BBζ CAR T within an early time window in vitro as well as in an orthotopic NB-PDX in vivo model, leading to better tumor control of the primary tumor and a trend toward better control of metastatic disease burden in the bone marrow.

It will be apparent that the precise details of the methods or compositions described may be varied or modified without departing from the spirit of the described aspects of the disclosure. We claim all such modifications and variations that fall within the scope and spirit of the claims below.

1. A chimeric antigen receptor (CAR), comprising:
an extracellular antigen-binding domain that specifically binds glypican-2 (GPC2), comprising a variable heavy (VH) domain and a variable light (VL) domain, wherein the VH domain comprises the complementarity determining region 1 (CDR1), CDR2 and CDR3 sequences of SEQ ID NO: 2 and the VL domain comprises the CDR1, CDR2 and CDR3 sequences of SEQ ID NO: 4;
a CD28 hinge region;
a CD28 transmembrane domain;
an intracellular co-stimulatory domain; and
an intracellular signaling domain.

2. The CAR of claim 1, wherein the CDR1, CDR2 and CDR3 sequences are defined using the Kabat, IMGT or Paratome numbering schemes, or a combination of the Kabat, IMGT and Paratome numbering schemes.

3. The CAR of claim 1, wherein:
the VH domain CDR1, CDR2 and CDR3 sequences respectively comprise residues 31-35, 50-66 and 99-112 of SEQ ID NO: 2 and the VL domain CDR1, CDR2 and CDR3 sequences respectively comprise residues 24-33, 49-55 and 88-96 of SEQ ID NO: 4;
the VH domain CDR1, CDR2 and CDR3 sequences respectively comprise residues 26-33, 51-58 and 97-112 of SEQ ID NO: 2 and the VL domain CDR1, CDR2 and CDR3 sequences respectively comprise residues 27-31, 49-51 and 88-96 of SEQ ID NO: 4;
the VH domain CDR1, CDR2 and CDR3 sequences respectively comprise residues 26-35, 47-61 and 97-112 of SEQ ID NO: 2 and the VL domain CDR1, CDR2 and CDR3 sequences respectively comprise residues 27-33, 45-55 and 88-95 of SEQ ID NO: 4; or
the VH domain CDR1, CDR2 and CDR3 sequences respectively comprise residues 26-35, 47-66 and 97-112 of SEQ ID NO: 2 and the VL domain CDR1, CDR2 and CDR3 sequences respectively comprise residues 24-33, 45-55 and 88-96 of SEQ ID NO: 4.

4. The CAR of claim 1, wherein:
the amino acid sequence of the VH domain is at least 90% identical to SEQ ID NO: 2, and comprises the CDR1, CDR2 and CDR3 sequences of SEQ ID NO: 2; and
the amino acid sequence of the VL domain is at least 90% identical to SEQ ID NO: 4, and comprises the CDR1, CDR2 and CDR3 sequences of SEQ ID NO: 4.

5. The CAR of claim 1, wherein the VH domain and the VL domain sequences are humanized.

6. The CAR of claim 5, wherein:
the amino acid sequence of the VH domain comprises residues 1-123 of SEQ ID NO: 8, and the amino acid sequence of the VL domain comprises residues 139-244 of SEQ ID NO: 8;
the amino acid sequence of the VH domain comprises residues 1-122 of SEQ ID NO: 12, the amino acid sequence of the VL domain comprises residues 138-243 of SEQ ID NO: 12;
the amino acid sequence of the VH domain comprises residues 1-122 of SEQ ID NO: 16, and the amino acid sequence of the VL domain comprises residues 138-244 of SEQ ID NO: 16; or
the amino acid sequence of the VH domain comprises residues 1-122 of SEQ ID NO: 20, and the amino acid sequence of the VL domain comprises residues 138-243 of SEQ ID NO: 20.

7. The CAR of claim 1, wherein the extracellular antigen-binding domain comprises or consists of the amino acid sequence of SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20 or SEQ ID NO: 22.

8. The CAR of claim 1, wherein
the CD28 hinge region comprises or consists of the amino acid sequence of SEQ ID NO: 24, or
the CD28 transmembrane domain comprises or consists of the amino acid sequence of SEQ ID NO:26.

9. (canceled)

10. The CAR of claim 1, wherein the co-stimulatory domain comprises a 4-1BB signaling moiety.

11. (canceled)

12. The CAR of claim 1, wherein the signaling domain comprises a CD3 ζ signaling domain.

13. (canceled)

14. The CAR of claim 1, wherein the amino acid sequence of the CAR comprises or consists of SEQ ID NO: 38.

15. An isolated cell expressing the CAR of claim 1.

16. The isolated cell of claim 15, wherein the cell is an immune cell or an induced pluripotent stem cell (iPSC).

17. The isolated cell of claim 16, wherein the immune cell is a T cell, a B cell, a natural killer (NK) cell or a macrophage.

18. An isolated nucleic acid molecule encoding the CAR of claim 1.

19. The isolated nucleic acid molecule of claim 18, comprising or consisting of SEQ ID NO: 37 or comprising or consisting of nucleotides 73-1470 of SEQ ID NO: 37.

20. (canceled)

21. The isolated nucleic acid molecule of claim 18 operably linked to a promoter.

22. The isolated nucleic acid molecule of claim 21, wherein the promoter is the human elongation factor 1α (EF1α) promoter.

23. A vector comprising the isolated nucleic acid molecule claim 18.

24. (canceled)

25. An isolated cell comprising the isolated nucleic acid molecule of claim 18.

26. (canceled)

27. (canceled)

28. A composition comprising a pharmaceutically acceptable carrier and the CAR of claim 1, an isolated cell expressing the CAR of claim 1, an isolated nucleic acid molecule encoding the CAR of claim 1, or a vector comprising an isolated nucleic acid molecule encoding the CAR of claim 1.

29. A method of treating a GPC2-positive cancer in a subject, inhibiting tumor growth of a GPC2-positive cancer in a subject, or inhibiting metastasis of a GPC2-positive cancer in a subject, comprising administering to the subject a therapeutically effective amount of the CAR of claim 1, an isolated cell expressing the CAR of claim 1, an isolated nucleic acid molecule encoding the CAR of claim 1, or a vector comprising an isolated nucleic acid encoding the CAR of claim 1.

30. (canceled)

31. The method of claim 29, wherein:
the GPC2-positive cancer is a solid tumor,
the GPC2-positive cancer is a pediatric cancer, and/or
the GPC2-positive cancer is a neuroblastoma, medulloblastoma, retinoblastoma, acute lymphoblastic leukemia, embryonal rhabdomyosarcoma, alveolar rhabdomyosarcoma, Ewing's sarcoma, desmoplastic small round cell tumor, glioma or osteosarcoma.

32. (canceled)

33. (canceled)

34. The method of claim 29, further comprising administering chemotherapy to the subject.
